(19) United States
(12) Patent Application Publication
(10) Pub. No.: US 2005/0175696 A1
(43) Pub. Date: Aug. 11, 2005

(54) Drug granule coatings that impart smear resistance during mechanical compression

(76) Inventors: David Edgren, Los Altos, CA (US); Alya Khan, San Mateo, CA (US); Abdul Majid Qureshi, Menlo Park, CA (US); James Ergun, Burlingame, CA (US); Satishkumar Ramachandran, Belmont, CA (US); Noymi Yam, Sunnyvale, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/024,329

(22) Filed: Dec. 28, 2004

Related U.S. Application Data
(60) Provisional application No. 60/533,122, filed on Dec. 30, 2003.
(60) Provisional application No. 60/533,470, filed on Dec. 29, 2003.

(51) Int. Cl. A61K 9/48; A61K 9/26; A61K 31/7008

(52) U.S. Cl. 424/470; 514/23

(57) ABSTRACT

A drug formulation is disclosed comprising granules having a substrate and a coating, said granule substrate comprising a solubilizing surfactant or a low solubility therapeutic drug, or both, and said granule coating comprising a hydrophilic polymer. Also disclosed is a drug formulation consisting of a tablet core made by mechanical compression, wherein said tablet core comprises granules having a substrate and a coating, said granule substrate comprising a solubilizing surfactant or a low solubility therapeutic drug, or both, and said granule coating comprising a hydrophilic polymer. Also disclosed is a dosage form for oral administration of topiramate, comprising a tablet core and an osmotic delivery system. Methods for controlling topiramate release patterns by altering the composition of the topiramate dosage form are also disclosed.

### CROSS REFERENCE TO RELATED APPLICATION

This application claims the benefit of U.S. Provisional Application No. 60/533,112, filed on Dec. 29, 2003 and U.S. Provisional Application No. 60/533,470, filed on Dec. 29, 2003, which are incorporated by reference herein in thier entirety.

### FIELD OF THE INVENTION

A drug formulation is disclosed comprising a compressed tablet core comprising a solubilizing agent, preferably a surfactant, and a low solubility therapeutic drug, in which one or both of the therapeutic drug and solubilizing agent are in coated granules. Also disclosed is a controlled release dosage form for oral administration of topiramate, comprising the tablet core. A method is disclosed for controlling topiramate release patterns by modifying the composition of the coated granules in the controlled release dosage form.

### BACKGROUND OF THE INVENTION

Various dosage forms for the controlled release of pharmaceutical agents are known. While a variety of dosage forms for delivering certain drugs may be known, not every drug may be suitably delivered from those dosage forms because of solubility, metabolic processes, absorption and other physical, chemical and physiological parameters that may be unique to the drug and the mode of delivery.

Dosage forms using surfactants are known in the art. U.S. Pat. No. 6,569,463 describes using drug formulations consisting of coated granules, in which the coating consists of at least one surfactant and preferably a mixture of the surfactant with a hydrophobic drug and a lipophilic additive. This substrate coating facilitates rapid dispersion and provides rapid, sustained solubilization of the drug in the absence of liquid ingredients. The lipophilic additive further enhances solubilization of the drug or promotes dispersion in vivo.

Dosage forms that incorporate a low solubility drug, including high drug loading for the dosage form, continue to be an elusive goal for controlled release delivery technology, in which dosage forms generally tend to be too large for patients to willingly swallow them.

Dosage forms made in the form of an osmotic delivery system are known to the delivery art in U.S. Pat. Nos. 3,845,770 and 3,916,899, both issued to patentees Felix Theeuwes and Takeru Higuchi. The dosage form disclosed and claimed in these patents comprises a semipermeable wall that surrounds a compartment containing a beneficial agent, usually a beneficial drug (e.g. a pharmaceutical or therapeutic drug).

The wall is permeable to the passage of an external fluid and substantially impermeable to the passage of a beneficial drug. There is at least one passageway through the wall for delivering the beneficial agent from the dosage form. The dosage form releases the beneficial agent by fluid being continuously imbibed through the wall into the dosage form at a rate determined by the permeability of the semipermeable wall and the osmotic pressure gradient across the wall. This physical-chemical action produces a solution containing the beneficial agent that is hydrodynamically dispensed through the passageway from the dosage form.

The above described patents also are useful for delivering a beneficial agent that exhibits a low solubility drug in an external fluid imbibed into the dosage form. The dosage form delivers such beneficial agent by blending the drug with an osmotically—effective solute, known also as osmagent. The osmagent in the dosage form is a substantial motive force as it exhibits an osmotic pressure gradient across the wall of the dosage form, and it imbibes fluid into the dosage form. The osmagent produces a solution with the imbibed fluid that is osmotically delivered from the dosage form concomitantly transporting therewith undissolved, or dissolved drug from the dosage form.

The dosage form of these patents significantly advanced the delivery art, and its delivery kinetics are ideal for many drugs. However, when a low solubility drug is mixed with an osmagent to produce an equilibrium ratio, the resulting drug solubility in the presence of the osmagent often is still too low, usually less than 100 mg/ml. In this instance, this drug cannot be released at a controlled rate over a prolonged period of time. When the resulting beneficial agent's solubility is low, it is difficult to deliver the beneficial agent at meaningful therapeutic rates.

Another improvement is described in U.S. Pat. No. 4,755,180. This patent teaches encapsulated osmotic agents as a means to improve the delivery kinetics of the dosage form. The shortcomings of this approach, however, are all too apparent in that it is still based on controlling the delivery rate solely by means of an osmotic gradient across the outer wall of the dosage form.

Various devices in which a drug composition is delivered as a slurry, suspension or solution from a small exit orifice by the action of an expandable layer are described in U.S. Pat. Nos. 5,633,011; 5,190,765; 5,252,338; 5,620,705; 4,931,285; 5,006,346; 5,024,842; and 5,160,743. Typical devices include a tablet comprising an expandable push layer and a drug layer, which tablet is surrounded by a semipermeable membrane having a delivery orifice. In certain instances, the tablet is provided with a subcoat to delay release of the drug composition to the environment of use.

Devices in which a drug composition is delivered in a dry state from a large exit orifice by the action of an expandable layer are described in U.S. Pat. Nos. 4,892,778, 4,915,949 and 4,940,465 and 5,023,088. These references describe a dispenser for delivering a beneficial agent to an environment of use that includes a semipermeable wall containing a layer of expandable material that pushes a dry drug layer composition out of the compartment formed by the wall. The exit orifice in the device is substantially the same diameter as the inner diameter of the compartment formed by the wall. In such devices, a substantial area of the drug layer composition is exposed to the environment of use leading to release performance that can be subject to the stirring conditions in such environment.

Other similar devices have delivered drug by expelling discrete drug containing tablets at a controlled rate over time. U.S. Pat. Nos. 5,938,654; 4,957,494; 5,023,088; 5,110,597; 5,340,590; 4,824,675; and 5,391,381.

Other devices attempt to deliver low solubility drugs by incorporating liquid drug formulations that are released at a controlled rate over time. These devices are disclosed in U.S. Pat. Nos. 4,111,201; 5,324,280; 5,413,672; 6,174,547. However, such liquid osmotic delivery systems are limited in the concentration of drug in the liquid formulation and hence, the drug loading available, leading to delivery systems that can be of an unacceptably large size.

Still other delivery systems utilize a liquid carrier to deliver tiny time pills suspended within the liquid carrier. Such devices are disclosed U.S. Pat. Nos. 4,853,229 and 4,961,932. These suspensions require that the therapeutic dose of pharmaceutical agent be dispensed by volume with measuring devices such as graduated cylinders or measuring spoons, a dispensing process that can be messy and inconvenient for the patient to administer.

While dosage forms delivering the drug composition to the environment of use in the dry state through a large delivery orifice may provide suitable release of drug over a prolonged period of time, the exposure of the drug layer to the variably turbulent fluid environment of use such as the upper gastrointestinal tract may result in agitation-dependent release of drug that in some circumstances is difficult to control. Moreover, such dosage forms delivering in the dry state into a semisolid environment lacking sufficient volumes of bulk water such as in the lower colonic environment of the gastrointestinal tract may have difficulty liberating the dry dispensed drug composition into the environment as the high solids content composition tends to adhere to the dosage form at the site of the large orifice. Accordingly, it may be advantageous to release the drug as a well-hydrated slurry or suspension that may provide a means to control the rate of expansion of the push layer and in combination with the smaller size of the exit orifice in the dosage form to minimize effects of localized stirring conditions on delivery performance. Move this paragraph to just before [00012]??

The dosage forms described above deliver therapeutic agents at a substantially zero order rate of release. Recently, dosage forms have been disclosed for delivering certain drugs at substantially ascending rates of release such as ALZA Corporation's Concerta® methylphenidate product. PCT Published Application Nos. U.S. 99/11920 (WO 9/62496); U.S. 97/13816 (WO 98/06380); and U.S. 97/16599 (WO 98/14168). Such disclosed dosage forms involve the use of multiple drug layers with sequentially increasing concentrations of drug in each drug layer to produce the increasing delivery rate of drug over time. While such multi-layer tablet constructions represent a significant advancement to the art, these devices also have limited capability of delivering lowly soluble pharmaceutical agents, particularly those associated with relatively large doses of such agents, in a size that is acceptable for patients to swallow.

Thus, there remains a need for a means to manufacture and deliver high doses of lowly soluble drug compounds at various delivery patterns that are convenient and feasible for patients in need to swallow. The need includes effective dosing methods, dosage forms and devices that will permit the controlled release of the drug compounds over a prolonged period of time by increasing the solubility of the active agent in order to increase the time between dosing, preferably twice a day and most preferably to obtain a once-a-day dosing regimen. Such dosage forms should preferably have the option of delivering at a substantially zero order rate of release, ascending or other hybrid delivery rate pattern appropriate for the therapeutic agent being delivered.

### SUMMARY OF THE INVENTION

The present invention is directed to a drug composition comprising granules having a substrate and a coating, the granule substrate comprising a solubilizing agent, preferably a surfactant, or a low solubility therapeutic drug, or both, and the granule coating comprising a hydrophilic polymer.

One present invention is a drug formulation comprising a tablet core made by mechanical compression, in which the tablet core comprises granules having a substrate and a coating, the granule substrate comprising a solubilizing agent, preferably a surfactant, or a low solubility therapeutic drug, or both, and the granule coating comprising a hydrophilic polymer.

Another present invention is a drug formulation comprising a capsule, wherein said capsule comprises granules having a substrate and a coating, the granule substrate comprising a solubilizing agent, preferably a surfactant, or a low solubility therapeutic drug, or both, and the granule coating comprising a hydrophilic polymer.

An embodiment of the invention is a drug formulation, in which the granule substrate comprises a surfactant. Another embodiment is a drug formulation, in which the granule substrate further comprises the drug. In yet another embodiment is a drug formulation, in which the granule substrate comprises the drug. A related embodiment is a drug formulation, in which the amount of solubilizing agent, preferably surfactant, in the granule substrate is between about 1% and about 90% by weight. A preferred embodiment is a drug formulation in which the amount of solubilizing agent, preferably surfactant, in the granule substrate is between about 5% and about 50% by weight.

Another aspect of the invention is a drug formulation, in which the solubilizing agent, preferably surfactant, is selected from the group consisting of polyoxyl 40 stearate, polyoxyl 50 stearate, triblock co-polymers of ethylene oxide/propylene oxide/ethylene oxide, sorbitan monopalmitate, sorbitan monostearate, glycerol monostearate, polyoxyethlene stearate, polyoxyethylene 40 sorbitol lanolin derivative, polyoxyethylene 75 sorbitol lanolin derivative, polyoxyethylene 6 sorbitol beeswax derivative, polyoxyethylene 20 sorbitol beeswax derivative, polyoxyethylene 20 sorbitol lanolin derivative, polyoxyethylene 50 sorbitol lanolin derivative, polyoxyethylene 23 lauryl ether, polyoxyethylene 23 lauryl ether, polyoxyethylene 2 cetyl ether, polyoxyethylene 10 cetyl ether, polyoxyethylene 20 cetyl ether, polyoxyethylene 2 stearyl ether, polyoxyethylene 10 stearyl ether, polyoxyethylene 20 stearyl ether, polyoxyethylene 21 stearyl ether, polyoxyethylene 20 oleyl ether, polyoxyethylene 40 stearate, polyoxyethylene 50 stearate, polyoxyethylene 100 stearate, sorbitan monopalmitate, sorbitan monostearate, sorbitan tristearate, polyoxyethylene 4 sorbitan monostearate, polyoxyethylene 20 sorbitan tristearate, and mixtures thereof. A preferred embodiment is a drug formulation, in which the surfactant is selected from the group consisting of polyoxyl 40 stearate, polyoxyl 50 stearate, di-block copolymers of ethylene oxide:propylene oxide, a:b:a triblock copolymers of ethylene oxide:propylene oxide:ethylene oxide and mixtures thereof.

Another aspect of the invention is a drug formulation, in which the granule substrate further comprises a viscosity agent. A further embodiment of the invention is a drug formulation, in which the granule substrate further comprises a binding agent. Another further embodiment is a drug formulation, in which the granule substrate further comprises an osmotic agent, and more preferably, an osmotic agent selected from the group consisting of sodium chloride, sucrose, sorbitol, and mannitol.

Another aspect of the invention is a drug formulation, in which the therapeutic agent in the absence of solubilizing agent, preferably, surfactant has aqueous solubility that is between about 1 μg/ml and about 100 mg/ml. A preferable embodiment is a drug formulation, in which the therapeutic agent in the absence of solubilizing agent, preferably, surfactant, has aqueous solubility that is between about 1 μg/ml and about 50 mg/ml.

Another embodiment of the invention is a drug formulation, in which the granule coating is continuous. An alternative embodiment is a drug formulation, in which the granule coating is discontinuous.

Another embodiment of the invention is a drug formulation, in which the granule coating is water-soluble, and more preferably, in which the granule coating is comprised of a compound selected from the group consisting of polyvinyl pyrrolidone, polyethylene oxide, polyethylene glycol, polyvinyl alcohol, polyvinyl alcohol-polyethylene glycol copolymer, vinyl acetate-vinyl pyrrolidone copolymer, hydroxypropyl cellulose, hydroxypropyl methylcellulose, methylcellulose, hydroxyethyl cellulose, sodium carboxymethylcellulose, calcium carboxymethylcellulose, polyvinyl acetate polyethylene glycol co-polymer, starches, maltodextrins, sugars, sorbitol, mannitol, sucrose, gelatin, casein, natural gums, alginates and mixtures thereof.

A related embodiment of the invention is a drug formulation, in which the granule coating is water insoluble, and more preferably, in which the granule coating is comprised of a compound selected from the group consisting of a polyether, a polyester, cellulose acetate, cellulose acetate butyrate, a polyacrylate and mixtures thereof. Another embodiment is a drug formulation, in which the granule coating is additionally comprised of a compound selected from the group consisting of polyethylene glycol, polyoxyethylene-polyoxypropylene copolymer, hydroxypropyl cellulose, glycerin, a citric acid ester, and dibutyl sebacate. In a preferred embodiment is a drug formulation, in which the polyether of the granule coating is ethyl cellulose. In another preferred embodiment is a granule coating, in which the polyester is polyvinyl acetate. In yet another preferred embodiment is a granule coating, in which the polyacrylate is an ethylacrylate-methylmethacrylate copolymer. In yet another preferred embodiment is a granule coating, in which the citric acid ester is triethyl citrate or acetyl triethylcitrate,

Another embodiment of the invention is a drug formulation, in which the granule coating is a single layer. A preferable embodiment is a drug formulation, in which the single-layer coating is comprised of polyvinyl pyrrolidone or polyethylene oxide. In another embodiment is a drug formulation, in which the single-layer coating is between about 1% and about 3% of the granule weight, or at least about about 24% of the granule weight.

Another embodiment is a drug formulation, in which the granule coating is a multiplicity of layers. A preferred embodiment is a drug formulation, in which the coating comprises two layers. A more preferred embodiment is a drug formulation, in which the coating is comprised of an outer layer of methylcellulose and an inner layer of polyethylene oxide or polyvinyl pyrrolidone.

In an embodiment is a dosage form for oral administration of topiramate, comprising any of the foregoing drug formulations, in which the drug is topiramate.

Another present invention is a dosage form for oral administration of topiramate, comprising a tablet core made by mechanical compression, in which the tablet core comprises granules having a substrate and a coating, said granule substrate comprising a solubilizing agent, preferably surfactant, or topiramate, or both, and said granule coating comprising a hydrophilic polymer.

An embodiment of the invention is a topiramate dosage form, further comprising an osmotic delivery system and a tablet core, wherein the osmotic delivery system and the tablet core are surrounded by a semipermeable membrane having a delivery port through the membrane.

Another embodiment of the invention is a topiramate dosage form, in which the tablet core is incorporated into a matrix delivery system. The matrix system may be either an erodible matrix delivery system or a non-erodible matrix delivery system.

Another embodiment of the invention is a topiramate dosage form, in which the dosage form is a controlled release topiramate dosage form.

Another embodiment of the invention is a controlled release topiramate dosage form, in which the solubilizing agent, preferably surfactant, is selected from the group consisting of polyoxyl 40 stearate, polyoxyl 50 stearate, triblock co-polymers of ethylene oxide/propylene oxide/ethylene oxide, sorbitan monopalmitate, sorbitan monostearate, glycerol monostearate, polyoxyethlene stearate, polyoxyethylene 40 sorbitol lanolin derivative, polyoxyethylene 75 sorbitol lanolin derivative, polyoxyethylene 6 sorbitol beeswax derivative, polyoxyethylene 20 sorbitol beeswax derivative, polyoxyethylene 20 sorbitol lanolin derivative, polyoxyethylene 50 sorbitol lanolin derivative, polyoxyethylene 23 lauryl ether, polyoxyethylene 23 lauryl ether, polyoxyethylene 2 cetyl ether, polyoxyethylene 10 cetyl ether, polyoxyethylene 20 cetyl ether, polyoxyethylene 2 stearyl ether, polyoxyethylene 10 stearyl ether, polyoxyethylene 20 stearyl ether, polyoxyethylene 21 stearyl ether, polyoxyethylene 20 oleyl ether, polyoxyethylene 40 stearate, polyoxyethylene 50 stearate, polyoxyethylene 100 stearate, sorbitan monopalmitate, sorbitan monostearate, sorbitan tristearate, polyoxyethylene 4 sorbitan monostearate, polyoxyethylene 20 sorbitan tristearate, and mixtures thereof. A preferred embodiment is a topiramate dosage form, in which the solubilizing agent, preferably surfactant, is selected from the group consisting of polyoxyl 40 stearate, polyoxyl 50 stearate, di-block copolymers of ethylene oxide:propylene oxide, a:b:a triblock copolymers of ethylene oxide:propylene oxide:ethylene oxide, and mixtures thereof. Most preferably, is a topiramate dosage form, in which the solubilizing agent, preferably surfactant, is a polyoxyethylene-polyoxypropylene copolymer. Another embodiment is a topiramate dosage form, in which the weight ratio of the polyoxyethylene-polyoxypropylene copolymer to topiramate is up to about 3.0.

Another embodiment of the invention is a controlled release topiramate dosage form, in which the amount of solubilizing agent, preferably surfactant, is between about 5% and about 90% by weight of the core, and preferably, between about 5% and about 50% by weight of the core.

Another embodiment is a controlled release topiramate dosage form, in which the granule coating is continuous, or alternatively, in which the granule coating is discontinuous.

Another embodiment is a controlled release topiramate dosage form, in which the granule coating is water soluble and preferably, is comprised of a compound selected from the group consisting of polyvinyl pyrrolidone, polyethylene oxide, polyethylene glycol, polyvinyl alcohol, polyvinyl alcohol-polyethylene glycol copolymer, vinyl acetate-vinyl pyrrolidone copolymer, hydroxypropyl cellulose, hydroxypropyl methylcellulose, methylcellulose, hydroxyethyl cellulose, sodium carboxymethylcellulose, calcium carboxymethylcellulose, polyvinyl acetate polyethylene glycol co-polymer, starches, maltodextrins, sugars, sorbitol, mannitol, sucrose, gelatin, casein, natural gums, alginates, and mixtures thereof.

An alternate embodiment is a controlled release topiramate dosage form, in which the granule coating is water insoluble and preferably, is comprised of a compound selected from the group consisting of polyethers such as ethyl cellulose, polyesters such as polyvinyl acetate, cellulose acetate, cellulose acetate butyrate, polyacrylates such as ethyl acrylate methylmethacrylate copolymers and mixtures thereof. An embodiment is a topiramate dosage form, in which the granule coating is, in addition to the ingredients above, is additionally comprised of a compound selected from the group consisting of polyethylene glycol, polyoxyethylene-polyoxypropylene copolymer, hydroxypropyl cellulose, glycerin, citric acid esters such as triethyl citrate and acetyl triethylcitrate, and dibutyl sebacate.

Another embodiment of the invention is a controlled release controlled release dosage form, in which the granule coating is comprised of a single layer. Another embodiment is a topiramate dosage form, in which the single-layer coating is comprised of polyvinyl pyrrolidone or polyethylene oxide. Another embodiment is a topiramate dosage form, in which the single-layer coating is up to about 3% of the granule weight, or up to about 30% of the granule weight.

Another embodiment is a controlled release topiramate dosage form, in which the granule coating is comprised of a multiplicity of layers. A preferable embodiment is a topiramate dosage form, in which the coating comprises two layers. A more preferable embodiment is a topiramate dosage form, in which the coating is comprised of an outer layer of methylcellulose and an inner layer of polyethylene oxide or polyvinyl pyrrolidone. Another embodiment is a topiramate dosage form, comprising an expandable layer, in which the semipermeable wall is positioned over at least a portion of the expandable layer.

An embodiment of the invention is a controlled release topiramate dosage form, in which the granule substrate comprises both topiramate and a soluvbilizing agent, preferably a surfactant. Another embodiment is a controlled release topiramate dosage form, in which the granule substrate further comprises a viscosity agent. Another embodiment is a controlled release topiramate dosage form, in which the granule substrate further comprises a binding agent.

Another embodiment is a controlled release topiramate dosage form, in which the release rate of topiramate is at its maximum value for at least two hours in an in vitro release rate assay. Additional embodiments are controlled release topiramate dosage forms, in which the release rate of topiramate is at its maximum value for at least about three hours, about four hours, about six hours, about eight hours, about ten hours and at least about 12 hours in an in vitro release rate assay.

In additional embodiments of the invention are controlled release topiramate dosage forms, in which the maximum release rate of topiramate is reached after about 1 hour, about 1.5 hours, about 2 hours, about 2.5 hours, about 3 hours, about 4 hours, about 5 hours, about 6.5 hours, about 8 hours, about 10 hours, about 12 hours, about 14 hours and about 16 hours in an in vitro release rate assay.

In one embodiment of the present invention is a drug composition comprising granules having a substrate and a coating, said granule substrate comprising a low solubility therapeutic drug and optionally a solubilizing agent, and said granule coating comprising one or more layers, wherein each granule coating layer comprises an independently selected hydrophilic polymer and wherein the drug composition possesses a dissolution rate of the therapeutic drug, a time to maximum release of the therapeutic drug and a period of therapeutic drug delivery (when the drug composition is tested in an in vitro assay or administered to a subject).

In another embodiment of the present invention is a drug composition comprising granules having a substrate and a coating, said granule substrate comprising a low solubility therapeutic drug and optionally a solubilizing agent, and said granule coating comprising one or more layers, wherein each granule coating layer comprises an independently selected hydrophilic polymer and wherein the drug composition when subjected to a measurement of dissolution exhibits a a dissolution rate of the therapeutic drug, a time to maximum release of the therapeutic drug and a period of therapeutic drug delivery.

In an embodiment of the present invention is a dosage form comprising
(a) a core comprising a drug composition, wherein the drug compositions comprise granules having a substrate and a coating, said granule substrate comprising a low solubility therapeutic drug and optionally a solubilizing agent, and said granule coating comprising one or more layers, wherein each granule coating layer comprises an independently selected hydrophilic polymer;
(b) a semi-permeable wall surrounding the core; and
(c) an exit orifice through the semi-permeable wall for releasing the drug compositions from the dosage form over a prolonged period of time.

In an embodiment of the present invention is a dosage form wherein the number of granule coating layers is selected to decrease the dissolution rate of the therapeutic drug or to increase the time to maximum release rate of the therapeutic drug or to increase the period of therapeutic drug delivery or decrease the magnitude of the maximum release rate of the therapeutic drug.

In another embodiment of the present invention is a dosage form wherein the granule coating comprises one layer and wherein the hydrophilic polymer is selected to decrease the dissolution rate of the therapeutic drug or increase the time to maximum release rate of the therapeutic drug or increase the period of therapeutic drug delivery or decrease the magnitude of the maximum release rate of the therapeutic drug.

In another embodiment of the present invention is a dosage form wherein the granule coating comprises one layer and wherein the amount of said coating is selected decrease the dissolution rate of the therapeutic drug or increase the time to maximum release rate of the therapeutic drug or increase the period of therapeutic drug delivery or decrease the magnitude of the maximum release rate of the therapeutic drug.

In another embodiment of the present invention is a dosage form wherein the the dissolution rate of the therapeutic drug or the time to maximum release rate of the therapeutic drug or the period of therapeutic drug delivery or the magnitude of the maximum release rate of the therapeutic drug is controlled by varying one or more of (a) the number of coating granule layers, (b) the amount of each coating granule layer or (c) the hydrophilic polymer of each coating granule layer.

In an embodiment of the present invention is a dosage form wherein the therapeutic drug is topiramate, wherein the granule coating comprises one layer and wherein the hydrophillic polymer is selected from the group consisting of methylcellulose and polyvinylpyrolidone.

In an embodiment of the present invention is a dosage form wherein the amount of granule coating is in the range of between about 2.5% and about 23% by weight. In another embodiment of the present invention is a dosage form wherein the amount of granule coating is in the range of between about 2.5% and about 23% by weight.

In an embodiment of the present invention is a dosage form wherein the granule coating comprises one layer and the hydrophilic polymer is selected from the group consisting of (a) methylcellulose in an amount in the range of between about 2.5% and about 13% by weight, (b) polyvinylpyrolidone in an amount in the range of between about 3% and about 23% by weight and (c) polyethylene oxide in an amount in the range of about 5% to about 15% by weight.

In another embodiment of the present invention is a dosage form wherein the granule coating comprises one layer and the hydrophilic polymer is methylcellulose present in an amount of about 3% by weight.

Another present invention is a method for controlling release patterns of a low solubility therapeutic drug, preferably topiramate, from a controlled release dosage form, comprising:
(a) preparing granule substrates comprising topiramate and a solubilizing agent, preferably surfactant,
(b) coating the granule substrates,
(c) mechanically compressing the coated granules to produce a tablet core,
(d) mechanically compressing the tablet core with a push layer and
(e) surrounding the tablet core and push layer by a semipermeable membrane having a delivery port across the membrane.

A preferred embodiment of the invention is a method for controlling the release patterns of topiramate, in which preparing coated granules further comprises:
(a) mixing topiramate with a solubilizing agent, preferably surfactant, and a solvent,
(b) forming a damp mass,
(c) drying the mass,
(d) sieving the dried mass to form granule substrates,
(e) transferring the granule substrates to a fluid bed granulator, and
(f) spray-coating the granule substrates with a spray-coating mixture to produce coated granules.

Another embodiment of the invention is a method for controlling release patterns of topiramate, in which the content and amount of the granule coating is selected to increase the amount of time to reach the maximum release rate of topiramate in an in vitro release rate assay. An alternate embodiment is a method of selecting the content and amount of the granule coating to modulate the period of time that the release rate of topiramate is at its maximum value in an in vitro release rate assay. Another embodiment is a method selecting the content and amount of the granule coating to modulate the magnitude of the maximum release rate of topiramate in an in vitro release rate assay.

Another embodiment of the invention is a method for controlling release patterns of topiramate, in which the weight ratio of the solubilizng agent, preferably, surfactant, to topiramate in the granule substrate is selected to modulate the amount of time that the release rate of topiramate is at its maximum value in an in vitro release rate assay. Alternate embodiments are methods of modifying the weight ratio of the solubilizing agent, preferably, surfactants to topiramate in the granule substrate to modulate the period of time that the release rate of topiramate is at its maximum value release patterns of topiramate, or the magnitude of the maximum release rate of topiramate in an in vitro release rate assay.

An additional embodiment of the invention is a method for controlling release patterns of topiramate, comprising the additional step of spraying-coating the coated granules with a second spray-coating mixture to produce coated granules having an outer coating layer and an inner coating layer. A related embodiment is a method of selecting the content and amount of the outer coating layer of the granule coating to increase the amount of time to reach the maximum release rate of topiramate in an in vitro release rate assay. Alternate embodiments are methods of modifying the content and amount of the outer coating layer of the granule coating to modulate the period of time that the release rate of topiramate is at its maximum value or the magnitude of the maximum release rate of topiramate in an in vitro release rate assay.

The present invention is further directed to a drug composition comprising a pharmaceautical agent and a solubilizing agent, wherein the pharmaceutical agent is selected from a low solubility pharmaceutical agent or a low dissolution rate pharmaceutical agent, wherein the pharmaceutical agent comprises greater than 11% by weight of the drug composition, wherein the solubilizing agent is a surfactant, and wherein the surfactant comprises greater than about 10% by weight of the drug composition.

The present invention is further directed to a drug composition comprising a pharmaceautical agent and a solubilizing agent, wherein the pharmaceutical agent is selected from a low solubility pharmaceutical agent or a low dissolution rate pharmaceutical agent, wherein the pharmaceutical agent comprises greater than 11% by weight of the drug composition and wherein the solubilizing agent is a surfactant.

In an embodiment of the present invention is a drug composition comprising a pharmaceutical agent, a solubilizing agent and a structural polymer, wherein the pharmaceutical agent is selected from a low solubility pharmaceutical agent or a low dissolution rate pharmaceutical agent, and wherein the pharmaceutical agent comprises greater than 11% by weight of the drug composition.

The present invention is further directed to a drug composition comprising topiramate and a solubilizing agent. In an embodiment of the present invention, the topiramate comprises greater than 11% by weight of the drug composition. In another embodiment of the present invention is a drug composition comprising topiramate, a solubilizing agent and a structural polymer. Preferably, the solubilizing agent is a surfactant. Preferably, the solubilizing agent comprises greater than about 10% by weight of the drug composition.

In an embodiment of the present invention is a drug composition comprising topiramate and a solubilizing agent, wherein the topiramate comprises greater than 11% by weight of the drug composition, wherein the solubilizing agent is a surfactant, and wherein the surfactant comprises greater than about 10% by weight of the drug composition.

In an embodiment of the present invention is a drug composition, wherein the pharmaceutical agent, preferably topiramate, comprises greater than about 20% by weight of the drug composition. Preferably, the pharmaceutical agent, preferably topiramate, comprises greater than about 30% by weight of the drug composition, more preferably, the pharmaceutical agent, preferably topiramate, comprises greater than about 40% by weight of the drug composition.

In another embodiment of the present invention is a drug composition, wherein the pharmaceutical agent, preferably topiramate, comprises between about 25% and about 55% by weight of the drug composition. Preferably, the pharmaceutical agent, preferably topiramate, comprises between about 30% and about 50% by weight of the drug composition.

In an embodiment of the present invention is a drug composition, wherein the solubilizing agent is a surfactant. In another embodiment of the present invention is a drug composition, wherein the solubilizing agent, preferably surfactant, comprises about 10% by weight of the drug composition, preferably, about 20% by weight of the drug composition, more prefereably, about 30% by weight of the drug composition, most preferably, about 40% by weight of the drug composition.

In another embodiment of the present invention is a drug composition, wherein the solubilizing agent, preferably a surfactant, comprises between about 35% and about 55% by weight of the drug composition. Preferably, the solubilizing agent, preferably surfactant, comprises between about 40% and about 50% by weight of the drug composition.

In an embodiment fo the present invention, the solubilizing agent is present in an amount greater than about 5%, more preferably, in an amount greater than about 10%, more preferably still, in an amount greater than about 17.5%, more preferably still, in an amount greater than about 25%, more preferably still, in an amount greater than about 30%, more preferably still, in an amount greater than about 40%, more preferably still, in an amount greater than about 42.5%, more preferably still, in an amount greater than about 45%.

In another embodiment of the present invention is a drug composition further comprising a structural polymer. Preferably, the structural polymer comprises between about 1% and about 90% by weight of the drug composition, preferably, the structural polymer comprises between about 5% and about 75% by weight of the drug composition, more preferably, the structural polymer comprises between about 10% and about 40% by weight of the drug composition.

The present invention is further directed to a dosage form comprising any of the drug compositions or formulations described herein. In an embodiment of the present invention is a dosage form comprising a drug composition, wherein the drug composition comprises topiramate and a solubilizing agent.

In an embodiment of the present invention, the dosage form is a matrix form. In another embodiment of the present invention, the dosage form is an osmotic dosage form. In another embodiment of the present invention, the dosage form is a controlled release dosage form. Preferably, the dosage form is a controlled release, osmotic dosage form, preferably for oral administration.

In an embodiment of the present invention is a dosage form comprising a drug composition as described herein, wherein the pharmaceutical agent is present in an amount in the range of about 1 milligram to about 750 milligrams, preferably about 5 milligrams to about 250 milligramns, more preferably about 10 milligrams to about 250 milligrams. In another embodiment of the present invention is a dosage form comprising two drug compositions as described herein, wherein the sum of the amount of pharmaceutical agent present within the drug compositions is in the range of about 1 milligram to about 750 milligrams, preferably about 5 milligrams to about 250 milligramns, more preferably about 10 milligrams to about 250 milligrams.

In another embodiment of the present invention is a dosage form comprising a drug composition, wherein the drug composition comprises topiramate, and a solubilizing agent, and wherein the topiramate is present in an amount in the range of about 1 milligram to about 750 milligrams, preferably about 5 milligrams to about 250 milligrams, more preferably about 10 milligrams to about 250 milligrams, more preferably still, the topiramate is present in an amount selected from 10 mg, 20 mg, 40 mg, 45 mg, 80 mg, 90 mg, 120 mg, 135 mg, 160 mg, 180 mg or 200 mg.

In another embodiment of the present invention is a dosage form comprising two drug compositions, wherein each drug composition comprises topiramate and an independently selected solubilizing agent, preferably surfactant, and wherein the sum of the amount of topiramate with the drug compositions is in the range of about 1 milligram to about 750 milligrams, preferably about 5 milligrams to about 250 milligramns, more preferably about 10 milligrams to about 250 milligrams, more preferably still, the topiramate is present in an amount selected from 10 mg, 20 mg, 40 mg, 45 mg, 80 mg, 90 mg, 120 mg, 135 mg, 160 mg, 180 mg or 200 mg.

In an embodiment of the present invention is a dosage form comprising (a) a core comprising a first drug composition and a push layer comprising an osmopolymer; (b) a semi-permeable wall surrounding the core; and (c) an exit orifice through the semi-permeable wall for releasing the drug compositions from the dosage form over a prolonged period of time.

In another embodiment of the present invention is a dosage form comprising (a) a core comprising a first drug composition, a second drug compsition and a push layer comprising an osmopolymer; (b) a semi-permeable wall surrounding the core; and (c) an exit orifice through the semi-permeable wall for releasing the drug compositions from the dosage form over a prolonged period of time.

In another embodiment of the present invention is a dosage form comprising (a) a core comprising a first drug composition, a second drug compsition and a push layer, wherein the first and second drug compsitition comprise topiramate and independently selected solubilizing agents; (b) a semi-permeable wall surrounding the core; and (c) an exit orifice through the semi-permeable wall for releasing the drug compositions from the dosage form over a prolonged period of time.

In another embodiment of the present invention is a dosage form comprising (a) a core comprising a first drug composition comprising a pharmaceutical agent and a solubilizing agent wherein the pharmaceutical agent is selected from a low solubility pharmaceutical agent or a low dissolution rate pharmaceutical agent, preferably topiramate, wherein the pharmaceutical agent comprises greater than 11% by weight of the drug composition, wherein the solubilizing agent is a surfactant, and wherein the surfactant comprises greater than about 10% by weight of the drug composition; a second drug composition comprising a pharmaceutical agent and a solubilizing agent wherein the pharmaceutical agent is selected from a low solubility pharmaceutical agent or a low dissolution rate pharmaceutical agent, wherein the pharmaceutical agent comprises greater than 11% by weight of the drug composition, wherein the solubilizing agent is a surfactant, and wherein the surfactant comprises greater than about 10% by weight of the drug composition; and a push layer, (b) a semi-permeable wall surrounding the core; and (c) an exit orifice through the semi-permeable wall for releasing the drug compositions from the dosage form over a prolonged period of time

In an embodiment of the present invention, the pharmaceutical agent and solubilizing agent in the first and second drug compositions are independently selected. Preferably, the pharmaceutical agent in the first and second drug compositions is the same, more preferably, the pharmaceutical agent in the first and second drug compositions is topiramate.

In an embodiment of the present invention, the amount and/or concentration of the pharmaceutical agent, preferably topiramate, within the first drug composition is less than the amount and/or concentration of the pharmaceutical agent, preferably topiramate, within the second drug composition.

In another embodiment of the present invention is a dosage form comprising (a) a core comprising a first drug composition, a second drug composition and a push layer comprising an osmopolymer; (b) a semi-permeable wall surrounding the core; and (c) an exit orifice through the semi-permeable wall for releasing the first drug composition and the second drug composition from the dosage form over a prolonged period of time; wherein the first drug composition comprises between about 25% and about 40% by weight of topiramate and between about 35% and about 50% by weight of a surfactant, and the second drug composition comprises between about 30% and about 40% by weight of topiramate and between about 45% and 55% by weight of a surfactant. In a preferred embodiment of the present invention, the first drug composition further comprises between about 10% and about 20% by weight of a structural polymer, and the second drug composition further comprises between about 0% and about 10% by weight of a structural polymer. Preferably, the first drug composition comprises between about 30% and about 35% by weight of topiramate, between about 40% and about 45% by weight of the surfactant, and between about 15% and about 20% by weight of the structural polymer, and the second drug composition comprises between about 40% and about 45% by weight of topiramate, between about 46% and about 54% by weight of the surfactant, and between about 0% and about 5% by weight of the structural polymer. More preferably, the first drug composition comprises about 32% by weight of topiramate, about 42% by weight of the surfactant, and about 16% by weight of the structural polymer, and the second drug composition comprises about 43% by weight of topiramate, about 50% by weight of the surfactant, and about 0% by weight of the structural polymer. Preferably, the surfactant in both the first and second drug compositions is LUTROL F127 and the structural polymer in both the first and second drug compositions is POLYOX N80.

In another embodiment of the present invention is a dosage form comprising (a) a core comprising a first drug composition, a second drug composition and a push layer comprising an osmopolymer; (b) a semi-permeable wall surrounding the core; and (c) an exit orifice through the semi-permeable wall for releasing the first drug composition and the second drug composition from the dosage form over a prolonged period of time; wherein the first drug composition comprises between about 1% and about 25% by weight of topiramate and between about 1% and 35% by weight of a surfactant, and the second drug composition comprises between about 10% and about 25% by weight of topiramate and between about 10% and 35% by weight of a surfactant. In a preferred embodiment of the present invention, the first drug composition further comprises between about 75% and about 95% by weight of a structural polymer, and the second drug composition further comprises between about 65% and about 80% by weight of a structural polymer. Preferably, the first drug composition comprises between about 2% and about 8% by weight of topiramate, between about 1% and about 5% by weight of the surfactant, and between about 85% and about 90% by weight of the structural polymer, and the second drug composition comprises between about 10% and about 15% by weight of topiramate, between about 10% and about 15% by weight of the surfactant, and between about 70% and about 75% by weight of the structural polymer. More preferably, the first drug composition comprises about 5% by weight of topiramate, about 2% by weight of the surfactant, and about 89% by weight of the structural polymer, and the second drug composition comprises about 12% by weight of topiramate, about 12% by weight of the surfactant, and about 72% by weight of the structural polymer. Preferably, the surfactant in both the first and second drug compositions is LUTROL F127 and the structural polymer in both the first and second drug compositions is POLYOX N80.

In an embodiment of the present invention, the push layer comprises an osmopolymer. In another embodiment of the present invention, the push layer comprises an osmopolymer and an osmoagent.

In an embodiment of the present invention, the dosage form releases drug over a prolonged period of time, preferably over greater than 4 hours, more preferably, over greater than about 8 hours, more preferably still, over greater than about 10 hours, most preferably, over greater than about 14 hours. In another embodiment of the present invention, the dosage form releases drug over a prolonged period of time greater than about 14 hours and up to about 24 hours.

In an embodiment of the present invention, the dosage form releases drug with a substantially ascending rate of release. In another embodiment of the present invention, the dosage form releases drug with a substantially ascending rate of release. In yet another embodiment of the present invention, the dosage form releases drug at a rate which results in a substantially ascending drug plasma concentration.

In an embodiment of the present is a drug composition comprising topiramate, a surfactant, preferably LUTROL F127 and a structural polymer, preferably POLYOX N80; wherein the topiramate comprises about 5% by weight of the drug composition, wherein the surfactant comprises about 2% by weight of the drug composition, and wherein the structural polymer comprises about 88.7% by weight of the drug composition.

In an embodiment of the present is a drug composition comprising topiramate, a surfactant, preferably LUTROL F127 and a structural polymer, preferably POLYOX N80; wherein the topiramate comprises about 12% by weight of the drug composition, wherein the surfactant comprises about 12% by weight of the drug composition, and wherein the structural polymer comprises about 71.7% by weight of the drug composition.

In an embodiment of the present is a drug composition comprising topiramate, a surfactant, preferably LUTROL F127 and a structural polymer, preferably POLYOX N80; wherein the topiramate comprises about 32% by weight of the drug composition, wherein the surfactant comprises about 42% by weight of the drug composition, and wherein the structural polymer comprises about 16.5% by weight of the drug composition.

In an embodiment of the present is a drug composition comprising topiramate, and a surfactant, preferably LUTROL F127; wherein the topiramate comprises about 43% by weight of the drug composition, and wherein the surfactant comprises about 49.9% by weight of the drug composition.

In an embodiment of the present invention is a drug composition comprising topiramate, wherein the topiramate comprises about 5% by weight of the drug composition; surfactant, preferably LUTROL F127 wherein the surfactant comprises about 2% by weight of the drug composition; a structural polymer, preferably POLYOX N80 wherein the structural polymer comprises about 88.7% by weight of the drug composition; PVP, preferably PVP K29-32, wherein the PVP comprises about 3% by weight of the drug composition; stearic acid, wherein the stearic acid comprises about 1% by weight of the drug composition; magnesium stearate, wherein the magnesium stearate comprises about 0.25% by weight of the drug composition; and butylated hydroxytoluene (BHT), wherein the BHT comprises about 0.02% by weight of the drug composition.

In an embodiment of the present invention is a drug composition comprising topiramate, wherein the topiramate comprises about 12% by weight of the drug composition; surfactant, preferably LUTROL F127, wherein the surfactant comprises about 12% by weight of the drug composition; a structural polymer, preferably POLYOX N80, wherein the structural polymer comprises about 71.7% by weight of the drug composition; PVP, preferably PVP K29-32, wherein the PVP comprises about 3% by weight of the drug composition; stearic acid, wherein the stearic acid comprises about 1% by weight of the drug composition; magnesium stearate, wherein the magnesium stearate comprises about 0.25% by weight of the drug composition; iron oxide, wherein the iron oxide comprises about 0.02% by weight of the drug composition, and BHT, wherein the BHT comprises about 0.02% by weight of the drug composition.

In an embodiment of the present invention is a drug composition comprising topiramate, wherein the topiramate comprises about 32% by weight of the drug composition; surfactant, preferably LUTROL F127, wherein the surfactant comprises about 42% by weight of the drug composition; a structural polymer, preferably POLYOX N80, wherein the structural polymer comprises about 16.5% by weight of the drug composition; PVP, preferably PVP K29-32, wherein the PVP comprises about 3% by weight of the drug composition; stearic acid, wherein the stearic acid comprises about 1% by weight of the drug composition; magnesium stearate, wherein the magnesium stearate comprises about 0.5% by weight of the drug composition; BHT, wherein the BHT comprises about 0.02% by weight of the drug composition and methylcellulose, wherein the methylcellulose comprises about 2.5% by weight of the drug composition.

In an embodiment of the present invention is a drug composition comprising topiramate, wherein the topiramate comprises about 43% by weight of the drug composition; surfactant, preferably LURTOL F127, wherein the surfactant comprises about 49.9% by weight of the drug composition; PVP, preferably PVP K29-32, wherein the PVP comprises about 3% by weight of the drug composition; stearic acid, wherein the stearic acid comprises about 1% by weight of the drug composition; magnesium stearate, wherein the magnesium stearate comprises about 0.5% by weight of the drug composition; ferric oxide, wherein the ferric oxide comprises about 0.08% by weight of the drug composition; BHT, wherein the BHT comprises about 0.02% by weight of the drug composition and methylcellulose, wherein the methylcellulose comprises about 2.5% by weight of the drug composition.

In an embodiment of the present invention is a dosage form comprising (a) a core comprising a first drug composition comprising topiramate, a surfactant, preferably LUTROL F127 and a structural polymer, preferably POLYOX N80 wherein the topiramate comprises about 5% by weight of the drug composition, wherein the surfactant comprises about 2% by weight of the drug composition, and wherein the structural polymer comprises about 88.7% by weight of the drug composition; a second drug composition comprising topiramate, a surfactant, preferably LUTROL F127 and a structural polymer, preferably POLYOX N80 wherein the topiramate comprises about 12% by weight of the drug composition, wherein the surfactant comprises about 12% by weight of the drug composition, and wherein the structural polymer comprises about 71.7% by weight of the drug composition; and a push layer.

In another embodiment of the present invention is a dosage form comprising (a) a core comprising a first drug composition comprising topiramate, a surfactant, preferably LUTROL F127 and a structural polymer, preferably POLYOX N80; wherein the topiramate comprises about 32% by weight of the drug composition, wherein the surfactant comprises about 42% by weight of the drug composition, and wherein the structural polymer comprises about 16.5% by weight of the drug composition; a second drug composition comprising topiramate, and a surfactant, preferably LUTROL F127; wherein the topiramate comprises about 43% by weight of the drug composition, and wherein the surfactant comprises about 49.9% by weight of the drug composition; and a push layer.

In an embodiment of the present invention is a dosage form comprising (a) a core comprising a first drug composition comprising topiramate, wherein the topiramate comprises about 5% by weight of the drug composition; surfactant, preferably LUTROL F127 wherein the surfactant comprises about 2% by weight of the drug composition; a structural polymer, preferably POLYOX N80, wherein the structural polymer comprises about 88.7% by weight of the drug composition; PVP, preferably PVP K29-32, wherein the PVP comprises about 3% by weight of the drug composition; stearic acid, wherein the stearic acid comprises about 1% by weight of the drug composition; magnesium stearate, wherein the magnesium stearate comprises about 0.25% by weight of the drug composition; and BHT, wherein the BHT comprises about 0.02% by weight of the drug composition; a second drug composition comprising topiramate, wherein the topiramate comprises about 12% by weight of the drug composition; surfactant, preferably LUTROL F127 wherein the surfactant comprises about 12% by weight of the drug composition; a structural polymer, preferably POLYOX N80, wherein the structural polymer comprises about 71.7% by weight of the drug composition; PVP, preferably PVP K29-32, wherein the PVP comprises about 3% by weight of the drug composition; stearic acid, wherein the stearic acid comprises about 1% by weight of the drug composition; magnesium stearate, wherein the magnesium stearate comprises about 0.25% by weight of the drug composition; iron oxide, wherein the iron oxide comprises about 0.02% by weight of the drug composition, and BHT, wherein the BHT comprises about 0.02% by weight of the drug composition; and a push layer comprising an osmopolymer; (b) a semi-permeable wall surrounding said core; and (c) an exit orifice through the semi-permeable wall for releasing the first drug composition and the second drug composition from the dosage form over a prolonged period of time.

In another embodiment of the present invention is a dosage form comprising (a) a core comprising a first drug composition comprising topiramate, wherein the topiramate comprises about 32% by weight of the drug composition; surfactant, preferably LUTROL F127, wherein the surfactant comprises about 42% by weight of the drug composition; a structural polymer, preferably POLYOX N80, wherein the structural polymer comprises about 16.5% by weight of the drug composition; PVP, preferably PVP K29-32, wherein the PVP comprises about 3% by weight of the drug composition; stearic acid, wherein the stearic acid comprises about 1% by weight of the drug composition; magnesium stearate, wherein the magnesium stearate comprises about 0.5% by weight of the drug composition; BHT, wherein the BHT comprises about 0.02% by weight of the drug composition and methylcellulose, wherein the methylcellulose comprises about 2.5% by weight of the drug composition; a second drug composition comprising topiramate, wherein the topiramate comprises about 43% by weight of the drug composition; surfactant, preferably LUTROL F127, wherein the surfactant comprises about 49.9% by weight of the drug composition; PVP, preferably PVP K29-32, wherein the PVP comprises about 3% by weight of the drug composition; stearic acid, wherein the stearic acid comprises about 1% by weight of the drug composition; magnesium stearate, wherein the magnesium stearate comprises about 0.5% by weight of the drug composition; ferric oxide, wherein the ferric oxide comprises about 0.08% by weight of the drug composition; BHT, wherein the BHT comprises about 0.02% by weight of the drug composition and methylcellulose, wherein the methylcellulose comprises about 2.5% by weight of the drug composition; and a push layer comprising an osmopolymer; (b) a semi-permeable wall surrounding said core; and (c) an exit orifice through the semi-permeable wall for releasing the first drug composition and the second drug composition from the dosage form over a prolonged period of time.

In an embodiment of the present invention is a method of treating a disorder selected from the group consisting of epilepsy, migraine, glaucoma and other ocular disorders (including diabetic retinopathy), essential tremor, restless limb syndrome, obesity, weight loss, Type II Diabetes Mellitus, Syndrome X, impaired oral glucose tolerance, diabetic skin lesions, cluster headaches, neuralgia, neuropathic pain (including diabetic neuropathy), elevated blood glucose levels, elevated blood pressure, elevated lipids, bipolar disorder, dementia, depression, psychosis, mania, anxiety, schizophrenia, OCD, PTSD, ADHD, impulse control disorders (including bulimia, binge eating, substance abuse, etc.), ALS, asthma, autism, autoimmune disorders (including psoriasis, rheumatoid arthritis, etc.), chronic neurodegenerative disorders, acute neurodegeneration, sleep apnea and other sleep disorders and/or for promoting wound healing, comprising administering to a subject in need thereof, of any of the drug compositions or dosage forms described herein.

Preferably, the disorder is selected from the group consisting of epilepsy, migraine, diabetic retinopathy, diabetic neuropathy, diabetic skin lesions, obesity, weight loss, Type II Diabetes Mellitus, Syndrome X, impaired oral glucose tolerance, elevated blood glucose levels and elevated blood pressure.

### BRIEF DESCRIPTION OF THE DRAWINGS

FIG. 1 shows smearing of a formulation compressed in the absence of coated granules.

The following six figures (FIGS. 2 to 7) are not drawn to scale, and are set forth to illustrate various embodiments of the invention.

FIG. 2 illustrates an embodiment of an osmotic dosage form of the present invention, illustrating the dosage form prior to administration to a subject.

FIG. 3 illustrates the dosage form of FIG. 2 in opened section, illustrating a single internally housed drug composition.

FIG. 4 illustrates the dosage form of FIG. 2 in opened section view, illustrating a bi-layer comprising a drug composition and a separate and contacting push layer for pushing the drug composition from the dosage form.

FIG. 5 illustrates the dosage form of FIG. 2, which further comprises an immediate release external overcoat of pharmaceutical agent on the dosage form.

FIG. 6 illustrates an opened view of another embodiment of the dosage form of the present invention illustrating a tri-layer arrangement comprising two drug compositions in parallel arrangement and a separate and contacting push layer for pushing the drug layers from the capsule shaped dosage form.

FIG. 7 shows release of drug from a controlled release dosage form consisting of a push-stick system with coated granules.

FIG. 8 shows topiramate release patterns of control release topiramate dosage forms having coated granules in a 1 kg (upper panel) and 30 kg (lower panel) batch size.

FIG. 9 shows the topiramate release from control release topiramate dosage forms. In all dosage forms, the granules were coated with 23% PVP. Top panel: 0% methylcellulose granule coating; bottom panel: 3% methylcellulose granule coating.

FIG. 10 shows delivery patterns of topiramate dosage forms as a function of the LUTROL F127/topiramate (surfactant/drug) weight ratios in the coated granules. From top to bottom panels: 1.86 surfactant/drug; 1.49 surfactant/drug; 1.18 surfactant/drug; 0.93 surfactant/drug.

FIG. 11 shows delivery patterns of topiramate dosage forms as a function of coating content. Top panel: 23% PVP with 3% methylcellulose bilayer coating; bottom panel 10% POLYOX N10 single layer coating.

FIG. 12 shows smear resistance and delivery patterns of topiramate dosage forms as a function of coating thickness. Top to bottom panels: 3% methylcellulose; 7% methylcellulose; 13% methylcellulose.

FIG. 13 shows smear resistance and delivery patterns of topiramate dosage forms as a function of coating content. Top panel: single layer, 3% methylcellulose; bottom panel: bilayer, 10% PVP with 3% methylcellulose granule coating.

FIG. 14 shows delivery patterns of topiramate dosage forms as a function of coating content. All dosage form coatings have 10% PVP. Top to bottom panels: 3% methylcellulose; 7% methylcellulose; 10% methylcellulose granule coating.

### DETAILED DESCRIPTION OF THE INVENTION

### Terminology

The present invention is best understood by reference to the following definitions, the drawings and exemplary disclosure provided herein.

To provide a more concise description, some of the quantitative expressions given herein are not qualified with the term “about”. It is understood that whether the term “about” is used explicityly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

The expressions “exit”, “exit orifice” and “delivery port” shall mean an opening in a dosage form which permits drug to exit the dosage form. Suitable examples include, but are not limited to, a passageway; an aperture; an orifice; and a bore. The expressions also include an orifice that is formed or formable from a substance or polymer that erodes, dissolves or is leached from the outer wall to thereby form an exit orifice.

By “dosage form” is meant a pharmaceutical composition or device capable of delivering a pharmaceutical agent. Suitable examples of dosage forms include, but are not limited to tablets, capsules, gel-caps, matrix forms, osmotic forms, immediate release forms, controlled release forms, sustained release forms, extended release forms, and the like. Similarly, the term “tablet core” shall mean any drug composition or formulation which comprises that portion of the dosage form which comprises the therapeutic or pharmaceutical agent. The tablet core may be incoprprated into any suitable dosage form, including, but not limited to a tablet, capsule, gel cap, and the like. Further, the tablet core may optionally be surrounded, partially or completely, by an outer layer, for example a semi-permeable membrane wall, hard shell capsule, and the like. The tablet core may be further optionally compressed or constituted by filling or the like.

As used herein, unless otherwise noted, the terms “drug composition” and “drug formulation” shall mean a formulation comprising at least one pharmaceutical agent. Preferably, the drug composition comprises a pharmaceutical agent and a solubilizing agent, preferably, a surfactant, more preferably a solubilizing surfactant. More preferably, the drug composition comprises a pharmaceutical agent, a solubilizing agent, preferably, a surfactant and a structural polymer. The drug composition may further optionally contain one or more inactive ingredients, i.e., pharmaceutically acceptable excipients such as disintegrants, binders, diluents, lubricants, stabilizers, antioxidants, osmotic agents, colorants, plasticizers, coatings and the like.

As used herein, unless otherwise noted, the term “push layer” shall mean a formulation which does not contain pharmaceutical agent and which comprises an osmopolymer. Preferably, the push layer comprises an osmopolymer and an osmoagent. The push layer may further optionally contain one or more inactive ingredients, for example disintegrants, binders, diluents, lubricants, stabilizers, antioxidants, osmotic agents, colorants, plasticizers, coatings and the like. Similarly, the term “osmotic delivery system” shall mean any means for delivering a therapeutic or pharmaceutical agent which uses osmotic gradient to achieve such delivery, for example a push layer as described above.

As used herein, unless otherwise noted, the terms “pharmaceutical agent”, “therapeutic drug”, “therapeutic agent”, “drug compound” and “drug” shall mean a pharmaceutical agent, drug, compound, pharmaceutically acceptable salt, prodrug or derivative thereof. Preferably, the pharmaceutical agent or drug is a low solubility and/or low dissolution rate pharmaceutical agent. More preferably, the pharmaceutical agent is topiramate.

As used herein, unless otherwise noted, the term “pharmaceutically acceptable salt”, shall mean any salt whose anion or cation does not contribute significantly to the toxicity or pharmacological activity of the salt, and, as such, they are the pharmacological equivalents of the acids or bases of the compound. Suitable pharmaceutically acceptable salts include acid addition salts which may, for example, be formed by reacting the drug compound with a suitable pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid; and base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base.

Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases which may be used in the preparation of pharmaceutically acceptable salts include the following: acids including acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydrocy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitric acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

As used herein the term “low solubility” shall mean that the neat pharmaceutical agent (in the absence of surfactants or other excipients) exhibits a solubility of less than about 100 mg/ml in de-ionized water at 37° C. Preferably, low solubility shall mean a solubility of less than about 50 mg/ml, more preferably, less than about 25 mg/ml, more preferably still, less than about 15 mg/ml, more preferably still, less than about 10 mg/ml, more preferably still, less than about 5 mg/ml, most preferably, less than about 1 mg/ml. Similarly, the term “lowly soluble” when used to characterize a drug shall therefore mean a drug that exhibits low solubility as herein defined.

As defined herein, the solubility of a pharmaceutical agent is determined by adding the pharmaceutical agent to stirred or agitated de-ionized water maintained in a constant temperature bath at a temperature of 37° C. until no more pharmaceutical agent dissolves. The resulting solution saturated with the pharmaceutical agent is then filtered, typically under pressure through a 0.8-micron Millipore filter, and the concentration of the pharmaceutical agent in the solution is measured by any appropriate analytical method including gravimetric, ultraviolet spectrophometry, chromatography, and the like. The solubility of the pharmaceutical agent is measured at equilibrium.

As used herein, the term “low dissolution rate” shall mean that rate of dissolution of the pharmaceutical agent under constant surface area (i.e the rate at which the pharmaceutical agent dissolves in de-ionized water at 37° C.) is between 0 mg/min/cm² and about 20 mg/min/cm², preferably, between about 0.1 mg/min/cm² and about 10 mg/min/cm², more preferably, between about 0.1 mg/min/cm² and about 5 mg/min/cm², more preferably still, between about 0.1 mg/min/cm² and about 2 mg/min/cm², more preferably still, between about 0.1 mg/min/cm² and about 1.5 mg/min/cm², most preferably, between about 0.1 mg/min/cm² and about 1.25 mg/min/cm².

As defined herein, the dissolution rate of a pharmaceutical agent is determined by the method as described in USP 26, NF21, p.2333.

Suitable examples of low solubility pharmaceutical agents (i.e. those with a solubility in de-ionized water at 37° C. of less than about 100 mg/ml) include, but are not limited to itraconazole, loratadine, thioridazine, thiethylperazine, ketoconazole, terfenadine, tretinoin, methdilazine, buprenorphine, thiothixene, simvastatin, indomethacin, domperidone, erythromycin, vitamin B, levonorgestrel, lovastatin, nicardipine, diclofenac, chlorpromazine, estradiol, digitoxin, liothyronine, glyburide, droperidol, verapamil, triazolam, fluocinonide, loxapine, prazepam, lindane, flurbiprofen, oxaprozin, progesterone, pimozide, methyclothiazide, ethinyl estradiol, finasteride, clozapine, haloperidol, diflunisal, prochloperazine, warfarin, imipramine, felodipine, mefenamic acid, methotrimeprazine, ibuprofen, spironolactone, nimodipine, biperiden, perphenazine, fluphenazine, methyltestosterone, glipizide, disopyramide, methoxsalen, diazepam, penicillin, ketoprofen, nifedipine, etoposide, metolazone, digoxin, betamethasone, fluoxymesterone, nabumetone, reserpine, furosemide, sulfadiazine, nitrendipine, nitrofurantoin, lorazepam, triamcinolone, omeprazole, dexamethasone, doxorubicin, clonazepam, bendroflumethiazide, chlorthalidone, methylprednisolone, pyrimethamine, flumazenil, tetracaine, fludrocortisone, quinidine, morphine, temazepam, oxazepam, epinephrine, fentanyl, cefazolin, prednisolone, tetracycline, chlorpropamide, chlorothiazide, azathioprine, prednisone, hydrocortisone, nystatin, phenazopyridine, trimethoprim, fenfluramine, isosorbide dinitrate, allopurinol, sulfamethoxazole, doxycycline, hydrochlorothiazide, amphotericin B, diphenoxylate, trichlormethiazide, zidovudine, famotidine, and the like.

Preferably, the low solubility pharmaceutical agent is other than (is not) phenytoin. Preferably, the low solubility pharmaceutical agent is other than phenytoin and carbamazepine. Preferably, the low solubility pharmaceutical agent is other than phenytoin, mephenytoin, phenobarbital, primidone, carbamazepine, ethosuximide, methsuximide, phensuximide, trimethadione, clonazipam, clorazepate, phenacemide, paramethadione, primaclone, clobazam, felbamate, flunarizine, lamotrigine, progabide, vibabatim, eterobarb, gabapentin, oxcarbazepine, ralitoline, tiagobine, sulthiame and tioridone.

The low solubility and/or low dissolution rate pharmaceutical agents may be incorporated into the drug composition and/or dosage forms of the present invention in amounts in the range of from about 1 milligram to about 750 milligrams, preferably in the range of from about 5 mg to about 250 mg, more preferably in the range of from about 10 mg to about 250 mg.

An “immediate-release dosage form” refers to a dosage form that releases greater than or equal to about 80% of the pharmaceutical agent in less than or equal to about 1 hour.

By “sustained release” is meant continuous release of a pharmaceutical agent over a prolonged period of time.

By “controlled release” is meant continuous release of a pharmaceutical agent over a prolonged period of time, wherein the pharmaceutical agent is released at a controlled rate over a controlled period of time.

By “prolonged period of time” is meant a continuous period of time of greater than about 1 hour, preferably, greater than about 4 hours, more preferably, greater than about 8 hours, more preferably greater than about 10 hours, more preferably still, greater than about 14 hours, most preferably, greater than about 14 hours and up to about 24 hours.

As used herein, unless otherwise noted, “rate of release” or “release rate” of a drug refers to the quantity of drug released from a dosage form per unit time, e.g., milligrams of drug released per hour (mg/hr). Drug release rates for dosage forms are typically measured as an in vitro rate of drug release, i.e., a quantity of drug released from the dosage form per unit time measured under appropriate conditions and in a suitable fluid.

The release rates referred to herein are determined by placing a dosage form to be tested in de-ionized water in metal coil or metal cage sample holders attached to a USP Type VII bath indexer in a constant temperature water bath at 37° C. Aliquots of the release rate solutions, collected at pre-set intervals, are then injected into a chromatographic system fitted with an ultraviolet or refractive index detector to quantify the amounts of drug released during the testing intervals. The above described method is therefore an example of an in vitro release rate assay.

As used herein a drug release rate obtained at a specified time refers to the in vitro release rate obtained at the specified time following implementation of the release rate test. The time at which a specified percentage of the drug within a dosage form has been released from said dosage form is referred to as the “Tₓ” value, where “x” is the percent of drug that has been released. For example, a commonly used reference measurement for evaluating drug release from dosage forms is the time at which 70% of drug within the dosage form has been released. This measurement is referred to as the “T₇₀” for the dosage form. Preferably, T₇₀ is greater than or equal to about 8 hours, more preferably, T₇₀ is greater than or equal to about 12 hours, more preferably still, T₇₀ is greater than to equal to about 16 hours, most preferably, T₇₀ is greater than or equal to about 20 hours. Preferably, T₇₀ is less than about 24 hours, more preferably, T₇₀ is less than about 20 hours.

By “C” is meant the concentration of drug in blood plasma, or serum, of a subject, generally expressed as mass per unit volume, typically nanograms per milliliter. For convenience, this concentration may be referred to herein as “drug plasma concentration”, “plasma drug concentration” or “plasma concentration” which is intended to be inclusive of a drug concentration measured in any appropriate body fluid or tissue. The plasma drug concentration at any time following drug administration is referenced as Cₜᵢₘₑ, as in C₉ₕ or C₂₄ₕ, etc.

As used herein, “steady state” when used in describing the drug plasma concentration of a pharmaceutical agent, shall mean a plasma drug concentration in the range of from about 5 ng/ml to about 500 ng/ml, preferably, from about 25 ng/ml to about 250 ng/ml, with the proviso that during the 24 hour period after administration the quotient formed by [Cₘₐₓ−Cₘᵢₙ]/C_{avg} (i.e. the variation in the blood plasma concentration of the drug) is about 3 or less, preferably, about 2 or less, more preferably, about 1 or less.

Persons of skill in the art will appreciate that blood plasma drug concentrations obtained in individual subjects will vary due to interpatient variability in the many parameters affecting drug absorption, distribution, metabolism and excretion. For this reason, unless otherwise indicated, when a drug plasma concentration is listed, the value listed is the calculated mean value based on values obtained from a groups of subjects tested.

As used herein, unless otherwise noted, the term “zero order rate of release” shall mean a rate of release wherein the amount of drug released as a function of time is substantially constant. More particularly, the rate of release of drug as a function of time shall vary by less than about 30%, preferably, less than about 20%, more preferably, less than about 10%, most preferably, less than about 5%, wherein the measurement is taken over the period of time wherein the cumulative release is between about 25% and about 75%, preferably, between about 25% and about 90%.

As used herein unless otherwise noted, the term “ascending rate of release” shall mean a rate of release wherein the amount of drug released as a function of time increases over a period of time, preferably continuously and gradually. Preferably, the rate of drug released as a function of time increases in a steady (rather than step-wise) manner. More preferably, an ascending rate of release may be characterized as follows. The rate of release as a function of time for a dosage form is measured and plotted as % drug release versus time or as milligrams of drug released/hour versus time. An ascending rate of release is characterized by an average rate (expressed in mg of drug per hour) wherein the rate within a given two hour span is higher as compared with the previous two hour time span, over the period of time of about 2 hours to about 12 hours, preferably, about 2 hours to about 18 hours, more preferably about 4 hours to about 12 hours, more preferably still, about 4 hours to about 18 hours. Preferably, the increase in average rate is gradual such that less than about 30% of the dose is delivered during any 2 hour interval, more preferably, less than about 25% of the dose is delivered during any 2 hour interval. Preferably, the ascending release rate is maintained until at least about 50%, more preferably until at least about 75% of the drug in the dosage form has been released.

One skilled in the art will recognize that as the increase in the area under the curve increases (e.g from 1% to 10%), the total time over which the drug is released from the dosage form will necessarily decrease and as such the determination of ascending rate of release will span a shorter overall period of time.

As used herein, the term or “ascending drug plasma concentration” shall mean a drug plasma concentration profile over about the first 24 hours following initial dosing, wherein the profile shows an increase to a maximum concentration, wherein said maximum occurs more than about 6 hours following the initial dose, preferably, more than about 8 hours following initial dose, more preferably, more than about 12 hours after dose.

When referring to a drug composition, “high dosage” shall mean a drug composition wherein the pharmaceutical agent, preferably topiramate, is present in an amount greater than or equal to about 20%, preferably greater than or equal to about 30%, more preferably greater than or equal to about 40%, by weight of the total drug composition.

When referring to a dosage form, “high dosage” shall mean a dosage form wherein the pharmaceutical agent, preferably topiramate, is present in an amount greater than or equal to about 20%, preferably greater than or equal to about 30%, more preferably greater than or equal to about 40%, by weight of the drug compositions within the dosage form.

As used herein, the term “therapeutically effective amount” shall mean that amount of pharmaceutical agent that elicits the biological or medicinal response in a tissue system, animal or human that is being sought by a researcher, veterinarian, medical doctor or other clinician, which includes alleviation of the symptoms of the disease or disorder being treated.

The term “subject” as used herein, refers to an animal, preferably, a mammal, most preferably, a human, who has been the object of treatment, observation or experiment.

As used herein, unless otherwise noted, the term “structural polymer” shall mean any component, for example a polymer or sugar, which is capable of water absorption and which may increase the viscosity of the drug compositions and/or may impart osmotic activity to the drug composition and or may act as a suspending agent for the drug composition. Suitable examples of structural polymers include, but are not limited to poly(alkyleneoxide polymers of between 100,000 and 750,000 molecular weight, including polyethylene oxide (such as POLYOX® N80; POLYOX®N10, POLYOX N750, and the like); polymethylene oxide, polybutylene oxide and polyhexylene oxide, and poly(carboxymethylcellulose) of 40,000 to 400,000 number average molecular weight, represented by poly(alkali carboxymethylcellulose), poly(sodium carboxymethylcellulose), poly(potassium carboxymethylcellulose), poly(litihium carboxymethylcellulose), and the like. Suitable example also include, but are not limited to sugars such as maltrodextrins (such as MALTRIN M040, MALTRIN M100, MALTRIN M150, MALTRIN M200, MALTRIN M250, and the like); sugars comprising lactose, glucose, raffinose, sucrose, mannitol, sorbitol and the like. Suitable examples also include, but are not limited to polyvinylpyrrolidone (PVP) (such as PVPs of grades 12PF or K2932, and the like); hydroxypropylcellulose; hydroxy propyl alkylcellulose of 9200 to 125,000 average molecular weight represented by hydroxypropyl ethylcellulose, hydroxypropoyl methylcellulose, hydroxypropyl butylcellu lose, hydroxypropyl pentylcellulose, and the like; polyvinyl pyrrolisone vinyl acetate co-olymers; and poly(vinylpyrrolidone) of upto 1,000,000 average molecular weight. Preferably, the structural polymer is a polyethyleneoxide polymers of between 100,000 and 300,000 molecular weight. More preferably, the structural polymer is POLYOX®N80.

Preferably, the structural polymer is selected from MALTRIN M100, POLYOX N10 and POLYOX N80, more preferably, the structural polymer is POLYOX N80.

As used herein, unless otherwise noted, the term “solubilizing agent” shall mean any component which increases the solubility and/or dissolution rate of a pharmaceutical agent. Preferably, the solubilizing agent is a surfactant. Suitable examples of solubilizing agents include, but are not limited to polyethylene glycol (PEG) 3350, polyethylene glycol 8K, and surfactants including, but not limited to, KOLLIDON K90, KOLLIDON 12PF, KOLLIDON 17PF, KOLLIDON 25/30; LUTROL F68, LUTROL F87, LUTROL F127, LUTROL F108; MYRJ 52, MYRJ 53; PVP K2939, and the like. Additional preferred surfactants include, but are not limited to, sorbitan monopalmitate, sorbitan monostearate, glycerol monostearate, polyoxyethlene stearate, sucrose cocoate, polyoxyethylene 40 sorbitol lanolin derivative, polyoxyethylene 75 sorbitol lanolin derivative, polyoxyethylene 6 sorbitol beeswax derivative, polyoxyethylene 20 sorbitol beeswax derivative, polyoxyethylene 20 sorbitol lanolin derivative, polyoxyethylene 50 sorbitol lanolin derivative, polyoxyethylene 23 lauryl ether, polyoxyethylene 23 lauryl ether with butylated hydroxyanisole and citric acid added as preservatives, polyoxyethylene 2 cetyl ether with butylated hydroxyanisole and citric acid added as preservatives, polyoxyethylene 2 stearyl ether, polyoxyethylene 21 stearyl ether, polyoxyethylene 100 stearyl ether, polyoxyethylene 10 cetyl ether with butylated hydroxyanisole and citric acid added as preservatives, polyoxyethylene 20 cetyl ether with butylated hydroxyanisole and citric acid added as preservatives, polyoxyethylene 2 stearyl ether with butylated hydroxyanisole and citric acid added as preservatives, polyoxyethylene 10 stearyl ether with butylated hydroxyanisole and citric acid added as preservatives, polyoxyethylene 20 stearyl ether with butylated hydroxyanisole and citric acid added as preservatives, polyoxyethylene 21 stearyl ether with butylated hydroxyanisole and citric acid added as preservatives, polyoxyethylene 20 oleyl ether with butylated hydroxyanisole and citric acid added as preservatives, polyoxyethylene 40 stearate, polyoxyethylene 50 stearate, polyoxyethylene 100 stearate, sorbitan monopalmitate, sorbitan monostearate, sorbitan tristearate, polyoxyethylene 4 sorbitan monostearate, polyoxyethylene 20 sorbitan tristearate

More preferably, the solubilizing agent is a surfactant selected form the group of co-polymers of ethylene oxide and propylene oxide conforming to the general formula OH(C₂H₄O)ₐ(C₃H₆O)_{b}(C₂H₄O)H. More preferably still, the surfactant is selected from the group consisting of LUTROL F68, LUTROL F87, LUTROL 108, LUTROL F127, MYRJ 52, MYRJ 53; most preferably, the surfactant is LUTROL F127.

As used herein, unless otherwise noted, the term “osmopolymer” shall mean a swellable, hydrophilic polymer that interacts with water and swells or expands to a high degree, typically exhibiting a 2-50 fold volume increase. Suitable examples, include but are not limited to poly(alkylene oxide) of 1 million to 15 million number-average molecular weight, as represented by poly(ethylene oxide), poly(alkali carboxymethylcellulose) of 500,000 to 3,500,000 number-average molecular weight, wherein the alkali is sodium, potassium or lithium; polymers that form hydrogels, such as CARBOPOL® acidic carboxypolymer, a polymer of acrylic cross-linked with a polyallyl sucrose, also known as carboxypolymethylene, and carboxyvinyl polymer having a molecular weight of 250,000 to 4,000,000; CYANAMER® polyacrylamides; cross-linked water swellable indenemaleic anhydride polymers; GOOD-RITE® polyacrylic acid having a molecular weight of 80,000 to 200,000; AQUA-KEEPS® acrylate polymer polysaccharides composed of condensed glucose units, such as diester cross-linked polygluran; and the like.

As used herein, unless otherwise noted, the terms “osmoagent”, “osmotic agent” and “osmotically active agent” shall mean an agent which exhibits an osmotic activity gradient across a semi-permeable membrane. Suitable osmoagents include, but are not limited to, sodium chloride, potassium chloride, lithium chloride, magnesium sulfate, magnesium chloride, potassium sulfate, sodium sulfate, lithium sulfate, potassium acid phosphate, mannitol, urea, inositol, magnesium succinate, tartaric acid, raffinose, sucrose, glucose, lactose, sorbitol, inorganic salts, organic salts, carbohydrates, and the like.

Preferred surfactant and structural polymer chemical and commercial/tradenames may be used interchangeably throughout the specification herein. For clarity the following is a listing of said surfactant and structural polymer chemical and corresponding commercial/tradenames.

| Chemical Name | Tradename(s) |
|---|---|
| Poloxamer 188 | PLURONIC ® F68 = LUTROL ® F68 |
| Poloxamer 237 | PLURONIC ® F87 = LUTROL ® F87 |
| Poloxamer 338 | PLURONIC ® F108 = LUTROL ® F108 |
| Poloxamer 407 | PLURONIC ® F127 = LUTROL ® F127 |
| Polyoxyl 40 stearate | MYRJ ® 52 |
| Polyoxyl 50 stearate | MYRJ ® 53 |
| Polyethylene oxide of | POLYOX ® N10 |
| 100,000 molecular weight | |
| Polyethylene oxide of | POLYOX ® N80 |
| 200,000 molecular weight | |
| Polyethylene oxide of | POLYOX ® N 750 |
| 300,000 molecular weight | |
| Polyethylene oxide of | POLYOX ® N 12K |
| 1,000,000 molecular weight | |
| Polyethylene oxide of | POLYOX ® N 60K |
| 2,000,000 molecular weight | |
| Polyethylene oxide of | POLYOX ® 303 |
| 7,000,000 molecular weight | |

As used herein, unless otherwise noted, the term “hydrophilic polymer” shall mean any polymer which has an affinity for water. The hydrophilic polymer may be water-soluble or water-insoluble.

Suitable examples of water soluble hydrophilic polymers include, but are not limited to, polyvinyl pyrrolidone, polyethylene oxide, polyethylene glycol, polyvinyl alcohol, polyvinyl alcohol-polyethylene glycol copolymer, vinyl acetate-vinyl pyrrolidone copolymer, hydroxypropyl cellulose, hydroxypropyl methylcellulose, methylcellulose, hydroxyethyl cellulose, sodium carboxymethylcellulose, calcium carboxymethylcellulose, polyvinyl acetate polyethylene glycol co-polymer, starches, maltodextrins, sugars, sorbitol, mannitol, sucrose, gelatin, casein, natural gums, alginates, and the like. Preferably, the water soluble hydrophilic polymer is methylcellulose.

Suitable examples of water insoluble hydropillic polymers include, but are not limited to, polyether, a polyester, cellulose acetate, cellulose acetate butyrate, and a polyacrylate. Another embodiment is a drug formulation, in which the granule coated is additionally comprised of a compound selected from the group consisting of polyethylene glycol, polyoxyethylene-polyoxypropylene copolymer, hydroxypropyl cellulose, glycerin, a citric acid ester, dibutyl sebacate, and the like.

Preferred hydrophilic polymer include, polyethers, such as methylcellulose or ethyl cellulose; polyesters, such as polyvinyl acetate; polyacrylates, such as ethylacrylate-methylmethacrylate copolymer; citric acid esters, such as triethyl citrate or acetyl triethylcitrate; polyethylene oxide and polyvinyl pyrrolidone (PVP).

Wherein the present invention, the granule coating is comprised of a multiplicity of layers (i.e. two or more layers), the composition of each layer is independently selected and may be the same or different.

Preferably, the granule coating is a single layer. More preferably, the granule coating is a single layer of methylcellulose.

### Drug Formulations

The OROS® Push -Pull™ system provides high concentrations of surfactant to enhance dissolution of low solubility drug within the system and provide the desirable delivery pattern. When a drug tablet formulated with a high loading of a low solubility drug imbibes water, the membrane of this system splits open as the push layer presses against the large mass of the drug layer. Incorporating a surfactant within the drug layer dissolves a sufficient fraction of the drug such that the drug layer can be smoothly extruded through a relatively small delivery port.

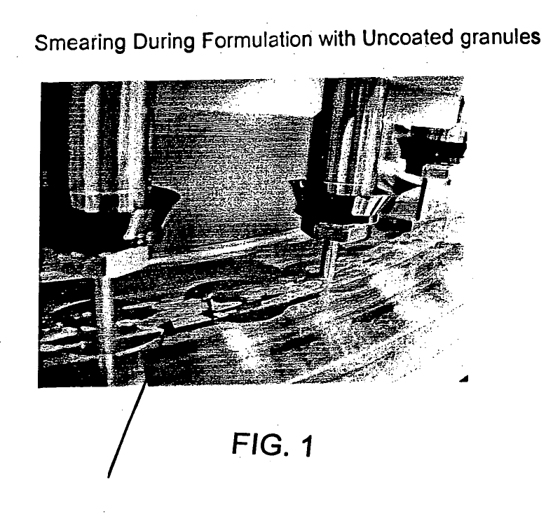

While delivery systems comprising high concentrations of solubilizng agent, preferably surfactant, function very well and can easily be fabricated by hand compression, these systems are difficult to manufacture in high-speed power compression. This is because the drug layer granulations containing the high concentrations of solubilizng agents, preferably, surfactants, are pasty and tend to smear on the tablet press. During high-speed tableting, the smeared material can flake off and result in a loss of control over tablet layer weight and of tablet layer composition. FIG. 1 illustrates the smearing problem of a typical formulation comprising a drug and a solubilizing agent, preferably surfactant, during power compression. The phenomenon observed during the compression is essentially described as smearing of granulated material on the turret of the tablet press. Frictional forces occurring during compression produces continuous build up of smeared material on the turret resulting in stopping of the operation in less than 15 minutes of run time.

Smearing affects the relative weight of the tablet core composition, and an accompanying loss of ability to control the composition and structure of the compression product.

This invention addresses the issues related to compression of drug compositions comprising pharmaceutical agent, preferably topiramate and a solubilibizing agent, preferably surfactant. The specificity of drug/surfactant combination used in the drug granulation requires granule protection in order to eliminate smearing during compression. The approach described in this invention is based on encapsulation of granules by a polymer film. The present invention provides a means to produce drug/surfactant compositions that can be formulated into tablets under power compression without smearing.

The surface of a tablet press during power compression of a topiramate/LUTROL F127 composition was clean with no smearing. This formulation was in the form of coated granules. The coated granules compressed well at high speed with minimal or no smearing.

The film properties that are a key to this invention are plasticity and strength. The present invention provides a means to produce drug/solubilizing agent, preferably, drug/surfactant, compositions that can be formulated into tablets under power compression without smearing.

One aspect of the present invention is a drug formulation comprising a tablet core made by mechanical compression and comprising a solubilizing agent, preferably surfactant, and a low solubility therapeutic drug, preferably topiramate, in which the solubilizng agent (preferably surfactant) or drug or both are in coated granules.

Dosage forms in accord with the present invention are manufactured by standard techniques. For example, the dosage form may be manufactured by a wet granulation technique. In the wet granulation technique, the drug and solubilizing agent, preferably, surfactant, are blended using an organic solvent, such as denatured anhydrous ethanol, as the granulation fluid. The remaining ingredients can be dissolved in a portion of the granulation fluid, such as the solvent described above, and this latter prepared solution is slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven. The dried granules are then sized. Next, magnesium stearate, or another suitable lubricant, is added to the drug granulation, and the granulation is put into milling jars and mixed on a jar mill for up to 10 minutes. The composition is pressed into a layer, for example, in a Manesty® press or a Korsch LCT press. For a bilayered core, the drug-containing layer is pressed and a similarly prepared wet blend of the push layer composition, if included, is pressed against the drug-containing layer. The intermediate compression typically takes place under a force of about 50-100 newtons. Final stage compression typically takes place at a force of 3500 newtons or greater, often 3500-5000 newtons. The single or bilayer compressed cores are fed to a dry coater press, e.g., Kilian® Dry Coater press, and subsequently coated with the wall materials as described above. A like procedure is employed for those cores that are manufactured with a push layer and more than one drug layer, typically on a Korsch multi-layer press.

The present invention also encompasses a drug formulation comprising a capsule core comprised of a semipermeable membrane filled with a mixture comprising a solubilizing agent, preferably a surfactant and a low solubility therapeutic drug, in which the solubiliaing agent (preferably surfactant) or drug or both are within coated granules.

The coated granules may contain solubiliaing agent (preferably surfactant) alone, drug alone, or a mixture of drug and solubilizing agent (preferably surfactant). The coated granules may contain other components. These may include a viscosity agent, a binding agent or an osmotic agent.

The granule coating may be continuous or discontinuous. The coating may be water-soluble. For example the granule coating may be comprised of a compound selected from the group consisting of polyvinyl pyrrolidone, polyethylene oxide, polyethylene glycol, polyvinyl alcohol, polyvinyl alcohol-polyethylene glycol copolymer, vinyl acetate-vinyl pyrrolidone copolymer, hydroxypropyl cellulose, hydroxypropyl methylcellulose, methylcellulose, hydroxyethyl cellulose, sodium carboxymethylcellulose, calcium carboxymethylcellulose, polyvinyl acetate polyethylene glycol co-polymer, a starch, maltodextrin, a sugar, sorbitol, mannitol, sucrose, gelatin, casein, a natural gum, an alginate, and mixtures thereof. Alternatively, the granule coating may be water insoluble. For example, the granule coating may be comprised of a compound selected from the group consisting of a polyether (e.g., ethyl cellulose), a polyester (e.g., polyvinyl acetate), cellulose acetate, cellulose acetate butyrate, a polyacrylate (e.g., ethylacrylate-methylmethacrylate copolymer), and mixtures thereof. Additionally the granule coating may be comprised of a polyethylene glycol, polyoxyethylene-polyoxypropylene copolymer, hydroxypropyl cellulose, glycerin, a citric acid ester (e.g., triethyl citrate or acetyl triethylcitrate or dibutyl sebacate) or mixtures thereof.

The granule coating may be a single layer or a multiplicity of layers.

If a single-layer, the coating is preferably comprised of polyvinyl pyrrolidone or polyethylene oxide. The single-layer coating may be between about 1% and about 3% of the granule weight or about 24% of the granule weight, according to the specific properties chosen for the dosage form.

If the coating is a multiplicity of layers, preferably the coating consists of two layers. For example, the coating may preferably be comprised of an outer coating layer of methylcellulose and an inner coating layer of polyethylene oxide or polyvinyl pyrrolidone.

As will be described in the following paragraphs, the drug formulation may be included as a component in a dosage form, preferably a topiramate dosage form, in which the coated granules contain therapeutic drug, preferably, topiramate.

When drug is present in high dosage amounts, greater than 20% of the drug layer by weight, the present invention may provide a beneficial increased solubility of the lowly soluble drug to provide for creation of a deliverable drug layer. Additionally, the present invention may provide a potentially beneficial increased bioavailability of the lowly soluble drug by increasing its solubility and wetted surface for greater bioadhesion to the gastrointestinal tract mucosa. The wetting properties of solubilizing agents, preferably surfactants, can also have the effect of preventing the released drug and hydrogel carrier from agglomerating, thereby leading to a more complete spreading of the dispensed drug composition onto the absorbable surfaces of the gastrointestinal tract which increased surface area provides more absorption surface area to increase the rate and extent of drug absorbed and increase the therapeutic response. Moreover, the solubilizing agent, preferably surfactant may impart adhesive character to the dispensed drug/hydrogel which adhesive character can prolong in time the contact that the drug/hydrogel makes with the absorbable mucosal tissue of the gastrointestinal tract giving more time for the drug to be spread onto and absorbed once delivered. In yet another potential beneficial effect, the solubilizing agent, preferably surfactant, may additionally increase the permeability of mucosal membranes to the drug molecule which permeability enhancement can lead also to enhanced bioavailability of the drug and enhanced therapeutic response.

When drug of the present invention is present in low dosage amounts, less than 20% of drug layer, the present invention may provide a beneficial increased bioavailability of the lowly soluble drug by increasing its solubility and wetted surface for greater bioadhesion to the gastrointestinal tract mucosa and enhanced permeability of the mucosal surfaces. The increased drug solubility, the increased surface contact area on the mucosal tissue, the increased contact time to the mucosal tissue, and permeability enhancement of the mucosal tissue to the drug molecule may individually or compositely contribute to the overall therapeutic enhancement of the drug by the present invention.

Drug is exemplified herein through the use of topiramate, which is lowly soluble and therapeutically required to be delivered in high doses. Topiramate is in the therapeutic category of anti-convulsants although the drug may be therapeutic for other indications as well.

Solubility of neat topiramate was measured in de-ionized water at 37 degrees centigrade to be 13 mg/ml.

### Topiramate Dosage Form

Another present invention is a dosage form for oral administration of topiramate, comprising a tablet core made by mechanical compression, in which the core comprises a solubilizing agent, preferably a surfactant, and topiramate, and in which the solubilizing agent (preferably surfactant) or topiramate or both are in coated granules.

An aspect of the invention is a topiramate dosage form, further comprising an osmotic delivery system (e.g. a push layer) and a tablet core, in which the osmotic delivery system and the tablet core are surrounded by a semipermeable membrane having a delivery port through the membrane.

Alternatively the tablet core may be incorporated into a matrix delivery system. The matrix system may be either an erodible matrix delivery system or a non-erodible matrix delivery system.

If the present invention is used in an erodible matrix application, the erodible matrix form further comprises a structural polymer, wherein the molecular weight of the structural polymer is selected to modify the erosion rate of the system. High molecular weight polymers are used to produce slow erosion rate and slow delivery of drug, low molecular weight polymers produce faster erosion rate and faster release of drug. A blend of high and low molecular weight structural polymers produces an intermediate delivery rate.

If the present invention is used in a nonerodible porous matrix, the non-eroodible matrix form further comprises a structural polymer wherein the molecular weight of the structural polymer is selected to provide a hydrogel with viscosity within the pores of the matrix. This viscosity suspends drug particles to promote partial or complete dissolution of the drug in the presence of the solubilizing agent, preferably surfactant, prior to delivery from the pores of the dosage

The present invention is further directed to a topiramate dosage form, which is a controlled release topiramate dosage form.

In an embodiment, the controlled release topiramate dosage form comprises a push layer (one type of osmotic delivery system), and a semipermeable wall that is positioned over at least a portion of the push layer.

Sustained release or controlled release dosage forms may be prepared as osmotic dosage forms. Osmotic dosage forms utilize osmotic pressure to generate a driving force for imbibing fluid into a compartment formed, at least in part, by a semi-permeable wall that permits free diffusion of water but not drug or other components. A significant advantage to osmotic systems is that operation is pH-independent and thus continues at the osmotically determined rate throughout an extended time period, even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release 35 (1995) 1-21, incorporated in its entirety by reference herein. In particular, the following U.S. Patents, owned by the assignee of the present application, ALZA Corporation, directed to osmotic dosage forms: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850. Such osmotic dosage forms generally comprise a drug layer, an optional push layer, a semi-permeable membrane which encompasses the drug and push layers and one or more exit orifices.

In the aqueous environment of the gastrointestinal (GI) tract, water is imbibed through the semi-permeable membrane of the osmotic dosage form, at a controlled rate. This causes the push layer to swell and the drug composition(s) to hydrate and form viscous, but deformable, masses. The push layer expands against the drug composition(s), which are pushed out through the orifice. The drug composition(s) exit the system through the exit orifice in the membrane over prolonged periods of time as water from the gastrointestinal tract is imbibed into the delivery system. At the completion of drug release, the biologically inert components of the dosage form are eliminated as a tablet shell.

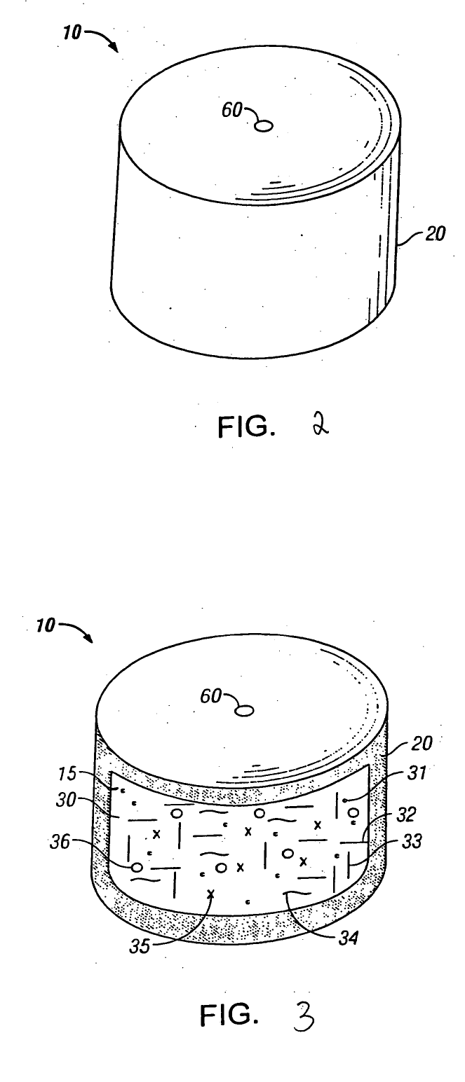

FIG. 2 is a perspective view of one embodiment of a sustained release osmotic dosage form in a standard biconvex round shaped tablet. Dosage form 10 comprises a semi-permeable wall 20 that surrounds and encloses an internal compartment (not seen in FIG. 2). The internal compartment comprises a drug composition comprising a pharmaceutical agent and a solubilizing agent. Semi-permeable wall 20 is provided with at least one exit orifice 60 for connecting the internal compartment with the exterior environment of use. Accordingly, following oral ingestion of dosage form 10, water is imbibed through semi-permeable wall 20 and the pharmaceutical agent/drug composition is released through exit 60.

While the geometrical embodiment in FIG. 2 illustrates a standard biconvex round shaped tablet, the dosage forms of the present invention may embrace other geometries including, a capsule shaped caplet, oval, triangular and other shapes designed for oral administration, including buccal or sublingual dosage forms.

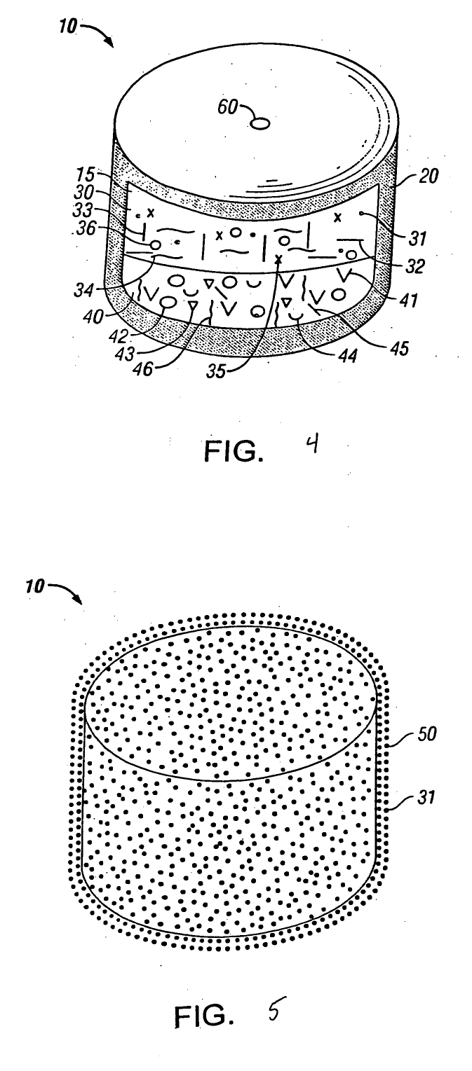

FIG. 3 is a cutaway view of FIG. 2 showing internal compartment 15 containing a single drug composition 30, wherein the drug composition comprises pharmaceutical agent 31 in an admixture with selected excipients. The excipients may be selected to increase the solubility of the drug composition 30 and/or to provide an osmotic activity gradient for driving fluid from an external environment through semi-permeable wall 20 for forming a deliverable drug composition upon imbibition of fluid and/or for other performance and/or manufacturing purposes.

In an embodiment, the present invention is directed to a drug composition 30, wherein the drug composition comprises at least one pharmaceutical agent 31, preferably one to two pharmaceutical agents, more preferably one pharmaceutical agent and a solubilizing agent 33. Preferably the pharmaceutical agent 31 is topiramate. Preferably, the solubilizing agent 33 is a surfactant.

Preferably, drug composition 30 comprises a pharmaceutical agent 31 and a solubilizing agent 33, wherein the pharmaceutical agent 31 is a low solubility and/or a low dissolution rate pharmaceutical agent. Preferably, the drug composition of the present invention comprisies at least about 5%, more preferably, at least about 11%, more preferably, at least about 17.5%, more preferably, at least about 25%, more preferably, at least about 30%, more preferably, at least about 40%, more preferably, at least about 42%, more preferably, at least about 45%, solubilizing agent 33, by weight of the drug composition.

In another embodiment of the present invention, as shown in FIG. 3, the drug composition comprises a pharmaceutical agent 31, a solubilizing agent 33 (represented by vertical dashes) and a structural polymer 32 (represented by horizontal dashed lines).

Drug composition 30 excipients may further optionally include a lubricant 34 (represented by horizontal wavy lines), an osmotically active agent, also known as an osmoagent 35 (represented by “X” symbols) and/or a suitable binder 36 (represented by large circles).

In operation, following oral ingestion of dosage form 10, the osmotic activity gradient across the smei-permeable wall 20 causes water of the gastrointestinal tract to be imbibed through the semi-permeable wall 20, thereby forming a deliverable drug composition, e.g., a solution or suspension or hydrogel, within the internal compartment. The deliverable drug composition is then released through the exit orifice 60 as water continues to enter the internal compartment. As release of the drug composition occurs, water continues to be imbibed thereby driving continued release. In this manner, drug is released in a sustained and continuous manner over an extended time period.

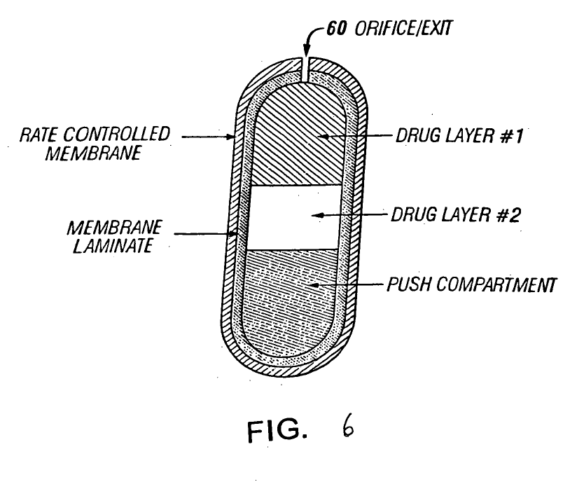

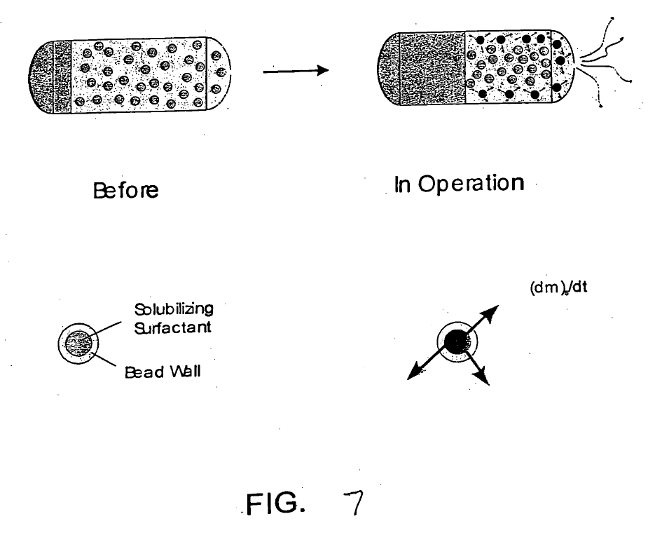

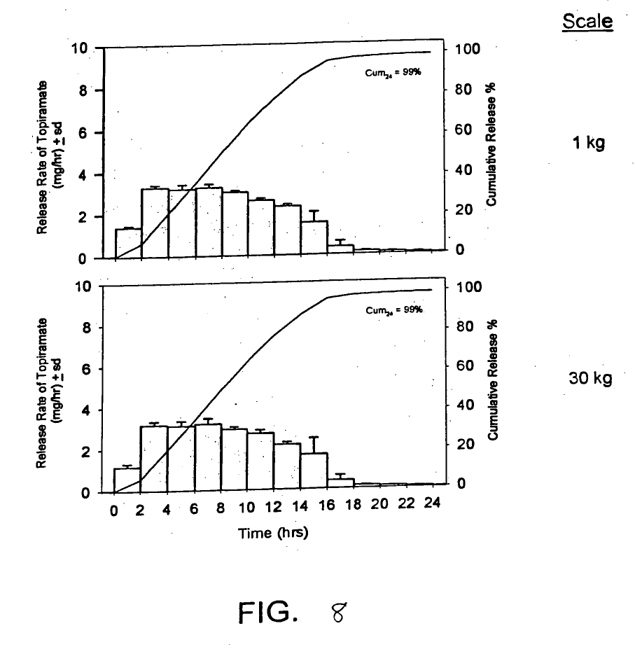

FIG. 4 is a cutaway view of FIG. 2 with an alternate embodiment of internal compartment 15, wherein the internal compartment comprises a bi-layer configuration. In this embodiment, internal compartment 15 contains a bi-layered compressed core having a first drug composition 30 and a push layer 40. Drug composition 30, as described above with reference to FIG. 2 and 3, comprises a pharmaceutical agent and a solubilizing agent, in an admixture with further, optional excipients.

As is described in more detail below, the second component, push layer 40, comprises osmotically active component(s), but does not contain any pharmaceutical agent. In an embodiment of the present invention, push layer 40 comprises osmopolymer 41. Preferably, the components in push layer 40 comprise an osmoagent 42 (represented by very large circles) and one or more osmopolymers 41 (represented by “V” symbols).

Additional, optional excipients within push layer 40, may include binder 43 (represented by down-ward triangles), lubricant 44 (represented by upward semi-circles), antioxidant 45 (represented by diagonal lines) and/or colorant 46 (represented by vertical wavy lines).

As water is imbibed through the semi-permeable wall 20, the osmopolymer(s) within push layer 40 swell and push against drug composition 30 to thereby facilitate release of the drug composition through the exit orifice 60 and thus the pharmaceutical agent from the dosage form.

In an embodiment of the present invention, drug composition 30, as described with reference to FIGS. 3 and 4 comprises a pharmaceutical agent (for example, topiramate) and solubilizing agent 33 in an admixture with further, optional, selected excipients. The excipients may be one or more selected from a structural polymer 32, lubricant 34, an osmoagent 35 and/or a binder 36.

In another embodiment of the present invention, push layer 40, as described with reference to FIG. 4, comprises osmotically active components, more specifically an osmoagent 42 and an osmopolymer 41, but does not contain any pharmaceutical agent.

FIG. 5 is a view of another embodiment of the present invention, a biconvex round standard tablet as in FIG. 2, wherein the tablet includes a further, optional immediate release coating 50 of a pharmaceutical agent, preferably topiramate, covering the dosage form of FIG. 2, 3 or 4.

More specifically, dosage form 10 of FIG. 5 comprises an overcoat 50 on the outer surface of semi-permeable wall 20 of dosage form 10. Overcoat 50 is a drug composition comprising about 10 μg to about 500 mg of drug 31, preferably, overcoat 50 comprises about 10 μg to about 200 mg of drug 31, more preferably, overcoat 50 comprises about 5 mg to about 100 mg of drug 31 and from about 5 mg to about 200 mg of a pharmaceutically acceptable carrier selected from the group consisting of alkylcellulose, hydroxyalkylcellulose and hydroxypropylalkylcellulose. The overcoat pharmaceutically acceptable carrier is represented by a polymer or copolymer such as methylcellulose, hydroxyethylcellulose, hydroxybutylcellulose, hydroxypropylcellulose, hydroxypropylmethylcellulose, hydroxypropylethylcellulose and hydroxypropylbutylcellulose, polyvinyl pyrrolidone/vinyl acetate copolymer, polyvinyl alcohol-polyethylene graft copolymer, and the like. Overcoat 50 provides immediate release of the pharmaceutical agent, as overcoat 50 dissolves in the presence of gastrointestinal fluid and concurrently therewith delivers drug 31 into the gastrointestinal tract for immediate therapy. Drug 31 in overcoat 50 can be the same or different than the drug 31 in drug composition 30. Preferably drug 31 in overcoat 50 is the same as drug 31 in drug composition 30. More preferably drug 31 is topiramate.

FIG. 6 illustrates another, preferred embodiment of the present invention, illustrating an open view of a tri-layer capsule shaped osmotic dosage form. FIG. 6 illustrates a capsule shaped tablet embodiment of the present invention comprising a first drug composition 30, a second drug composition 70 and a push layer 40. The capsule shaped core (comprising the first and second drug compositions and the push layer) is enveloped by semi-permeable membrane 20. The dosage form further comprises at least one exit orifice 60 which exposes the first drug composition 30 to the environment of use. The dosage form in FIG. 6 further comprises an additional, optional inner membrane 80 that may function as a flow-promoting layer and/or as a smoothing layer and/or contribute to the control of the rate of imbibition of water into the dosage form.

In an embodiment of the present invention, as described in FIG. 6, the amount and/or concentration of the drug in the first drug composition 30 is different than the amount and/or concentration of drug in second drug composition 70. In another embodiment of the present invention, the amount and/or concentration of drug in the first drug composition 30 is less than the amount and/or concentration of drug in second drug composition 70. Preferably, the amount and/or concentration of drug in the first drug composition 30 is less than the amount and/or concentration of drug in the second drug composition 70. More preferably, the amounts and/or concentrations of drug in the first and second drug compositions are selected to yield a substantially ascending rate of release of the pharmaceutical agent.

The dosage form illustrated in FIG. 6 may further comprise additional drug compositions having varying drug amounts and/or concentrations, to provide alternate release rates and/or patterns and/or to achieve alternate drug plasma concentration profiles that may be preferred.

The drug composition of the present invention comprises two components: (a) a pharmaceutical agent 31, preferably a low solubility and/or low dissolution rate pharmaceutical agent, more preferably topiramate, and (b) a solubilizing agent 33, preferably a surfactant. In an embodiment of the present invention, the drug composition comprises (a) a pharmaceutical agent 31, preferably a low solubility and/or low dissolution rate pharmaceutical agent, more preferably, topiramate, (b) a solubilizing agent 33, preferably a surfactant and (c) a structural polymer 32. The drug composition may further, optionally contain one or more excipients, as herein described.

In a preferred embodiment of the present invention, the pharmaceutical agent in drug layer 30 is present in a therapeutically effective amount. In another embodiment of the present invention, the total amount of pharmaceutical agent present in the drug compostion or compositions of the dosage forms of the present invention, is equal to or greater than the therapeutically effective, recommended or desired daily dosage.

In an embodiment of the present invention, the pharmaceutical agent in drug composition 30 (or wherein the dosage form comprises more than one drug composition, the pharmaceutical agent in the combined drug compositions) is present in an amount equal to or greater than the recommended or desired daily dosage of the pharmaceutical agent to be administered to a patient in need thereof, thereby permitting once-a-day or less frequent dosing.

Wherein the dosage form contains more than one drug composition, as for example in FIG. 6 wherein two drug compositions 30 and 70 are present, each drug composition comprises independently selected (a) pharmaceutical agent 31, preferably a low solubility and/or low dissolution rate pharmaceutical agent, more preferably topiramate and (b) solubilizing agent 33, preferably surfactant. Each drug composition may further optionally contain independently selected structural polymer 32 and/or one or more independently selected excipients as hereinafter described.

Wherein two or more drug compositions are present within the dosage forms of the present invention, the daily dosage of the pharmaceutical agent is present in divided amounts. For example, if the dosage of the pharmaceutical agent is 400 mg, and the dosage form comprises two drug compositions (e.g. drug compositions 30 and 70 as exemplified in FIG. 6), then the sum of the amount of pharmaceutical agent in the first drug composition plus the amount of pharmaceutical agent in the second drug composition will total 400 mg or more.

Wherein two drug compositions are present with the dosage forms of the present invention, the ratio of the drug concentration in the second drug composition 70 to the drug concentration in the first drug composition 30, as illustrated in FIG. 6, is preferably in the range of from about 1.0 to about 2.5, preferably, about 1.0 to about 2.0, more preferably, about 1.25 and about 1.75.

Pharmaceutical agent 31 is preferably a low solubility and/or low dissolution rate pharmaceutical agent, more preferably, topiramate. Topiramate is in the therapeutic category of anticonvulsants. The solubility of neat topiramate is in the range of about 9.8 mg/ml to 13.0 mg/ml, with solubility in de-ionized water measured to be about 12 mg/ml.

Pharmaceutical agent 31 may be provided in the drug composition in an amount in the range of from about 1 mg to about 750 mg per dosage form. Preferably, the pharmaceutical agent is present in an amount in the range of from about 1 mg to about 250 mg per dosage form, and more preferably, in the range of from about 5 mg to about 250 mg. The amount of pharmaceutical agent within the dosage form will depend upon the required dosing level that must be maintained over the delivery period, i.e., the time between consecutive administrations of the dosage forms. In an embodiment of the present invention, the pharmaceutical agent is present in an amount in the range of from about 5 mg to about 250 mg, more preferably, in an amount in the range of from about 10 mg to about 250 mg per day.

Preferably, pharmaceutical agent 31 is present in the drug composition in micronized form. Preferably, the micronized pharmaceutical agent has a nominal particle size of less than about 200 microns, more preferably less than about 100 microns, most preferably, less than about 50 microns.

Solubilizing agent 33, preferably a pharmaceutically acceptable solubilizing agent, more preferably, a surfactant, is included in the drug composition(s) of the dosage forms of the present invention, as represented by vertical dashes in FIG. 3 and FIG. 4.

It is well known that solubilizing agents, more particularly surfactants, can be used in liquid drug delivery systems as wetting agents, drug solubilizers, meltable carriers, oily liquid fills in gel capsules for oral administration, parenteral liquids for injection, ophthalmic drops, topical ointments, salves, lotions, and creams, suppositiories, and in pulmonary and nasal sprays. By their amphipathic molecular structure comprising opposing polar hydrophilic and non-polar hydrophobic moieties with opposite physical and chemical properties, surfactants are well known to have poor cohesive properties. Accordingly, surfactants have been limited to the above applications because at room temperature, such surfactants are in the physical form of liquids, pastes, or brittle solids, which physical forms and properties are generally unacceptable for use as components in compressed solid tablets sufficiently durable for manufacture and practical use.

As noted, surfactants typically have poor cohesive properties and therefore do not compress as hard, durable tablets. Furthermore, surfactants are in the physical form of liquid, pastes, or waxy solids at standard temperatures and conditions and are inappropriate for tabletted oral pharmaceutical dosage forms. However, it has been unexpectedly found that surfactants may be used in accordance with the drug compositions and dosage forms of the present invention to enhance the solubility of the pharmaceutical agent and potentially, the bioavailability of the pharmaceutical agent.

A class of solubilizing agents which may be used in the drug compositions and/or dosage forms of the present invention include, for example, a surfactant of POLYOXYL 40 stearate (also known as MYRJ 52) and POLYOXYL 50 stearate (also known as MYRJ 53). Preferably, the solubilizing agent is a drug solubilizing surfactant selected from the group polyethylene glycol (PEG) 3350; PEG 8K; KOLLIDON K90; LUROL F 68, F87, F127, F108; MYRJ 52S; and PVP K2939. Preferably, the solubilizing agent is the surfactant LUTROL F127.

Another class of surfactant which may be used in the drug compositions and/or dosage forms of the present invention is a group of co-polymers of ethylene oxide and propylene oxide conforming to the general formula OH(C₂H₄O)ₐ(C₃H₆O)_{b}(C₂H₄O), also known as poloxamers or by their tradenames PLURONICs and LUTROLs. In this class of surfactants, the hydrophilic ethylene oxide ends of the surfactant molecule and the hydrophobic midblock of propylene oxide of the surfactant molecule serve to dissolve and suspend the drug in the pumpable hydrogel.

Other surfactants that are solids at room temperature and which may be used in the drug compositions and/or dosage forms of the present invention include members selected from the group essentially consisting of sorbitan monopalmitate, sorbitan monostearate, glycerol monostearate, polyoxyethlene stearate (self emulsifying), polyoxyethylene 40 sorbitol lanolin derivative, polyoxyethylene 75 sorbitol lanolin derivative, polyoxyethylene 6 sorbitol beeswax derivative, polyoxyethylene 20 sorbitol beeswax derivative, polyoxyethylene 20 sorbitol lanolin derivative, polyoxyethylene 50 sorbitol lanolin derivative, polyoxyethylene 23 lauryl ether, polyoxyethylene 23 lauryl ether with butylated hydroxyanisole and citric acid added as preservatives, polyoxyethylene 2 cetyl ether with butylated hydroxyanisole and citric acid added as preservatives, polyoxyethylene 10 cetyl ether with butylated hydroxyanisole and citric acid added as preservatives, polyoxyethylene 20 cetyl ether with butylated hydroxyanisole and citric acid added as preservatives, polyoxyethylene 2 stearyl ether with butylated hydroxyanisole and citric acid added as preservatives, polyoxyethylene 10 stearyl ether with butylated hydroxyanisole and citric acid added as preservatives, polyoxyethylene 20 stearyl ether with butylated hydroxyanisole and citric acid added as preservatives, polyoxyethylene 21 stearyl ether with butylated hydroxyanisole and citric acid added as preservatives, polyoxyethylene 20 oleyl ether with butylated hydroxyanisole and citric acid added as preservatives, polyoxyethylene 40 stearate, polyoxyethylene 50 stearate, polyoxyethylene 100 stearate, sorbitan monopalmitate, sorbitan monostearate, sorbitan tristearate, polyoxyethylene 4 sorbitan monostearate, polyoxyethylene 20 sorbitan tristearate, and the like. “Handbook of Pharmaceutical Excipients”, 2^{nd} Ed. Ainley Wade and Paul J. Weller Editors, 1994.

An especially preferred family of surfactants are a:b:a triblock co-polymers of ethylene oxide:propylene oxide:ethylene oxide. The “a” and “b” represent the average number of monomer units for each block of the polymer chain. These surfactants are commercially available from BASF Corporation of Mount Olive, N.J., in a variety of different molecular weights and with different values of “a” and “b” blocks. For example, LUTROL® F127 has a molecular weight range of 9,840 to 14,600 and where “a” is approximately 101 and “b” is approximately 56, LUTROL F87 represents a molecular weight of 6,840 to 8,830 where “a” is 64 and “b” is 37, LUTROL F108 represents an average molecular weight of 12,700 to 17,400 where “a” is 141 and “b” is 44, and LUTROL F68 represents an average molecular weight of 7,680 to 9,510 where “a” has a value of about 80 and “b” has a value of about 27. A resource of surfactants including solid surfactants and their properties is available in McCutcheon's Detergents and Emulsifiers, International Edition 1979 and McCutcheon's Detergents and Emulsifiers, North American Edition 1979. Other sources of information on properties of solid surfactants include BASF Technical Bulletin PLURONIC & TETRONIC Surfactants 1999 and General Characteristics of Surfactants from ICI Americas Bulletin 0-1 10/80 5M.

One of the characteristics of surfactants tabulated in these references is the HLB value, or hydrophilic lipophilic balance value. This value represents the relative hydroplicility and relative hydrophobicity of a surfactant molecule. Generally, the higher the HLB value, the greater the hydrophilicity of the surfactant while the lower the HLB value, the greater the hydrophobicity. For the LUTROL molecules, for example, the ethylene oxide fraction represents the hydrophilic moiety and the propylene oxide fraction represents the hydrophobic fraction. The HLB values of LUTROL F127, F87, F108, and F68 are respectively 22.0, 24.0, 27.0, and 29.0.

Other particularly preferred surfactants include sugar ester surfactants, which are sugar esters of fatty acids. Such sugar ester surfactants include sugar fatty acid monoesters, sugar fatty acid diesters, triesters, tetraesters, or mixtures thereof, although mono- and di-esters are most preferred. Preferably, the sugar fatty acid monoester comprises a fatty acid having from 6 to 24 carbon atoms, which may be linear or branched, or saturated or unsaturated C₆ to C₂₄ fatty acids. The C₆ to C₂₄ fatty acids include C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, and C₂₄ in any subrange or combination. These esters are preferably chosen from stearates, behenates, cocoates, arachidonates, palmitates, myristates, laurates, carprates, oleates, laurates and their mixtures.

Preferably, the sugar fatty acid monoester comprises at least one saccharide unit, such as sucrose, maltose, glucose, fructose, mannose, galactose, arabinose, xylose, lactose, sorbitol, trehalose or methylglucose. Disaccharide esters such as sucrose esters are most preferable, and include sucrose cocoate, sucrose monooctanoate, sucrose monodecanoate, sucrose mono- or dilaurate, sucrose monomyristate, sucrose mono- or dipalmitate, sucrose mono- and distearate, sucrose mono-, di- or trioleate, sucrose mono- or dilinoleate, sucrose polyesters, such as sucrose pentaoleate, hexaoleate, heptaoleate or octooleate, and mixed esters, such as sucrose palmitate/stearate.

Particularly preferred examples of such sugar ester surfactants include those sold by the company Croda Inc of Parsippany, N.J. under the names CRODESTA F10, F50, F160, and F110 denoting various mono-, di- and mono/di ester mixtures comprising sucrose stearates, manufactured using a method that controls the degree of esterification, such as described in U.S. Pat. No. 3,480,616. These preferred sugar ester surfactants provide the added benefit of tabletting ease and nonsmearing granulation. The sugar ester surfactants may also provide enhanced compatibility with sugar based therapeutic agents, exemplified by topiramate.

Sugar surfactants sold by the company Mitsubishi under the name RYOTO SUGAR ESTERS, for example under the reference B370 corresponding to sucrose behenate formed of 20% monoester and 80% di-, tri- and polyester may also be used. Use may also be made of the sucrose mono- and dipalmitate/stearate sold by the company Goldschmidt under the name “TEGOSOFT PSE”. Use may also be made of a mixture of these various products. The sugar ester can also be present in admixture with another compound not derived from sugar; and a preferred example includes the mixture of sorbitan stearate and of sucrose cocoate sold under the name “ARLATONE 2121” by the company ICI. Other sugar esters include, for example, glucose trioleate, galactose di-, tri-, tetra- or pentaoleate, arabinose di-, tri- or tetralinoleate or xylose di-, tri- or tetralinoleate, or mixtures thereof. Other sugar esters of fatty acids include esters of methylglucose include the distearate of methylglucose and of polyglycerol-3 sold by the company Goldschmidt under the name of TEGOCARE 450. Glucose or maltose monoesters can also be included, such as methyl O-hexadecanoyl-6-D-glucoside and O-hexadecanoyl-6-D-maltose. Certain other sugar ester surfactants include oxyethylenated esters of fatty acid and of sugar include oxyethylenated derivatives such as PEG-20 methylglucose sesquistearate, sold under the name “GLUCAMATE SSE20”, by the company Amerchol.

Solubilizing agent 33 can be one surfactant or a blend of surfactants. The surfactants are selected such that they have values that promote the dissolution and solubility of the drug. A high HLB surfactant can be blended with a surfactant of low HLB to achieve a net HLB value that is between them, if a particular drug requires the intermediate HLB value. Surfactant 33 is selected depending upon the drug being delivered; such that the appropriate HLB grade is utilized.

Preferably, the solubilizing agent is selected from the group consisting of MYRJ 52, MYRJ 53, MYRJ 59FL, KOLLIDON 12PF, KOLLIDON 17PF, KOLLIDON 25/30, KOLLIDON K90, LUTROL F68, LUTROL F87, LUTROL F127, LUTROL F108; PVP K2932, polyethylene glycol (PEG) 3350; PEG 8K; sorbitan monopalmitate, sorbitan monostearate, glycerol monostearate, polyoxyethlene stearate, sucrose cocoate, polyoxyethylene 40 sorbitol lanolin derivative, polyoxyethylene 75 sorbitol lanolin derivative, polyoxyethylene 6 sorbitol beeswax derivative, polyoxyethylene 20 sorbitol beeswax derivative, polyoxyethylene 20 sorbitol lanolin derivative, polyoxyethylene 50 sorbitol lanolin derivative, polyoxyethylene 23 lauryl ether, polyoxyethylene 23 lauryl ether with butylated hydroxyanisole and citric acid added as preservatives, polyoxyethylene 2 cetyl ether with butylated hydroxyanisole and citric acid added as preservatives, polyoxyethylene 2 stearyl ether, polyoxyethylene 21 stearyl ether, polyoxyethylene 100 stearyl ether, polyoxyethylene 10 cetyl ether with butylated hydroxyanisole and citric acid added as preservatives, polyoxyethylene 20 cetyl ether with butylated hydroxyanisole and citric acid added as preservatives, polyoxyethylene 2 stearyl ether with butylated hydroxyanisole and citric acid added as preservatives, polyoxyethylene 10 stearyl ether with butylated hydroxyanisole and citric acid added as preservatives, polyoxyethylene 20 stearyl ether with butylated hydroxyanisole and citric acid added as preservatives, polyoxyethylene 21 stearyl ether with butylated hydroxyanisole and citric acid added as preservatives, polyoxyethylene 20 oleyl ether with butylated hydroxyanisole and citric acid added as preservatives, polyoxyethylene 40 stearate, polyoxyethylene 50 stearate, polyoxyethylene 100 stearate, sorbitan monopalmitate, sorbitan monostearate, sorbitan tristearate, polyoxyethylene 4 sorbitan monostearate, polyoxyethylene 20 sorbitan tristearate, and mixtures thereof.

More preferably, the solubilzing agent is a surfactant selected from the group consisting of LUTROL F68, LUTROL F87, LUTROL 108, LUTROL F127, MYRJ 52, MYRJ 53; most preferably, the solubilizing agent is the surfactant LUTROL F127.

In a preferred embodiment of the present invention, the surfactant is not a sugar ester.

Preferably, in the drug compositions and/or dosage forms of the present invention, the pharmaceutical agent is matched with a suitable, aforementioned solubilizing agent, preferably, a solid surfactant or mixture of surfactants.

A suitable surfactant may be selected by preparing aqueous solutions of selected surfactants spanning a range of HLB values and a range of concentrations. Then, the pharmaceutical agent is added in excess to the surfactant solutions and the saturated solubility of the pharmaceutical agent (at equilibrium) is measured by an appropriate analytical method such as ultraviolet spectroscopy, chromatographic methods, or gravimetric analysis. The solubility values are then plotted as a function of HLB and as a function of surfactant concentration. The solubilizing agent (preferably surfactant) can then be selected by evaluating the maximum point of solubility generated in the plots at the different concentrations.

Preferably, wherein the pharmaceutical agent is topiramate, the solubilizing agent is a surfactant, preferably, the surfactant is PLURONIC F127 or its corresponding pharmaceutically acceptable grade LUTROL F127.

Preferably, the solubilizing agent, preferably surfactant, is present in the drug composition in micronized form. Preferably, the micronized solubilizing agent, preferably surfactant, has a nominal particle size of less than about 200 microns, more preferably, less than about 100 microns, most preferably, less than about 50 microns.

To achieve a substantially zero order release rate profile, the ratio of solubilizing agent, preferably surfactant, to pharmaceutical agent is preferably, in the range of from about 1.3 to about 2.7, more preferably, in the range of from about 1.5 to about 2.5, more preferably still, in the range of from about 1.8 to about 2.2.

To achieve a substantially ascending release rate profile, the ratio of solubilizing agent, preferably surfactant, to pharmaceutical agent is preferably, in the range of from about 0.1:1 to about 3:1, more preferably, in the range of from about 0.25:1 to about 2.5:1, more preferably, in the range of from about 0.5:1 to about 2:1, more preferably still, in the range of from about 1:1 to about 2:1, more preferably still, in the range of from about 1.5:1 to about 2:1.

The present invention may provide a potentially beneficial increased bioavailability to the low solubility and/or low dissolution rate drug by increasing its solubility and wetted surface for greater bioadhesion to the gastrointestinal tract mucosa. The wetting properties of the solubilizing agent (preferably surfactant) may also have the effect of preventing the released drug from agglomerating upon release into the environment of use, thereby leading to a more complete spreading of the dispensed drug composition onto the absorbable surfaces of the gastrointestinal tract. The resulting increased surface area may provide more absorption surface area to increase the rate and extent of drug absorbed and thus increase the therapeutic response.

The solubilizing agent (preferably surfactant) may further impart adhesive character to the dispensed drug composition, which adhesive character may prolong the contact time between the drug composition and the absorbable mucosal tissue of the gastrointestinal tract, thereby providing more time for the drug to be spread and be absorbed once delivered.

In yet another potential beneficial effect, the solubilizing agent (preferably surfactant) may additionally increase the permeability of mucosal membranes to the drug molecule which permeability enhancement may lead to enhanced bioavailability of the drug and enhanced therapeutic response.

When drug 31 is present in low dosage amounts, less than about 20% by weight of the drug composition 30, the present invention may provide a beneficial increased bioavailability of the low solubility and/or low dissolution rate drug, by increasing its solubility and wetted surface for greater bioadhesion to the gastrointestinal tract mucosa and enhanced permeability of the mucosal surfaces. The increased drug solubility, the increased surface contact area on the mucosal tissue, the increased contact time to the mucosal tissue, and permeability enhancement of the mucosal tissue to the drug molecule may individually or compositely contribute to the overall therapeutic enhancement of the drug by the present invention.

Structural polymer 32 comprises any component, for example a hydrophilic polymer, which provides cohesiveness to the blend so durable tablets can be made. The structural polymer may also form a hydrogel for viscosity control during the operation of the delivery system. The structural polymer further suspends the drug particles to promote partial or complete solubilization of the drug within the dosage form prior to delivery from the dosage form.

The molecular weight of the structural polymer 32 may be chosen to impart desired properties to the dosage form, and more particularly to the drug compositions within the dosage form. High molecular weight polymers are used to produce a slow hydration rate and slow delivery of drug, whereas low molecular weight polymers produce a faster hydration rate and faster release of drug. A blend of high and low molecular weight structural polymers produces an intermediate delivery rate.

If the drug composition of the present invention is used in an erodible matrix dosage form, the molecular weight of the structural polymer is selected to modify the erosion rate of the system. High molecular weight polymers are used to produce slow erosion rate and slow delivery of drug, whereas low molecular weight polymers produce a faster erosion rate and faster release of drug. A blend of high and low molecular weight structural polymers produces an intermediate delivery rate.

If the drug composition of the present invention is used in a non-erodible porous matrix dosage form, the molecular weight of the structural polymer is selected to provide a viscous hydrogel within the pores of the matrix. The viscosity of the hydrogel serves to suspends drug particles to promote partial or complete solubilization of the drug in the presence of the surfactant prior to delivery from the pores of the dosage form.

Structural polymer 32 is a hydrophilic polymer particle in the drug composition that contributes to the controlled delivery of active agent. Representative examples of suitable structural polymers include, but are not limited to, poly(alkylene oxide) of 100,000 to 750,000 number-average molecular weight, including poly(ethylene oxide), poly(methylene oxide), poly(butylene oxide) and poly(hexylene oxide); and a poly(carboxymethylcellulose) of 40,000 to 1,000,000 400,000 number-average molecular weight, represented by poly(alkali carboxymethylcellulose), poly(sodium carboxymethylcellulose), poly(potassium carboxymethylcellulose) poly(calcium carboxymethylcellulose), and poly(lithium carboxymethylcellulose). The drug composition may alternatively comprise a hydroxypropylalkylcellulose of 9,200 to 125,000 number-average molecular weight for enhancing the delivery properties of the dosage form such as hydroxypropylethylcellulose, hydroxypropylmethylcellulose, hydroxypropylbutylcellulose, hydroxypropylpentylcellulose, and the like; and/or a poly(vinylpyrrolidone) of 7,000 to 75,000 number-average molecular weight for enhancing the flow properties of the dosage form. Preferred structural polymers are the poly(ethylene oxide) polymers of 100,000-300,000 number average molecular weight. Structural polymers that erode in the gastric environment, i.e., bioerodible structural polymers, are especially preferred.

Other structural polymers that may be incorporated into drug composition 30 include carbohydrates that exhibit sufficient osmotic activity to be used alone or with other osmoagents. Such carbohydrates comprise monosaccharides, disaccharides and polysaccharides. Representative examples include, but are not limited to, maltodextrins (i.e., glucose polymers produced by the hydrolysis of grain starch such as rice or corn starch) and the sugars comprising lactose, glucose, raffinose, sucrose, mannitol, sorbitol, zylitol and the like. Preferred maltodextrins are those having a dextrose equivalence (DE) of about 20 or less, preferably maltodextrins with a DE ranging from about 4 to about 20, and more preferably from about 9 to about 20. Maltodextrins having a DE of about 9-12 and molecular weight of about 1,600 to 2,500 are preferred.

The carbohydrates described above, preferably the maltodextrins, may be used in the drug composition 30 without the addition of an osmoagent, to yield the desired release of pharmaceutical agent from the dosage form, while providing a therapeutic effect over a prolonged period of time and up to 24 hours with once-a-day dosing.

Preferably, the structural polymer is selected form the group consisting of poly(ethylene oxide), poly(methylene oxide), poly(butylene oxide) and poly(hexylene oxide); poly(carboxymethylcellulose), poly(alkali carboxymethylcellulose), poly(sodium carboxymethylcellulose), poly(potassium carboxymethylcellulose) poly(calcium carboxymethylcellulose), poly(lithium carboxymethylcellulose), hydroxypropylcellulose, hydroxypropylethylcellulose, hydroxypropylmethylcellulose, hydroxypropylbutylcellulose, hydroxypropylpentylcellulose, poly(vinylpyrrolidone), bioerodible structural polymers (carriers), maltodextrins, polyvinyl pyrrolidones, polyvinylpyrrolidone vinyl acetate copolymers, lactose, glucose, raffinose, sucrose, mannitol, sorbitol, zylitol and mixtures thereof.

More preferably, the structural polymer is selected from the group consisting of MALTRIN M100, POLYOX N10 and POLYOX N80, most preferably, the structural polymer is POLYOX N80.

It has been further found that, when present, the structural polymer and solubilizing agent (preferably surfactant) are preferably present in the drug composition in a certain amounts. Preferably, the structural polymer should be present in an amount less than or equal to about 90% by weight of the drug composition and the surfactant should be present in amount between 0 and about 50% by weight of the drug composition. Preferably, for high dosages, the structural polymer should be present in an amount less than or equal to about 30% by weight of the drug composition, more preferably in an amount less than about 20% by weight of the drug composition; and the surfactant should be present in amount greater than or equal to about 15% by weight of the drug composition, more preferably, in an amount greater than or equal to about 25% by weight of the drug composition, more preferably still, in an amount greater than or equal to about 35% by weight of the drug composition, most preferably, in an amount greater than or equal to about 40% by weight of the drug composition.

For high dosages, a preferred range of concentration of structural polymer within the drug composition of osmotic delivery systems is from about 5% to about 50% weight percent of polyoxyethylene 200,000 molecular weight (POLYOX N80), with an especially preferred range of from 0 to about 20% by weight of the drug composition.

For low dosages, a preferred range of concentration of structural polymer within the drug composition of osmotic delivery systems is from about 50% to about 90% weight percent of polyoxyethylene 200,000 molecular weight (POLYOX N80), with an especially preferred range of from 75% to about 90% by weight of the drug composition.

Lubricant 34 may optionally be included in the drug composition as represented by a horizontal wavy line in FIG. 3 and FIG. 4. Lubricant 34 is used during tablet manufacture to prevent adherence to die walls or punch faces. Typical lubricants include, but are not limited to, magnesium stearate, sodium stearate, stearic acid, calcium stearate, magnesium oleate, oleic acid, potassium oleate, caprylic acid, sodium stearyl fumarate, and magnesium palmitate or blends of such lubricants. The amount of lubricant present in the drug composition is preferably, in the range of from about 0.01 to about 20 mg.

Binder 36, preferably a therapeutically acceptable vinyl polymer binder, may also be optionally included in the drug composition as represented by small circles in FIG. 3 and FIG. 4. Representative binders include, but are not limited to vinyl polymer binder, acacia, starch and gelatin. Wherein the binder is a vinyl polymer, the vinyl polymer comprises a 5,000 to 350,000 average molecular weight, represented by a member selected from the group consisting of poly-n-vinylamide, poly-n-vinylacetamide, poly(vinyl pyrrolidone), also known as poly-n-vinylpyrrolidone, poly-n-vinylcaprolactone, poly-n-vinyl-5-methyl-2-pyrrolidone, and poly-n-vinylpyrrolidone copolymers with a member selected from the group consisting of vinyl acetate, vinyl alcohol, vinyl chloride, vinyl fluoride, vinyl butyrate, vinyl laureate, and vinyl stearate. Representative other binders suitable for formulation in the drug composition include, but are not limited to acacia, starch and gelatin. The binder present within the drug composition is preferably, in an amount in the range of from about 0.01 to about 25 mg.

Disintegrants may also be optionally included in the drug composition. Disintegrants may be selected from starches, clays, celluloses, algins and gums and crosslinked starches, celluloses and polymers. Representative disintegrants include, but are not limited to, corn starch, potato starch, croscarmelose, crospovidone, sodium starch glycolate, VEEGUM HV, methylcellulose, agar, bentonite, carboxymethylcellulose, alginic acid, guar gum, low-substituted hydroxypropyl cellulose, microcrystalline cellulose, and the like.

In an embodiment of the present invention, at least one drug composition within a dosage form comprises a pharmaceutical agent and a solubilizing agent. Preferably, the pharmaceutical agent is topiramate and the solubilizing agent is a surfactant, more preferably, the solubilizing agent is the surfactant PLURONIC F127 or its corresponding pharmaceutically acceptable grade LUTROL F127.

It has further been found that the surfactant appears to be capable of operating as both a structural polymer as well as a surfactant, and as such it may be utilized as the sole excipient in the drug composition.

Wherein a drug composition comprises pharmaceutical agent 31, solubilizing agent 33, preferably a surfactant, and structural polymer 32, the amount of structural polymer 32 and surfactant 33 formulated within said drug composition must be appropriately selected and controlled.

One skilled in the art will recognize that the amounts of solubilizing agent and structural polymer are selected to optimize the characteristics of the drug layer composition. The amounts are selected such that the dosage form maintains structural integrity before administration and upon administration, the drug layer composition hydrates and is capable of being pushed out of the dosage form providing a desired release pattern.

In an embodiment of the present invention is a drug composition, wherein the pharmaceutical agent is topitamate and wherein the topiramate is present in amount in the range of about 10 mg to about 200 mg. In further embodiments of the present invention are drug compositions wherein topiramate is present in 10 mg, 20 mg, 40 mg, 45 mg, 80 mg, 90 mg, 120 mg, 135 mg, 160 mg, 180 mg and 200 mg amount.

In an embodiment of the present invention is a dosage form comprising one or more drug compositions, preferably one to two drug compositions, wherein the total amount of topiramate present within the dosage form (i.e. the total amount present within the drug compositions) is in an amount in the range of about 10 mg to about 200 mg. In further embodiments of the present invention are dosage forms comprising one or two drug compositions wherein the total amount of topiramate present is 10 mg, 20 mg, 40 mg, 45 mg, 80 mg, 90 mg, 120 mg, 135 mg, 160 mg, 180 mg or 200 mg amount.

In an embodiment of the present invention is a dosage form comprising a first drug composition comprising pharmaceutical agent, preferably a low solubility and/or low dissolution rate solubilizing agent, more preferably topiramate and a solubilizing agent, preferably surfactant; and a second drug composition comprising pharmaceutical agent, preferably a low solubility and/or low dissolution rate solubilizing agent, more preferably topiramate and a solubilizing agent, preferably surfactant.

In another embodiment of the present invention is a dosage form comprising (a) a core comprising a first drug composition comprising pharmaceutical agent, preferably a low solubility and/or low dissolution rate solubilizing agent, more preferably topiramate and a solubilizing agent, preferably surfactant; and a push layer comprising an osmopolymer; (b) a semi-permeable wall surrounding said core and (c) an exit orifice through the semi-permeable wall for releasing the pharmaceutical agent from the dosage form over a prolonged period of time.

In yet another embodiment of the present invention is a dosage form comprising (a) a core comprising a first drug composition comprising pharmaceutical agent, preferably a low solubility and/or low dissolution rate solubilizing agent, more preferably topiramate and a solubilizing agent, preferably surfactant; a second drug composition comprising pharmaceutical agent, preferably a low solubility and/or low dissolution rate solubilizing agent, more preferably topiramate and a solubilizing agent, preferably surfactant; and a push layer comprising an osmopolymer; (b) a semi-permeable wall surrounding said core and (c) an exit orifice through the semi-permeable wall for releasing the pharmaceutical agent from the dosage form over a prolonged period of time.

One skilled in the art will recognize will that wherein the dosage forms of the present invention comprise a first drug composition comprising a pharmaceutical agent and a solubilizing agent; and a second drug composition comprising a pharmaceutical agent and a solubilizing agent; then the pharmaceutcal agent in the first and second drug compositions may be the same or different and the solubilizing agent on the first and second drug compostions my be the same or different. One skilled in the art will further recognize that additional, optional components within the first and second drug compositions, for example structural polymer, binder, lubricant, and the like, when present in both the first and second drug compositions may similarly be the same or different.

The formulations and processes for the manufacture of the push layer 40, the semi-permeable wall 20 and the exit orifice(s) 60 are well known in the art. The components and processes for the manufacture of the push layer, semi-permeable wall and exit orifice(s) are also briefly described below.

Push layer 40 comprises a displacement composition in contacting, layered arrangement with drug composition 30 as illustrated in FIG. 4. Wherein more than one drug composition is present in the dosage form (as in FIG. 6), the push layer 40 is preferably in contacting, layered arrangement with only one of the drug compositions.

In an embodiment of the present invention push layer 40 comprises an osmopolymer. In another embodiment of the present invention, push layer 40 comprises an osmopolymer and an osmoagent.

Push layer 40 comprises osmopolymer 41 that imbibes water and swells to push the drug composition of the drug layer(s) through the exit orifice of the dosage form. The osmopolymers are swellable, hydrophilic polymers that interact with water and swell or expand to a high degree, typically exhibiting a 2-50 fold volume increase. The osmopolymer can be non-crosslinked or crosslinked. Preferably, push layer 40 comprises from about 20 to about 375 mg of osmopolymer 41, represented by “V” symbols in FIG. 4.

Wherein osmopolymers are present in both a drug composition and the push layer, the osmopolymer 41 in the push layer 40 possesses a higher molecular weight than the osmopolymer in drug composition. For example, such a situation may be found wherein the structural polymer in the drug composition is an osmopolymer.

Representatives of osmopolymers (i.e. fluid-imbibing displacement polymers) comprise members selected from poly(alkylene oxide) of 1 million to 15 million number-average molecular weight, as represented by poly(ethylene oxide), and poly(alkali carboxymethylcellulose) of 500,000 to 3,500,000 number-average molecular weight, wherein the alkali is sodium, potassium or lithium. Examples of alternate osmopolymers comprise polymers that form hydrogels, such as CARBOPOL® acidic carboxypolymer, a polymer of acrylic cross-linked with a polyallyl sucrose, also known as carboxypolymethylene, and carboxyvinyl polymer having a molecular weight of 250,000 to 4,000,000; CYANAMER® polyacrylamides; cross-linked water swellable indenemaleic anhydride polymers; GOOD-RITE® polyacrylic acid having a molecular weight of 80,000 to 200,000; AQUA-KEEPS® acrylate polymer polysaccharides composed of condensed glucose units, such as diester cross-linked polygluran; and the like. Representative polymers that form hydrogels are known to the prior art in U.S. Pat. No. 3,865,108, issued to Hartop; U.S. Pat. No. 4,002,173, issued to Manning; U.S. Pat. No. 4,207,893, issued to Michaels; and in Handbook of Common Polymers, Scott and Roff, Chemical Rubber Co., Cleveland, Ohio.

Push layer 40 further, optionally, comprises an osmotically effective compound, osmoagent 42, represented by large circles in FIG. 4. Preferably, the osmoagent 42 comprises up to about 40% by weight of the push layer, more preferably, from about 5% to about 30% by weight of the push layer, more preferably still, from about 10% to about 30% by weight of of the push layer. Osmotically effective compounds are known also as osmoagents and/or as osmotically effective solutes. Preferably, push layer 40 comprises an osmoagent.

Osmoagents 42, which may be found in the drug composition and/or the push layer in the dosage forms of the present invention are those that exhibit an osmotic activity gradient across the wall 20. Suitable osmoagents include, but are not limited to, sodium chloride, potassium chloride, lithium chloride, magnesium sulfate, magnesium chloride, potassium sulfate, sodium sulfate, lithium sulfate, potassium acid phosphate, mannitol, urea, inositol, magnesium succinate, tartaric acid, raffinose, sucrose, glucose, lactose, sorbitol, inorganic salts, organic salts, carbohydrates, and the like.

Push layer 40 may further optionally comprises a pharmaceutically acceptable binder 43, such as a vinyl polymer, represented by triangles in FIG. 4. The vinyl polymer comprises a 5,000 to 350,000 viscosity-average molecular weight, represented by a member selected from the group consisting of poly-n-vinylamide, poly-n-vinylacetamide, poly(vinyl pyrrolidone), also known as poly-n-vinylpyrrolidone, poly-n-vinylcaprolactone, poly-n-vinyl-5-methyl-2-pyrrolidone, and poly-n-vinylpyrrolidone copolymers with a member selected from the group consisting of vinyl acetate, vinyl alcohol, vinyl chloride, vinyl fluoride, vinyl butyrate, vinyl laureate, and vinyl stearate. Push layer 40 preferably contains from about 0.01 to about 25 mg of vinyl polymer.

Push layer 40 may further optionally comprise from 0 to about 5 mg of a nontoxic colorant or dye 46, identified by vertical wavy lines in FIG. 2. Suitable examples of colorant or dye 46 include Food and Drug Administration Colorants (FD&C), such as FD&C No.1 blue dye, FD&C No. 4 red dye, red ferric oxide, yellow ferric oxide, titanium dioxide, carbon black, indigo, and the like.

Push layer 40 may further optionally comprise lubricant 44, identified by half circles in FIG. 4. Suitable examples include, but are not limited to, a member selected from the group consisting of sodium stearate, potassium stearate, magnesium stearate, stearic acid, calcium stearate, sodium oleate, calcium palmitate, sodium laurate, sodium ricinoleate and potassium linoleate, and blends of such lubricants. The amount of lubricant included in the push layer 40 is preferably in the range of from about 0.01 to about 10 mg.

Push layer 40 may further optionally comprise an antioxidant 45, represented by slanted dashes in FIG. 4, wherein the antioxidant is present to inhibit the oxidation of ingredients within the push layer. Push layer 40 comprises from 0.0 to about 5 mg of an antioxidant. Representative antioxidants include, but are not limited to, ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiary butylphenol, alpha-tocopherol, and propylgallate.

Semi-permeable wall 20, sometimes also referred to as a membrane, is formed to be permeable to the passage of external water. Semi-permeable wall 20 is also substantially impermeable to the passage of the components of the drug composition and push layer, such as drug, solubilizing agent, structural polymer, osmagent, osmopolymer and the like. As such, wall 20 is semi-permeable. The selectively semi-permeable compositions used for forming the semi-permeable wall 20 are essentially non-erodible and are substantially insoluble in biological fluids during the life of the dosage form.

Representative polymers suitable for forming semi-permeable wall 20 comprise semi-permeable homopolymers, semi-permeable copolymers, and the like. Such materials include, but are not limited to, cellulose esters, cellulose ethers and cellulose ester-ethers. The cellulosic polymers have a degree of substitution (DS) of their anhydroglucose unit of from greater than 0 up to 3, inclusive. Degree of substitution (DS) means the average number of hydroxyl groups originally present on the anhydroglucose unit that are replaced by a substituting group or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkysulfamate, semi-permeable polymer forming groups, and the like, wherein the organic moieties contain from one to twelve carbon atoms, and preferably from one to eight carbon atoms.

Semi-permeable wall 20 may further compromise a semi-permeable polymer selected from the group consisting of cellulose acylate, cellulose diacylate, cellulose triacylate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers include cellulose acetate having a DS in the range of about 1.8 to about 2.3 and an acetyl content in the range of about 32 to about 39.9%; cellulose diacetate having a DS in the range of about 1 to about 2 and an acetyl content in the range of about 21 to about 35%; cellulose triacetate having a DS in the range of about 2 to about 3 and an acetyl content in the range of about 34 to about 44.8%; and the like. Preferred cellulosic polymers include cellulose propionate having a DS of about 1.8 and a propionyl content of about 38.5%; cellulose acetate propionate having an acetyl content in the range of about 1.5 to about 7% and an acetyl content in the range of about 39% to about 42%; cellulose acetate propionate having an acetyl content in the range of about 2.5% to about 3%, an average propionyl content in the range of about 39.2% to about 45%, and a hydroxyl content in the range of about 2.8% to about 5.4%; cellulose acetate butyrate having a DS of about 1.8, an acetyl content in the range of about 13% to about 15%, and a butyryl content in the range of about 34% to about 39%; cellulose acetate butyrate having an acetyl content in the range of about 2% to about 29%, a butyryl content in the range of about 17% to about 53%, and a hydroxyl content in the range of about 0.5% to about 4.7%; cellulose triacylates having a DS in the range of about 2.6 to about 3, such as cellulose trivalerate, cellulose trilamate, cellulose tripalmitate, cellulose trioctanoate and cellulose tripropionate; cellulose diesters having a DS in the range of about 2.2 to about 2.6, such as cellulose disuccinate, cellulose dipalmitate, cellulose dioctanoate, cellulose dicaprylate, and the like; and mixed cellulose esters, such as cellulose acetate valerate, cellulose acetate succinate, cellulose propionate succinate, cellulose acetate octanoate, cellulose valerate palmitate, cellulose acetate heptanoate, and the like. Semi-permeable polymers are known in U.S. Pat. No.4,077,407, and they can be synthesized by procedures described in Encyclopedia of Polymer Science and Technology, Vol. 3, pp. 325-354 (1964), Interscience Publishers Inc., New York, N.Y.

Additional semi-permeable polymers that may be used for forming semi-permeable wall 20 comprise cellulose acetaldehyde dimethyl acetate; cellulose acetate ethylcarbamate; cellulose acetate methyl carbamate; cellulose dimethylaminoacetate; semi-permeable polyamide; semi-permeable polyurethanes; semi-permeable sulfonated polystyrenes; cross-linked selectively semi-permeable polymers formed by the coprecipitation of an anion and a cation, as disclosed in U.S. Pat. Nos. 3,173,876; 3,276,586; 3,541,005; 3,541,006 and 3,546,142; semi-permeable polymers, as disclosed by Loeb, et al. in U.S. Pat. No. 3,133,132; semi-permeable polystyrene derivatives; semi-permeable poly(sodium styrenesulfonate); semi-permeable poly(vinylbenzyltrimethylammonium chloride); and semi-permeable polymers exhibiting a fluid permeability of 10⁻⁵ to 10⁻² (cc. mil/cm hr.atm), expressed as per atmosphere of hydrostatic or osmotic pressure differences across a semi-permeable wall. The polymers are known to the art in U.S. Pat. Nos. 3,845,770; 3,916,899 and 4,160,020; and in Handbook of Common Polymers, Scott and Roff (1971) CRC Press, Cleveland, Ohio. Wall 20 can optionally be formed as two or more lamina such as described in U.S. Pat. No. 6,210,712.

Preferably, the semi-permeable wall 20 comprises a polymer selected from the group consisting of cellulose acetate and cellulose acetate butyrate.

Semi-permeable wall 20 may further, optionally, comprise a flux-regulating agent. The flux regulating agent is a compound added to assist in regulating the water permeability or flux through semi-permeable wall 20. The flux-regulating agent can be a flux-enhancing agent or a flux-decreasing agent. The flux-regulating agent can therefore be pre-selected to increase or decrease the flux of the external water through the semi-permeable membrane. Flux-regulating agents that produce a marked increase in permeability to fluid such as water are often essentially hydrophilic, while those that produce a marked decrease to fluids such as water are essentially hydrophobic. The amount of flux-regulator in semi-permeable wall 20 when incorporated therein is preferably in the range of from about 0.01% to about 25% by weight or more.

Suitable flux-regulating agents include, but are not limited to, polyhydric alcohols, polyalkylene glycols, polyalkylenediols, polyesters of alkylene glycols, and the like.

Flux enhancers include, but are not limited to, polyethylene glycol 300, 400, 600, 1500, 4000, 6000 and the like; low molecular weight glycols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glycol dipropionate, glycerol acetate esters, and the like. Preferred flux enhancers include the group of difunctional block-copolymer of ethylene oxide and propylene oxide conforming to the general formula OH(C₂H₄O)ₐ(C₃H₆O)_{b}(C₂H₄O)H, known as PLURONIC® co-polymers (sold in pharmaceutical grade under the trade name LUTROL).

Flux-decreasing agents include, but are not limited to, phthalates substituted with an alkyl or alkoxy or with both an alkyl and alkoxy group such as diethyl phthalate, dimethoxyethyl phthalate, dimethyl phthalate, and [di(2-ethylhexyl) phthalate], aryl phthalates such as triphenyl phthalate, and butyl benzyl phthalate; polyvinyl acetates, triethyl citrate, Eudragit; insoluble salts such as calcium sulfate, barium sulfate, calcium phosphate, and the like; insoluble oxides such as titanium oxide; polymers in powder, granule and like form such as polystyrene, polymethylmethacrylate, polycarbonate, and polysulfone; esters such as citric acid esters esterified with long chain alkyl groups; inert and substantially water impermeable fillers; resins compatible with cellulose based wall forming materials, and the like.

Other materials may be further, optionally, included in the semi-permeable wall composition for imparting flexibility and/or elongation properties, i.e. to make semi-permeable wall 20 less brittle and/or to render tear strength to semi-permeable wall 20. Suitable materials include, but are not limited to, phthalate plasticizers such as dibenzyl phthalate, dihexyl phthalate, butyl octyl phthalate, straight chain phthalates of six to eleven carbons, di-isononyl phthalte, di-isodecyl phthalate, and the like. Plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in semi-permeable wall 20 when incorporated therein is preferably in the range of from about 0.01% to about 20% weight, or higher.

Exit orifice 60 is provided in each osmotic dosage form. Exit 60 may encompass one or more exit orifices. Exit 60 cooperates with the drug composition(s) within the dosage form for the uniform release of drug from the dosage form. The exit can be provided during the manufacture of the dosage form or during drug delivery by the dosage form in a fluid environment of use.

Exit 60 may include an orifice that is formed or formable from a substance or polymer that erodes, dissolves or is leached from the outer wall to thereby form an exit orifice. The substance or polymer may include, for example, an erodible poly(glycolic) acid or poly(lactic) acid in the semi-permeable wall; a gelatinous filament; a water-removable poly(vinyl alcohol); a leachable compound, such as a fluid removable pore-former selected from the group consisting of inorganic and organic salt, oxide, carbohydrate, and the like.

The exit 60, or a plurality of exits, can alternatively be formed by leaching a member selected from the group consisting of sorbitol, lactose, fructose, glucose, mannose, galactose, talose, sodium chloride, potassium chloride, sodium citrate and mannitol to provide a uniform-release dimensioned pore-exit orifice.

Exit 60 can have any shape, such as round, triangular, square, oval, elliptical, and the like, for the uniform metered dose release of a drug from the dosage form.

When more than one exit orifice is present in the dosage form, the exits may be present in spaced-apart relation on one or more surfaces of the dosage form, provided that at least one of the exit orifices is situated such that they expose drug composition to the external environment.

The drug compositions of the present invention may be prepared according to known methods, for example as a granulation, as a dry blend, as a co-precipitate, as a roller compacted blend, and the like. Preferably, the drug composition is prepared as a granulation.

A variety of processing techniques can be used to promote uniformity of mixing between the pharmaceutical agent 31 and solubilizing agent, preferably surfactant, 33 in drug composition 30. In one method, the drug and surfactant are each micronized to a nominal particle size of less than about 200 microns, preferably, to a nominal particle size of less than about 100 microns, more preferably, to a nominal particle size of less than about 50 microns. Standard micronization processes such as jet milling, cryogrinding, bead milling, and the like, may be used.

Alternatively, the drug and solubilizing agent may be dissolved in a common solvent to produce mixing at the molecular level and co-dried to a uniform mass. The resulting mass may be ground and sieved to a free-flowing powder. The resulting free-flowing powder may be further, optionally, granulated with wet mass sieving or fluid bed granulation with any optional structural polymer to form a drug composition (in the form of a granulation) of the present invention.

Alternatively still, pharmaceutical agent 31 and solubilizing agent 33 may be melted together at elevated temperature to mix the drug in solubilizing agent, preferably surfactant, and then congealed to room temperature. The resulting solid may be ground, sized, and optionally, further granulated with structural polymer.

In yet another manufacturing process, pharmaceutical agent 31 and solubilizing agent 33 may be dissolved in a common solvent or blend of solvents and spray dried to form a co-precipitate that is then further, optionally incorporated with structural polymer by standard granulation processing by fluid bed processing or wet mass sieving.

In yet another manufacturing process, pharmaceutical agent 31 and solubilizing agent 33 may be dissolved in a common solvent or blend of solvents which pharmaceutical agent/surfactant solution is then sprayed onto the optional structural polymer directly in a fluid bed granulation process.

The drug composition of the present invention may then be formulated into the dosage forms of the present invention. Drug composition 30 within the dosage form is preferably formed by compression of the pharmaceutical agent 31, solubilizing agent 33, preferably surfactant, and if present, the structural polymer 32. For the preparation of osmotic dosage forms, one or more drug compositions are compressed in a stacked orientation, with a push layer prepared and incorporated into the dosage form in contacting relation to at least one of the drug compositions.

Each drug composition is prepared by mixing the pharmaceutical agent 31 with the solubilizing agent 33 and any additional components (e.g. structural polymer 32) into a uniform mixture.

Alternatively, the drug composition 30 may be formed from particles by comminution that produces the size of the pharmaceutical agent and the size of any accompanying polymers used in the fabrication of the drug composition, typically as a core containing the compound. Means for producing such particles include, but are not limited to, granulation, spray drying, sieving, lyophilization, crushing, grinding, jet milling, micronizing and chopping to produce the intended micron particle size. The process can be performed by size reduction equipment, such as a micropulverizer mill, a fluid energy grinding mill, a grinding mill, a roller mill, a hammer mill, an attrition mill, a chaser mill, a ball mill, a vibrating ball mill, an impact pulverizer mill, a centrifugal pulverizer, a coarse crusher, a fine crusher, and the like. The size of the particle(s) can be ascertained by screening, including a grizzly screen, a flat screen, a vibrating screen, a revolving screen, a shaking screen, an oscillating screen, a reciprocating screen and the like. The processes and equipment for preparing drug and/or carrier particles are disclosed in Remington's Pharmaceutical Sciences, 18th Ed., pp.1615-1632 (1990); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Hixon, pp. 94-103 (1990).

Exemplary solvents suitable for manufacturing drug compositions and/or the push layer for the dosage form comprise aqueous or inert organic solvents that do not adversely harm the materials used in the system. Such solvents include, but are not limited to, members selected from the group consisting of aqueous solvents, alcohols, ketones, esters, ethers, aliphatic hydrocarbons, halogenated solvents, cycloaliphatics, aromatics, heterocyclic solvents and mixtures thereof. Suitable examples of solvents include, but are not limited to, acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride nitroethane, nitropropane tetrachloroethane, ethyl ether, isopropyl ether, cyclohexane, cyclooctane, benzene, toluene, naphtha, tetrahydrofuran, diglyme, water, aqueous solvents containing inorganic salts such as sodium chloride, calcium chloride, and the like, and mixtures thereof such as acetone and water, acetone and methanol, acetone and ethyl alcohol, methylene dichloride and methanol, and ethylene dichloride and methanol.

Push layer 40 may be similarly prepared according to known methods, for example according to the processes described above, by mixing the appropriate ingredients under appropriate conditions (e.g. osmoagent, omsopolymer, etc.).

Semi-permeable wall 20 may be similarly perpeared accoding to known methods, for example by pan coating, by mixing the appropriate ingredients and applying the resulting mixture to dosage form.

Dosage form components (e.g. drug composition(s), push layer, semi-permebale wall, exit orifice, etc.) may be combined to form the dosage forms of the present invention according to standard techniques known in the art. More specifically, the dosage form core, comprising one or more drug compositions, and when present the push layer, is prepared first, preferably by compression. The semi-permeable wall is then coated onto the core and one or more exit orifices are provided through the semi-permeable wall to expose one or more drug compositions to the external environment.

For example, the dosage form may be manufactured by the wet granulation technique. In the wet granulation technique, the drug, optional structural polymer and solubilizing agent, preferably surfactant, are blended using an organic solvent, such as denatured anhydrous ethanol, as the granulation fluid. Any additional excipients can then be dissolved in a portion of the granulation fluid, such as the solvent described above, and this latter prepared solution is slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven. The dried granules are then sized. Next, magnesium stearate, or another suitable lubricant, is added to the drug granulation, and the granulation is put into milling jars and mixed on a jar mill for up to 10 minutes. The composition is pressed into a layer, for example, in a Manesty® press or a Korsch LCT press.

For a bi-layered core (i.e. a dosage form which comprises a drug composition and a push layer), the drug composition is pressed and a similarly prepared granulation of the push layer is pressed against the drug composition. This intermediate compression typically takes place under a force of about 50-100 newtons. Final stage compression typically takes place at a force of 3500 newtons or greater, often 3500-5000 newtons.

Wherein the core comprises two or more drug compositions and a push layer, each drug composition, prepared as described above is individually compressed. The push layer is then pressed against at least one of the drug compositions, in an intermediate compression step as described above. Final compression of the multi-layer core is then applied as described above.

Single, bi-layer or multi-layer compressed cores are then fed to a dry coater press, e.g., Kilian® Dry Coater press, and subsequently coated with the semi-permeable wall materials, according to known methods.

In another process of manufacture the drug and other ingredients comprising the drug composition are blended and pressed into a solid layer. The layer possesses dimensions that correspond to the internal dimensions of the area the layer is to occupy in the dosage form, and it also possesses dimensions corresponding to the push layer, if included, for forming a contacting arrangement therewith. The drug and other ingredients can also be blended with a solvent and mixed into a solid or semisolid form by conventional methods, such as ballmilling, calendering, stirring or rollmilling, and then pressed into a. preselected shape. Next, if included, the push layer components are placed in contact with the drug composition in a like manner. The layering of the drug composition(s) and the push layer can be fabricated by conventional two-layer press techniques. The compressed cores may then be coated with the semi-permeable wall material, according to known methods.

Another manufacturing process that can be used comprises blending the powdered ingredients for each layer in a fluid bed granulator. After the powdered ingredients are dry blended in the granulator, a granulating fluid, for example, poly(vinylpyrrolidone) in water, is sprayed onto the powders. The coated powders are then dried in the granulator. This process granulates all the ingredients present therein while adding the granulating fluid. After the granules are dried, a lubricant, such as stearic acid or magnesium stearate, is mixed into the granulation using a blender e.g., V-blender or tote blender. The granules are then pressed in the manner described above.

Pan coating may be conveniently used to provide semi-permeable wall 20 of the completed osmotic dosage forms. In the pan coating system, the wall-forming composition (comprising the semi-permeable polymer and optional, additional materials) is deposited by successive spraying of the appropriate wall composition onto the compressed single, bi-layered or mulit-layered core (which ore comprises the drug layer(s) and, where present, the push layer), accompanied by tumbling in a rotating pan. A pan coater is often used because of its availability at commercial scale.

Other known coating techniques may alternatively be used for coating the compressed core. For example, semi-permeable wall 20 of the dosage form may be formed in one technique using the air-suspension procedure. This procedure consists of suspending and tumbling the compressed single, bi-layer or multi-layer core in a current of warmed air and the semi-permeable wall forming composition, until the semi-permeable wall is applied to the core. The air-suspension procedure is well suited for independently forming the semi-permeable wall of the dosage form. The air-suspension procedure is described in U.S. Pat. No. 2,799,241; in J. Am. Pharm. Assoc., Vol. 48, pp. 451-459 (1959); and, ibid., Vol. 49, pp. 82-84 (1960). The dosage form may alternatively be coated with a Wurster® air-suspension coater using, for example, methylene dichloride methanol as a cosolvent for the wall forming material. An Aeromatic® air-suspension coater may alternatively be used employing a suitable co-solvent.

Once coated, semi-permeable wall 20 is dried in a forced-air oven or in a temperature and humidity controlled oven to free the dosage form of any solvent(s) used in the manufacturing. Drying conditions are conventionally chosen on the basis of available equipment, ambient conditions, solvents, coatings, coating thickness, and the like.

Preferably, the drug compositions, the push layer and/or the dosage forms are dried to remove volatile organic and in-organic solvents to levels that are pharmaceutically acceptable and/or optimal for manufacturing. More preferably, the drug compositions, the push layer and/or the dosage forms are to less than about 10% moisture, more preferably still, to less than about 5% moisture, most preferably less than about 3% moisture.

One or more exit orifices are provided according to known methods, for example by drilling, in the drug composition end of the dosage form. Alternatively, one or more exit orifices may be provided in the drug composition end of the dosage form by erosion or leaching.

The dosage form can therefore be constructed with one or more exits in spaced-apart relation on one or more surfaces of the dosage form.

Drilling, including mechanical and laser drilling, through the semi-permeable wall can be used to form the exit orifice. Such exits and equipment for forming such exits are disclosed in U.S. Pat. No. 3,916,899, by Theeuwes and Higuchi and in U.S. Pat. No.4,088,864, by Theeuwes, et al.

Leachable or eroable exit orfices may be formed or formable from a substance or polymer that erodes, dissolvces or is leached from the outer semi-permeable (outer) wall to thereby form an exit orifice. The substance or polymer may include for example, an erodible poly(glucolic)acid or poly(lactic)acid in the semi-permeable wall, a gelatinous filament, a water removable poly(vinyl)alcohol, a leachable compound such as a fluid removable pore former, for exa,pel an inorganic or organic salt, oxide or carbohydrate. The exit or plurality of exits can be formed by leaching a member selected from the group consisting of sorbitol, lactose, fructose, glucose, mannose, galactose, talose, sodium chloride, potassium chloride, sodium citrate and mannitol to provide a uniform release dimensioned pore exit orifice. The exit can have any shape, such as, round, triangular, square, elliptical, and the like.

The dosage form may be further, optionally coated with additional water soluble overcoats, which may be colored (e.g., OPADRY colored coatings) or clear (e.g., OPADRY Clear).

The dosage form may further, optionally comprise a smoothing coat, which smoothing coat is applied to the compressed drug core, according to known methods, prior to the application of the semi-permeable wall. Suitable examples of formulations and components which may used in the smoothing coat include, but are not limited to, hydroxypropylcellulose, hydroxyethylcellulose, methylcellulose, hydroxypropyl methylcellulose, and the like. The coating may further optionally contain polyethylene glycol of 400 to 6000 molecular weight, polyvinyl pyrrolidone of 2500 to 1,000,000 molecular weight, and the like.

The dosage forms of the present invention provide controlled release of pharmaceutical agent, preferably topiramate, over a prolonged period of time, preferably, for greater than about 1 hour, more preferably, for at least about 4 hours, more preferably still, for at least about 8 hours, more preferably, for at least about 10 hours, more preferably still, for at least about 14 hours, more preferably still, for at least 18 hours, more preferably still, for at least 20 hours, more preferably still for at least 22 hours, more preferably still for up to about 24 hours. Preferably, the dosage forms of the present invention provide controlled release of pharmaceutical agent for about 2 to about 24 hours, more preferably, for about 4 to about 24 hours.

In an embodiment of the present invention, the release of drug from the dosage forms of the present invention provides efficacious therapy for about 24 hours. In another embodiment of the present invention, the dosage form releases drug for about 16 to about 24 hours after administration.

In an embodiment of the present invention, the dosage form comprises an optional immediate release drug overcoat which provides immediate drug delivery (i.e. within less than about 1 hour after administration) and controlled drug delivery continuing thereafter until the dosage form ceases to release drug, preferably, at least about 8 hours, more preferably, about 12 hours, more preferably still, about 16 hours, more preferably still about 18 hours, more preferably still, about 22 hours, more preferably still, about 24 hours.

Representative dosage forms of the present invention exhibit T₇₀ values of greater than about 8 hours, preferably, greater than about 10 hours, more preferably, greater than about 12 hours, more preferably still, greater than about 16 hours, and release drug, preferably topiramate, for a continuous period of time of more than about 12 hours, more preferably, for more than about 16 hours, more preferably still, for about 24 hours.

Within about 2 hours following administration, representative dosage forms of the present invention release drug, preferably topiramate, at a substantially zero order rate of release or at a substantially ascending rate of release, depending upon the composition of drug composition(s) and push layers. Preferably, drug release continues for a prolonged period of time. Following the prolonged period of delivery, drug continues to be delivered for several more hours until the dosage form is spent or expelled from the GI tract.

In a bi-layer embodiment of once-a-day dosage forms in accord with the present invention, the dosage forms have a T₇₀ of about 15 hours to about 18 hours, preferably, about 17 hours, and provided release of drug, preferably topiramate, for a continuous period of time, preferably, for at least about 24 hours. Preferably, the dosage form releases drug with a substantially zero order rate of release.

In a tri-layer embodiment of the present invention, the dosage form of the present invention comprises two drug compositions and a push layer, wherein the amount and/or concentration of drug in the first drug composition is less than the amount and/or concentration of drug in the second drug composition. Representative tri-layer dosage forms of the present invention exhibit T₇₀ values of greater than about 8 hours, preferably, greater than about 12 hours, more preferably, greater than about 14 hours, and release drug, preferably topiramate, for a continuous period of time of more than about 16 hours, preferably for about 24 hours. Preferably, the dosage form releases drug with a substantially ascending rate of release.

In an embodiment of the present invention, the dosage forms of the present invention release the pharmaceutical agent (drug) at various rates of release between about 1%/hr and about 12%/hr over a prolonged period of time.

In an embodiment of the present invention, the dosage forms release pharmaceutical agent with a substantially zero order rate of release. In another embodiment of the present invention, the dosage forms release pharmaceutical agent with a substantially ascending rate of release. In yet another embodiment of the present invention, the dosage forms release pharmaceutical agent with a release rate which results in a substantially ascending drug plasma concentration.

The present invention is further directed to a method of treatment comprising administering any of the drug compositions or dosage forms of the present invention, to a patient in need thereof. Said drug compositions and/or dosage forms comprise pharmaceutical agent, preferably topiramate, in the range of from about 1 mg to about 750 mg.

The method, in one embodiment, comprises administering orally to a patient in need thereof, a pharmaceutical agent, preferably topiramate, administered from a dosage form comprising the desired amount of said pharmaceutical agent and solubilizing agent, preferably surfactant.

The present invention further provides methods for administering pharmaceutical agent, preferably topiramate, to a patient, and methods for producing a desired drug plasma concentration of topiramate. In an embodiment of the present invention is a method for administering orally to a patient in need thereof, a dosage form that administers at a controlled rate, over a continuous period of time up to about 24 hours, drug for its intended therapy. In another embodiment of the present invention, the method comprises administering orally to a patient in need thereof, a therapeutic dose of pharmaceutical agent, preferably topiramate, from a single dosage form that administers the topiramate over about 24 hours.

The present invention is further directed to a method of treatment comprising administering to a patient in need thereof, an oral controlled release dosage form of a pharmaceutical agent, preferably topiramate, wherein the pharmaceutical agent is released from the dosage form in a substantially zero order rate of release.

The present invention is further directed to a method of treating comprising administering to a patient in need thereof, an oral controlled release dosage form of a pharmaceutical agent, preferably topiramate, wherein the pharmaceutical agent is released from the dosage form in a substantially ascending rate of release.

The present invention is further directed to a method of treating comprising administering to a patient in need thereof, an oral controlled release dosage form of a pharmaceutical agent, preferably topiramate, wherein the pharmaceutical agent is released from the dosage form at a rate which results in a substantially ascending drug plasma concentration.

The present invention is further directed to a method of treating a disorder is selected from the group consisting of epilepsy, migraine, glaucoma and other ocular disorders (including diabetic retinopathy), essential tremor, restless limb syndrome, obesity, weight loss, Type II Diabetes Mellitus, Syndrome X, impaired oral glucose tolerance, diabetic skin lesions, cluster headaches, neuralgia, neuropathic pain (including diabetic neuropathy), elevated blood glucose levels, elevated blood pressure, elevated lipids, bipolar disorder, dementia, depression, psychosis, mania, anxiety, schizophrenia, OCD, PTSD, ADHD, impulse control disorders (including bulimia, binge eating, substance abuse, etc.), ALS, asthma, autism, autoimmune disorders (including psoriasis, rheumatoid arthritis, etc.), chronic neurodegenerative disorders, acute neurodegeneration, sleep apnea and other sleep disorders or for promoting wound healing, comprising administering to a patient in need thereof, any of the drug compositions or dosage forms of the present invention.

Preferably, the disorder is selected from the group consisting of epilepsy, migraine, diabetic retinopathy, diabetic neuropathy, diabetic skin lesions, obesity, weight loss, Type II Diabetes Mellitus, Syndrome X, impaired oral glucose tolerance, elevated blood glucose levels and elevated blood pressure.

In an embodiment, the push layer comprises a displacement composition in contacting layered arrangement with the tablet core as shown in FIG. 2. The push layer comprises an osmopolymer that imbibes an aqueous or biological fluid and swells to push the drug composition through the exit port through the semipermeable membrane. A polymer having suitable imbibition properties may be referred to herein as an osmopolymer. The osmopolymers are swellable, hydrophilic polymers that interact with water and aqueous biological fluids and swell or expand to a high degree, typically exhibiting a 2-50 fold volume increase. The osmopolymer can be non-crosslinked or crosslinked.

Preferably, the push layer comprises about 20 to about 375 mg of osmopolymer. Preferably, the osmopolymer in the push layer possesses a higher molecular weight than the osmopolymer in drug layer.

Representatives of fluid-imbibing displacement polymers comprise members selected from poly(alkylene oxide) of 1 million to 15 million number-average molecular weight, as represented by poly(ethylene oxide), and poly(alkali carboxymethylcellulose) of 500,000 to 3,500,000 number-average molecular weight, wherein the alkali is sodium, potassium or lithium. Examples of additional polymers for the formulation of the push-displacement composition comprise osmopolymers comprising polymers that form hydrogels, such as Carbopol® acidic carboxypolymer, a polymer of acrylic cross-linked with a polyallyl sucrose, also known as carboxypolymethylene, and carboxyvinyl polymer having a molecular weight of 250,000 to 4,000,000; Cyanamer® polyacrylamides; cross-linked water swellable indenemaleic anhydride polymers; Good-rite® polyacrylic acid having a molecular weight of 80,000 to 200,000; Aqua-Keeps® acrylate polymer polysaccharides composed of condensed glucose units, such as diester cross-linked polygluran; and the like. Representative polymers that form hydrogels are known to the prior art in U.S. Pat. No. 3,865,108, issued to Hartop; U.S. Pat. No. 4,002,173, issued to Manning; U.S. Pat. No.4,207,893, issued to Michaels; and in Handbook of Common Polymers, Scott and Roff, Chemical Rubber Co., Cleveland, Ohio.

In an embodiment, the push layer comprises 0 to about 75 mg, and preferably, about 5 to about 75 mg of an osmotically effective compound, an osmoagent. The osmotically effective compounds are known also as osmoagents and as osmotically effective solutes. The osmoagent that may be found in the drug layer and the push layer in the dosage form are those that exhibit an osmotic activity gradient across the semipermeable membrane. Suitable osmoagents comprise a member selected from the group consisting of sodium chloride, potassium chloride, lithium chloride, magnesium sulfate, magnesium chloride, potassium sulfate, sodium sulfate, lithium sulfate, potassium acid phosphate, mannitol, urea, inositol, magnesium succinate, tartaric acid, raffinose, sucrose, glucose, lactose, sorbitol, inorganic salts, organic salts and carbohydrates.

The push layer may further comprises a therapeutically acceptable vinyl polymer. The vinyl polymer comprises a 5,000 to 350,000 viscosity-average molecular weight, represented by a member selected from the group consisting of poly-n-vinylamide, poly-n-vinylacetamide, poly(vinyl pyrrolidone), also known as poly-n-vinylpyrrolidone, poly-n-vinylcaprolactone, poly-n-vinyl-5-methyl-2-pyrrolidone, and poly-n-vinylpyrrolidone copolymers with a member selected from the group consisting of vinyl acetate, vinyl alcohol, vinyl chloride, vinyl fluoride, vinyl butyrate, vinyl laureate, and vinyl stearate. Push layer preferably comprises about 0.01 to about 25 mg of vinyl polymer.

The push layer may further comprise 0 to 5 mg of a nontoxic colorant or dye. The colorant includes Food and Drug Administration Colorant (FD&C), such as FD&C No.1 blue dye, FD&C No. 4 red dye, red ferric oxide, yellow ferric oxide, titanium dioxide, carbon black, and indigo.

The push layer may further comprise a lubricant. Typical lubricants comprise a member selected from the group consisting of sodium stearate, potassium stearate, magnesium stearate, stearic acid, calcium stearate, sodium oleate, calcium palmitate, sodium laurate, sodium ricinoleate and potassium linoleate, and blends of such lubricants. In an embodiment, the amount of lubricant included in the push layer 40 is about 0.01 to about 10 mg.

The push layer may further comprise an antioxidant to inhibit the oxidation of ingredients comprising expandable formulation. In an embodiment, the push layer comprises 0.00 to about 5 mg of an antioxidant. Representative antioxidants comprise a member selected from the group consisting of ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiary butylphenol, alpha-tocopherol, and propylgallate.

The controlled release topiramate dosage form may be composed of coated granules comprising both topiramate and a solubilizing agent, preferably a surfactant. The coated granules may further comprise a viscosity agent or a binding agent or both.

One advantage of the present invention is the ability to modify the content and amount of the granule coating and granule composition to modify the release rate of topiramate from the dosage form.

For example, the composition of the controlled release topiramate dosage form may be selected by which the release rate of topiramate is at its maximum value for at least two hours in an in vitro release rate assay.

Alternatively, the composition of the controlled release topiramate dosage form may be selected by which the release rate of topiramate is at its maximum value for at least three hours, four hours, six hours, eight hours, ten hours or at least 12 hours in an in vitro release rate assay.

Alternatively still, the composition of the controlled release topiramate dosage form may be selected by which the maximum release rate of topiramate is reached after about 1 hour, 1.5 hours, 2 hours, 2.5 hours, 3 hours, 4 hours, 5 hours, 6.5 hours, 8 hours, 10 hours, 12 hours, 14 hours or 16 hours in an in vitro release rate assay.

### EXEMPLIFIED DESCRIPTION OF THE INVENTION

The following examples are illustrative of the present invention and they should not be considered as limiting the scope of the invention in any way, as these examples and other equivalents thereof will become apparent to those versed in the art in light of the present disclosure, drawings and accompanying claims.

### Example 1

For purposes of comparison a conventional topiramate drug formulation was prepared as follows.

First, a binder solution was prepared. 3 kg of polyvinylpyrrolidone (K29-32; average molecular weight of 40,000) was dissolved in 17 kg of water. Then, the 17.28 kg of Topiramate, 5.75 kg of Polyethylene oxide N-80 (approximately 2 million molecular weight) and 32.16 kg of Poloxamer 407 and 1.5 kg of polyvinylpyrrolidone identified as K29-32 were added to a fluid bed granulator bowl. The dry materials were fluidized and mixed while 10 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 12 g of butylated hydroxytoluene and lubricated with 1.2 kg of stearic acid and 600 g of magnesium stearate.

FIG. 1 shows the smearing observed with the conventional formulation without the use of the coated granules.

### Example 2

1 kilogram of coated granules was fabricated as follows.

304 grams of topiramate free acid, 566 grams of LUTROL F127, and 30 grams of polyvinyl pyrrolidone 12PF (PVP) were passed through a #40 mesh sieve and dry mixed. Prior to this sizing operation, the drug particle size was nominally about 100 microns and the LUTROL F127 had been micronized to a finely divided size. 100 grams of POLYOX N80 was passed through a #50 mesh sieve and blended into the mixture. The mixed powders were introduced to a small bowl mixer. The mixer was started and while mixing the powders, 100 ml of anhydrous ethanol was slowly and continuously added to the mixture, forming a uniform, damp mass. The damp mass was removed from the mixer and spread on open trays to air dried overnight in a fume hood. The dried mass was then passed through a #20 mesh sizing sieve.

The resulting bulk granules were transferred to a mini-Glatt fluid bed granulator (FBG) and fluidized in a current of warm, drying air. A solution of wall-forming polymer was spray coated onto the granules. The solution consisted of 5 weight percent PVP 12PF dissolved in water. The spraying continued until the granules accumulated a PVP wall coating weight of 24 percent. During the spraying process, the bed of granules was removed four times and passed each time through a #16 mesh sizing screen to break up agglomerates. The bed of granules was also sized through the 16-mesh sieve after the 24 weight percent had been coated.

The PVP-coated granules were returned to the FBG unit and spray coated with a second wall-forming composition. This solution consisted of 5 weight percent methylcellulose A15LV (MC) dissolved in water. This second solution was spray coated onto the granules and small samples of the granules were removed during the process at weight gains representing 1%, 2% and 3% MC. Each fraction was sized through a #16 screen. This produces coated granules with diameters of about 1.2 millimeters (47 mils).

These processes formed four batches of coated granules, one sample of granules coated with a single layer of PVP and three batches of granules coated with bilayers of PVP/MC.

Samples of the bare core granule (prior to any coating), granules with 24% PVP, granules with 24% PVP+1% MC, granules with 24% PVP+2% MC, and granules with 24% PVP+3% MC were assembled for comparative testing for their smear resistance. A small portion of each was placed on a hard flat surface at room temperature. Each sample was pressed and sheared manually with a spatula to test smearing. The resistance to smearing was observed as follows: bare granules <<0% MC<1% MC<2% MC<3% MC. The relatively thick layer of PVP provided a thick subcoat for more complete coverage of the granule core. The thin, cellulosic top coat provided improved resistance to crushing and smearing.

### Example 3

Solubilizing granules of nearly equivalent granule core and coating composition to the granules described in Example 2 were made at the 30 kg scale using pilot manufacturing scale equipment. The resulting coated granules were fed to a Korsch multiplayer press to assess smearing. After formulating these granules into tablets for about 30 minutes, the surface of the press turret was clean and almost completely free of smeared material.

Delivery systems were then hand fabricated with this batch of coated granules and tested for release of drug. The performance of these systems with coated granules at the 30 kg scale was compared to the performance of systems encapsulated at the 1 kg scale by measuring release rates.

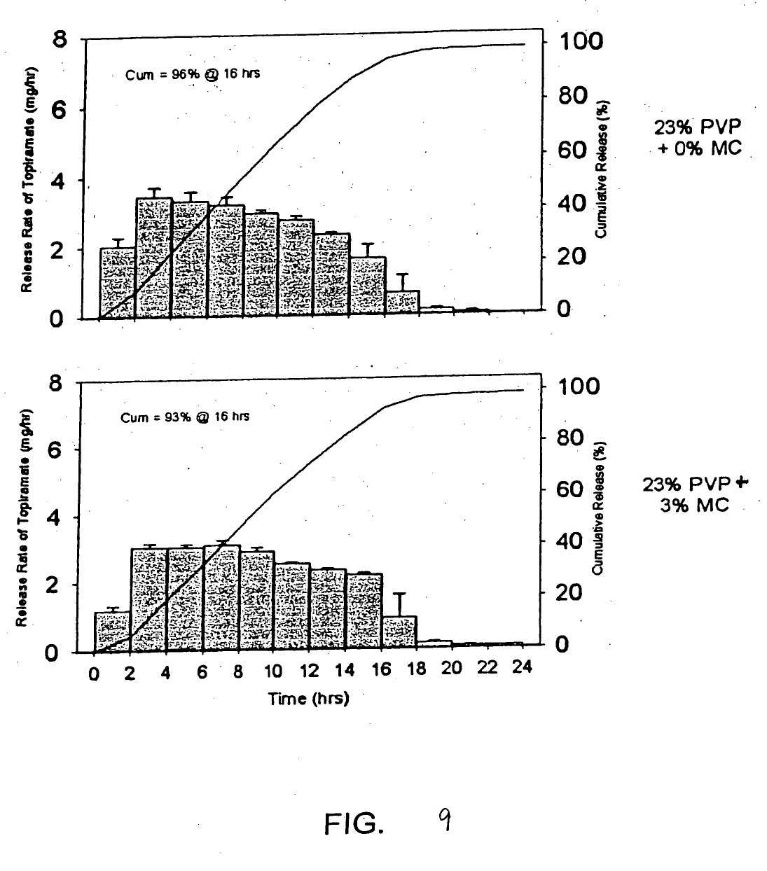

FIG. 8 shows topiramate release patterns of control release topiramate dosage forms having coated granules prepared in the 1 kg and 30 kg batch sizes. The results show that the release patterns of both production systems were equivalent. That is, no differences were found in the time to reach the maximum release rate, the magnitude of the maximum release rate, and the period in which the maximum release rate is maintained.

### Example 4

The coated granules of Example 3 were compressed manually into tablets with push layers. The drug layer weights were fixed at 182 mg and the push layer weight were fixed at 60 mg. Drug contents were about 45 mg. The resulting tablets were coated with rate controlling membranes (semi-permeable membranes), drilled with 40 mil ports, and dried.

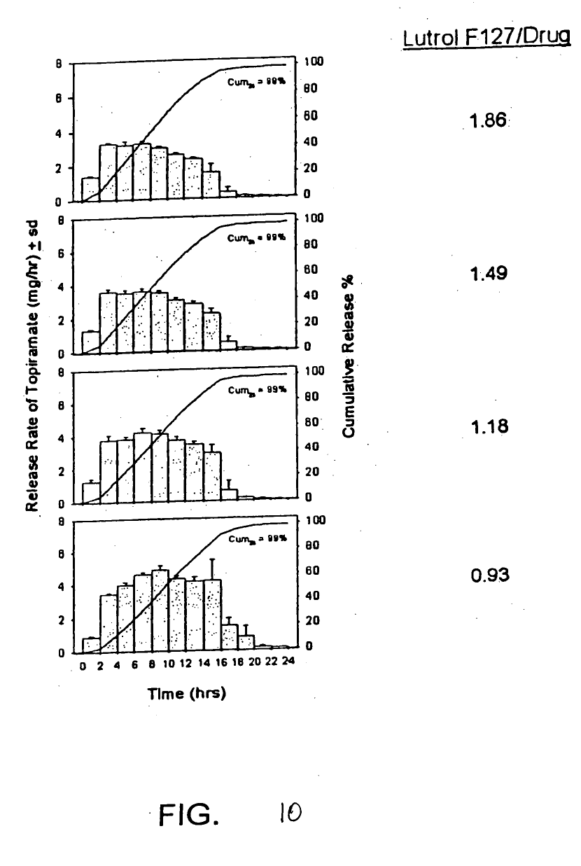

FIG. 9 shows how bilayer granule coatings can extend the period of zero order release kinetics. In all cases the first layer of the granule coating was 23% PVP. An outer, second layer containing MC was added. A comparison of release patterns obtained with 0% MC and 3% MC granule coating layer show that the bilayer granule coating can extend the period of zero order drug delivery. That is, the 3% MC second layer extended the zero order release rate by at least two hours.

### Example 5

A polyethylene oxide encapsulated drug formulation of the invention was prepared as follows.

First, an encapsulation solution was prepared. 4.5 kg of Polyethylene oxide N-10 (approximately 1 million molecular weight) was dissolved in 70.5 kg of water. Then, 26 kg of the drug granulation described in Example 4 was added to a fluid bed granulator bowl. The granulation was fluidized and mixed while 50 kg of coating solution was sprayed from a nozzle onto the powder. The coated granules were dried in the fluid-bed chamber to an acceptable moisture level. The coated granules were sized using a Fluid Air mill with a 7-mesh screen. The granulation was transferred to a Gemco rotational tumbler, mixed with 5.6 g of butylhydroxytoluene and lubricated with 565 g of stearic acid.

The use-test was performed using a Korsch multilayer press. The feasibility of the method was clearly demonstrated: no smearing of the drug granulation on the turret was observed over the 30 minute run time. Original (non-encapsulated) granulation used as a control (Example 1) showed considerable smearing in the first 5 minutes of compression at equivalent parameters (See FIG. 1).

### Example 6

Coated granules with cores comprising topiramate, LUTROL F127, POLYOX N80, and PVP were encapsulated at the 1 kg scale according to the procedures described in Example 2 with wall (semi-permeable wall) compositions and weights of 23% PVP inner coating layer and a 3% MC outer coating layer. Four batches of coated granules were encapsulated with this bilayer wall. Each batch represented a different ratio of solubilizing surfactant to drug. Delivery systems were fabricated by hand following the procedures in Example 2 and tested for release of drug.

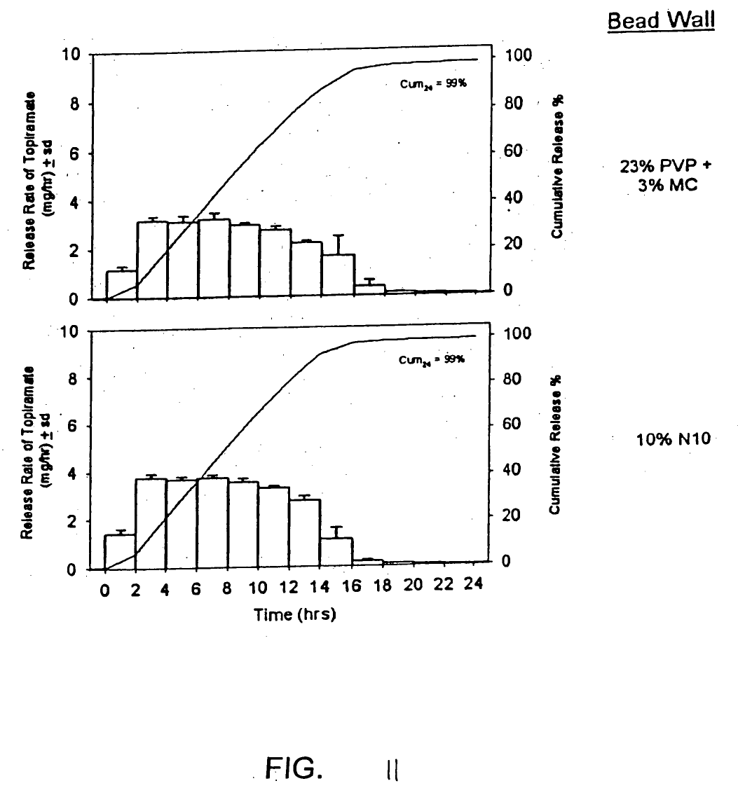

FIG. 10 represents the release profiles of the resulting systems. Drug release was 99% by 24 hours. System-to-system uniformity was excellent for LUTROL F127/drug ratios ranging from 1.86 to 1.18. Variability was observed at a Lutrol F127/Drug ratio=0.93. Therefore, the optimal ratio of topiramate/LUTROL F127 within the granules is in the range of about 1.9 to 1.2 inclusive. The lower the ratio, the greater the period of zero order release, the later the time to reach the maximum release rate and the greater the magnitude of the maximum release rate.

### Example 7

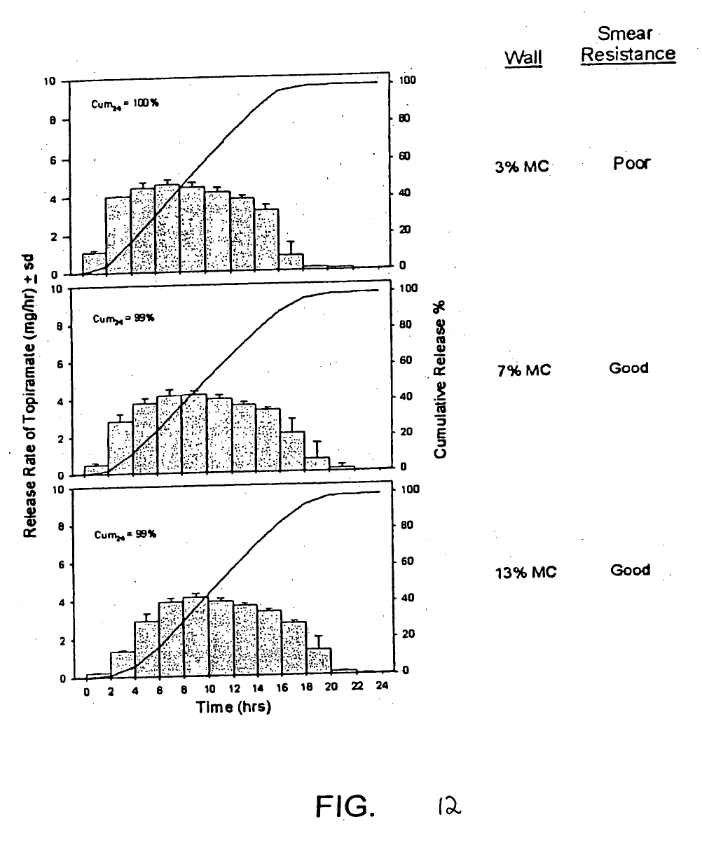

Changes in the granule coating can modulate the rate of drug delivery. FIG. 11 shows how changing bilayer coatings of topiramate granules can alter the delivery rate. Two compositions were prepared. Granules containing 28.8/53.6/9.58/1/0.02. topiramate/LUTROL F127/PVP K2932/POLYOX N80/stearic acid/Mg stearate/ BHT, were coated with 23% MC. The first was a bilayer consisting of a 23% PVP inner coating layer with a 3% MC outer coating layer. The second were the granules coated with a monolayer of 10% POLYOX N10. The results show a difference in the magnitude of the maximum release rate: the bilayer significantly lowered the maximum value of the release rate.

### Example 8

To test the effect that single-layer coating weight can have on smear resistance, a formulation was prepared from 35/52/10/3 topiramate/micronized LUTROL F127/POLYOX N80/PVP 12 PF. This mixture was coated to the target percentage weight with methylcellulose A15LV to form coated granules. These coated granules, 180 mg, were compressed with a 60 mg push layer (60 mg of 73.7/20/5/1/0.25, POLYOX303/NaCl/PVP K2932/Fe₂O₃/stearic acid/BHT). Smear resistance was assessed by force to eject tablets from die in manual compression mode.

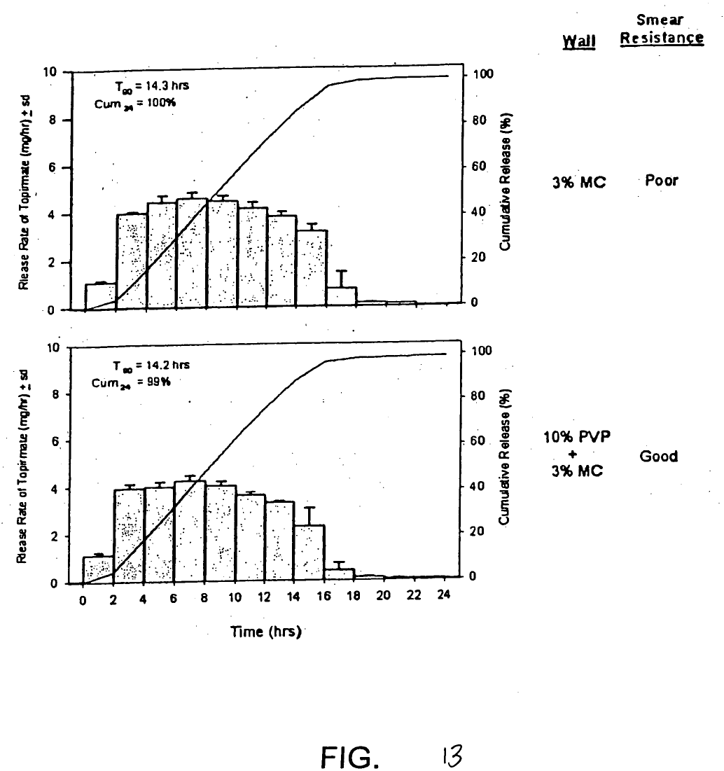

The results of tests are shown in FIG. 12. Resistance to smearing with 3% MC was poor, with 4% MC was fair and with 7% or greater MC, resistance was good. FIG. 12 shows dosage forms comprising 13% MC coated granules (a) have a period of zero order release kinetics that is extended by 2 hours compared to 3% MC; (b) have a delay of more than 2 hours in the time to reach maximum release rate compared to 3% MC.

### Example 9

To test the effect that bilayer coating can have on smear resistance, a formulation was prepared from 35/52/10/3 topiramate/micronized LUTROL F127/POLYOX N80/PVP 12 PF. This mixture was coated to the target percentage weight with PVP 12PF followed by methylcellulose A15LV to form coated granules. These coated granules, 180 mg, were compressed with a 60 mg push layer (60 mg of 73.7/20/5/1/0.25, POLYOX303/NaCl/PVP K2932/Fe₂O₃/stearic acid/BHT). The tablets were covered by a 1 mil smooth coat of 90/10 HEC/LUTROL F127, and two membrane—a membrane of 6 mils (1 mil=1/1000^{th} of an inch) thickness composed of of 55/40/5 EC100 cps/HPC EFX/MYRJ 52S and a membrane of 6 mils of 70/30 CA 398-1 0/Polaxamer 188, with a 1×40 mil orifice. Smear resistance was assessed by force to eject tablets from die in manual compression mode. Release rate was measured using USP II methods.

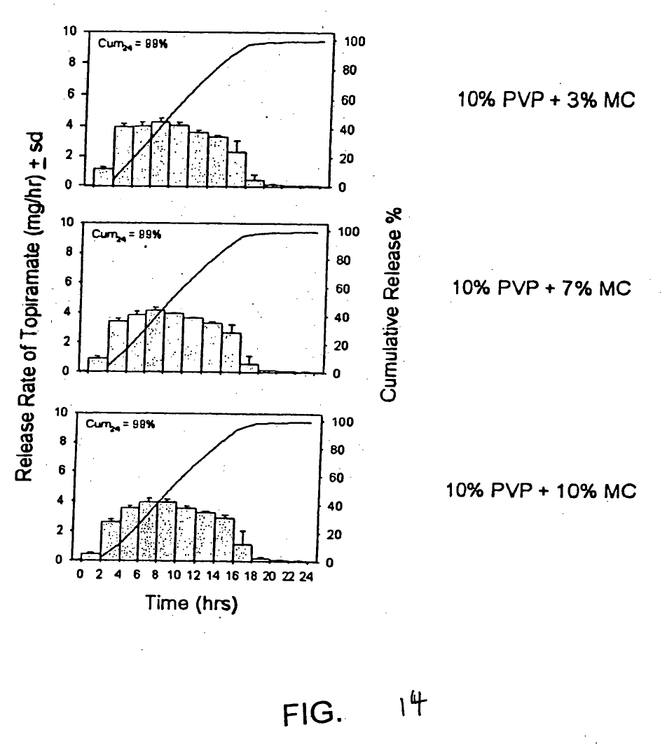

The results are shown in FIG. 13. The results show that the bilayer coating imparts smear resistance compared to single walls. The release patterns also show that smear resistance can be achieved without changing the release properties of the dosage form in an in vitro assay.

### Example 10

Granules were prepared as in Example 8 and coated with bilayers. The inner layer was 10% PVP and various weight percentages of MC were applied to the outer coating layer: 3%, 7% and 10%. FIG. 14 shows how changing single-layer coating weight modulates the time to reach a maximum delivery rate of topiramate. Increasing MC weight percentage in the outer coating layer increased the time to reach maximum release rate and decreased the period of zero order release rate.

### Example 11

### Bi-Layered Osmotic Dosage Form of Topiramate

A drug composition of the present invention was prepared as follows. Aqueous solutions of five surfactants were prepared. The selected surfactants were four grades of ethylene oxide/propylene oxide/ethylene oxide (LUTROL grades F127, F87, F 108, and F68) and PEG-40 stearate (MYRJ 52). Solutions were made at concentrations of 1, 5, and 15 weight percent. The aqueous surfactant blends solutions were chilled as necessary to promote complete dissolution of the surfactant prior to drug solubility studies. Each surfactant had a different HLB value and spanned a range of 16.9 to 29 HLB units.

The aqueous surfactant solutions were equilibrated to constant temperature in a 37° C. water bath. Then, neat topiramate drug was added slowly with stirring in about 10 mg increments to the surfactant solutions until no more drug dissolved. A control sample of drug dissolved in de-ionized water without surfactant was included for comparison purposes. The resulting saturated solutions of drug were filtered through 0.8 micron filters and analyzed for drug concentration by refractive index chromatography. The resulting solubility values were plotted as a function of both surfactant concentration and the hydrophilic-lipophilic balance value of each surfactant.

This method revealed three insights. Topiramate solubility in water was increased by each surfactant. Drug solubility was higher in the presence of each surfactant compared to the control where the solubility in de-ionized water without surfactant was 13.0 mg/ml. Second, a high concentration of surfactant was more effective in solubilizing drug than a low concentration. Third, the HLB values most effective to increase solubility of this drug were at the lower end, in the range of 16.9 to 22. The three concentrations of surfactant each formed the maximum solubility of topiramate with an HLB encompassing this range of HLB values.

Following this finding, a drug composition of the present invention was prepared. First, 55 grams of topiramate, 30 grams of granular LUTROL F 127, 11.5 grams of the polyethylene oxide (PEO) N80, and 3 grams of polyvinyl pyrrolidone (PVP) 2932 were passed through a #40 mesh sieve and the composition was dry mixed to a uniform blend wherein the PVP acts as a binder and the PEO acts as the structural polymer (carrier). The molecular weight of the polyethylene oxide was 200,000 grams per mole and the molecular weight of the polyvinyl pyrrolidone was approximately 10,000. The polyoxyethylene oxide serves as carrier and structural polymer 32. The polyvinyl pyrrolidone serves as the drug layer binder 36. The dry mixture was then wetted with anhydrous ethyl alcohol SDA 3A anhydrous and stirred to form a uniformly wetted mass. The wet mass was then passed through a 20-mesh sieve, forming damp noodles. The noodles were air dried at ambient conditions overnight, then passed again through a #20 mesh sieve, forming free-flowing granules. Finally, 0.5 grams of drug layer lubricant 34 magnesium stearate was passed through a # 60 mesh sieve over the granules and tumble mixed into the granules. This formed the drug composition granulation.

A push layer granulation was prepared in a similar manner. First, 89 grams of polyethylene oxide 303, 7 grams of sodium chloride, and 3 grams of hydroxypropyl methylcellulose E5 were passed through a #40 mesh sieve and dry mixed. The polyethylene oxide had a molecular weight of approximately 7,000,000 and the hydroxypropyl methylcellulose had a molecular weight of approximately 11,300. The polyethylene oxide served as the push layer osmopolymer 41 and the hydroxypropyl methylcellulose provided the push layer binder 43. Next, the dry mixture was wetted with anhydrous ethyl alcohol SDA 3A and mixed to a uniform damp mass. The mass was passed through a #20 mesh sieve forming noodles that were air dried overnight. Next, the noodles were passed again through a #20 mesh sieve forming free-flowing granules. Finally, 0.5 grams of minus #60 mesh magnesium stearate, push layer lubricant 44, was tumbled into the blend. This formed the push layer granulation.

A portion of the drug composition granulation weighing 182 mg was filled into a 3/16 inch diameter die cavity and lightly tamped with 3/16 inch biconvex round tablet tooling. Then, 60 mg of the push layer granulation was filled into the die and compressed and laminated to the drug layer using a force of 0.5 tons with a Carver press. Six of these bi-layer tablets were compressed.

Next, the tablets were coated with three layers. First, a solution was prepared by dissolving 57 grams of hydroxyethyl cellulose 250 L and 3 grams of polyethylene glycol in 940 grams of de-ionized water. The hydroxyethyl cellulose had a molecular weight of approximately 90,000 and the polyethylene glycol had a molecular weight of 3,350. This formed a smoothing coat solution to provide a smooth coatable surface for subsequent coatings.

The six active tablets were mixed into a tablet bed of placebo tablets that weighed 0.5 kg. The tablet bed was coated with the smoothing coat solution in an Aeromatic coater. The solution was applied in a current of warm, dry air until about 4 mg of coating weight was accumulated on each active tablet. The coating solution was stirred continuously during the coating process. The resulting smoothing coat produced a smooth tablet substrate and rounded the corners of the tablets. The resulting smooth tablets were dried in a 40° C. force air oven overnight. (This smoothing coat is optional and is especially useful to round the corners of the tablets where tablet lands have flash from the compression process.)

The next coating solution was prepared by dissolving 269.5 grams of ethyl cellulose 100 cps, 196.0 grams of hydroxypropyl cellulose EFX, and 24.5 grams of MYRJ 52 in 6510 grams of anhydrous ethanol SDA3A with stirring and warming. The ethyl cellulose had a molecular weight of approximately 220,000 and the hydroxypropyl cellulose had a molecular weight of approximately 80,000. The solution was allowed to stand at ambient temperature. This formed the membrane subcoat solution.

The smooth tablets from above were mixed into a bed of placebo tablets weighing 1.2 kg and the resulting mixed bed was charged into a Vector LDCS pan coater fitted with a 14 inch diameter coating pan. The membrane subcoat solution was then sprayed onto the bed of tablets in the coater in a current of warm air. The coating solution was stirred continuously during the process. The solution was applied in this manner until about 5.5 mils of coating was accumulated on each drug tablet.

Then, 175 grams of cellulose acetate 398-10 and 75 grams of LUTROL F68 were dissolved in 4,750 grams of acetone with warming and stirring. The cellulose acetate had an average acetyl content of about 39.8 weight percent and a molecular weight of approximately 40,000. This formed the membrane overcoat solution.

This membrane overcoat solution was applied to the bed of active and placebo cores in the LDCS pan coater until 5 mils of membrane overcoat accumulated on each drug tablet. The three-coated layers formed wall 20 of the present invention. An exit orifice 60 was mechanically drilled through the three coating layers on the drug layer side of the tablets using a 40 mil diameter drill bit and drill press. The systems were then dried in a forced air oven at 40° C. to remove residual processing solvents.

The resulting six dosage forms (systems) were tested for release of drug as a function of time in de-ionized water at 37° C. by sampling every 2 hours over a duration of 24 hours. Drug release was monitored with refractive index chromatography. The drug 31 was delivered at an ascending release pattern for 12-14 hours. The time to deliver 90% of the 100 mg dose was about 18 hours. The cumulative delivery at 24 hours was 97.5%. The membranes were intact throughout the delivery pattern.

The dosage forms were sufficiently small to easily be swallowed by a patient even with the high drug loading of 55% present in the drug composition 30.

Similar dosage forms with push layers were formulated with 55% drug in the drug composition, but without the solubilizing surfactant in an attempt to implement prior art technology. These dosage forms of the prior art were not operational. The drug compositions representing the prior art did not solublize the drug and resulted in drug compositions that could not be pumped from the dosage forms. The membranes of these dosage forms split open in situ during in vitro testing, dumping the bolus of drug in an uncontrolled fashion. The splitting of the dosage forms was due to the strain induced within the membrane by the swelling pressure generated by the push layer pushing against the insoluble drug composition through the narrow 40 mil port.

### Example 12

### Bi-Layered Topiramate Dosage Form

A drug composition of 9.0 grams of micronized LUTROL F 127 was dry mixed with 16.5 grams of topiramate. The topiramate had a nominal particle size of 80 microns. Next, 3.45 grams POLYOX N80 and 0.9 grams of polyvinyl pyrrolidone were sieved through a minus 40 mesh and blended into the mixture. Then, 5 grams of anhydrous ethanol was added slowly with stirring to form a damp mass. The damp mass was passed through a #16 mesh sieve and air dried overnight at ambient temperature. The resulting dried noodles were passed again through #16 mesh sieve. Then, 150 mg of magnesium stearate was passed through a #60 mesh sieve over the dried granules and tumble mixed into the granules. The concentration of surfactant in this drug composition granulation was 30 weight percent.

The push layer granulation was prepared by passing 63.67 grams of POLYOX 303, 30 grams of sodium chloride, and 5 grams of hydroxypropyl methylcellulose through a #40 mesh sieve and dry mixing to form a uniform blend. Then, 1.0 gram of ferric oxide red was passed though a #60 mesh sieve into the mixture. The resulting mixture was wet massed by slowly adding anhydrous ethyl alcohol SDA3A anhydrous with stirring to form a uniformly damp mass. The mass was passed through a # 20 mesh sieve, resulting in noodles that were dried at 40° C. in forced air overnight. The dried noodles were passed through a # 16 mesh sieve to form free-flowing granules. Finally, 25 mg of magnesium stearate and 8 mg of butylated hydroxytoluene were sieved through a # 80 mesh sieve into the granules and tumble mixed.

A portion of the drug composition granulation weighing 182 mg was filled into a round 3/16-inch diameter die and lightly compressed with 3/16-inch concave punches. Then, 60 mg of the push layer granulation was added to the drug layer and the two layers were laminated with a force of 800 pounds. Six tablets were made.

The tablets were coated as described in Example 11 with 5 mg of the smoothing coat, 5.4 mils of the subcoat membrane, and 5.7 mils of the overcoat membrane. One exit port of 40 mils diameter was drilled through the three coating layers and the systems were dried overnight at 40° C. in forced air.

The resulting dosage forms were tested as described in Example 11. The systems released 99% of the drug over a 24 hour duration. The release rate was substantially ascending during the first 14 hours over which time about 76% of the drug was released. The system released about 90% of the drug over 19 hours. The final system was of the same size that is convenient and feasible for patients in need to swallow as described in Example 11.

### Example 13

### Bi-Layered Topiramate Dosage Forms

Systems were made as described in Example 12 except that the surfactant 33 comprised a blend of two solubilizing surfactants. The drug composition granulation was made according to the procedure in Example 2 except that the surfactant consisted of 15 weight percent micronized LUTROL F127 and 15 weight percent MYRJ 52 substituted for the 30 weight percent micronized LUTROL F127. The weighted average HLB value of the two surfactants yielded an HLB value of 19.5, that is mid point between the two HLB values of the single surfactants.

The dosage forms delivered at a substantially zero order rate between hour 2 and hour 14. The dosage forms released 89% of the dose over 24 hours.

### Example 14

### Bi-Layered Topiramate Dosage Forms

Dosage forms were made as described in Example 13 but with a larger weight of the push layer. The push layer weight was 90 mg substituted for the 60 mg weight of the systems in Example 13.

The system delivered at a substantially ascending release rate for about 12 hours. After 12 hours, the rate became descending. The amount of drug delivered over 24 hours was about 93%.

### Example 15

### Bi-Layered Topiramate Dosage Form

A drug composition 30 was formed consisting of 30 wt. % drug topiramate, 56 wt % surfactant LUTROL F127, 10 wt % carrier POLYOX N80 and 3 wt% PVP K2932 and 2 wt % stearic acid by wet granulating with anhydrous ethanol.

A push layer consisting of 63.37 wt % POLYOX 303 (7,000,000 molecular weight), 30 wt % NaCl, 5 wt% HPMC E5, 1 wt % Ferric Oxide, 0.5 wt % Mg Stearate and 0.08 wt % BHT was wet granulated with anhydrous ethanol.

Tablets with 333 mg of the drug composition (100 mg topiramate) and 133 mg push layer were compressed using a 9/32″ longitudinally compressed tablet tooling. Total tablet (capsule shape) weight was 466 mg. The systems were coated, drilled, and dried according to the procedures described in Example 11. The systems were then tested for release of drug, producing a substantially zero order release pattern, delivering the drug at a steady rate of about 5.8 mg per hour over about 16 hours.

### Example 16

### Topiramate Capsule Shaped Tri-layer 100 mg System

A first drug composition was prepared as follows. First, 3000 g of topiramate, 2520 g of polyethylene oxide with average molecular weight of 200,000 and 3630 g of poloxamer 407 (LUTROL F127) having an average molecular weight of 12,000 were added to a fluid bed granulator bowl. Next two separate binder solutions, a poloxamer binder solution and a polyvinylpyrrolidone identified as K29-32 having an average molecular weight of 40,000 binder solution were prepared by dissolving 540 g of the same poloxamer 407 (LUTROL F127) in 4860 g of water and 495 g of the same polyvinylpyrrolidone in 2805 of water, respectively. The dry materials were fluid bed granulated by first spraying with 2700 g of the poloxamer binder solution and followed by spraying 2000 g of the polyvinylpyrrolidone binder solution. Next, the wet granulation was dried in the granulator to an acceptable moisture content 0.3%, and sized using by passing through a 7-mesh screen. Next, the granulation was transferred to a blender and mixed with 5 g of butylated hydroxytoluene as an antioxidant and lubricated with 200 g of stearic acid and 75 g of magnesium stearate.

A second drug composition was prepared as follows. First, 4000 g of topiramate, 213 g of polyethylene oxide with average molecular weight of 200,000, 4840 g of poloxamer 407 (LUTROL F127) having an average molecular weight of 12,000 and 10 g of ferric oxide, black were added to a fluid bed granulator bowl. Next, two separate binder solutions, a poloxamer binder solution and a polyvinylpyrrolidone identified as K29-32 having an average molecular weight of 40,000 binder solution were prepared by dissolving 720 g of the same poloxamer 407 in 6480 g of water and 495 g of the same polyvinylpyrrolidone in 2805 of water, respectively. The dry materials were fluid bed granulated by first spraying with 3600 g of the poloxamer binder solution and followed by spraying 2000 g of the polyvinylpyrrolidone binder solution. Next, the wet granulation was dried in the granulator to an acceptable moisture content, and sized by passing through a 7-mesh screen. Next, the granulation was transferred to a blender and mixed with 2 g of butylated hydroxytoluene as an antioxidant and lubricated with 200 g of stearic acid and 75 g of magnesium stearate.

Next, a push clayer was prepared as follows. First, a binder solution was prepared. 7.5 kg of polyvinylpyrrolidone identified as K29-32 having an average molecular weight of 40,000 was dissolved in 50.2 kg of water. Then, 37.5 kg of sodium chloride and 0.5 kg of ferric oxide were sized using a Quadro Comil with a 21-mesh screen. Then, the screened materials and 80.4 kg of polyethylene oxide (approximately 7,000,000 molecular weight) were added to a fluid bed granulator bowl. The dry materials were fluidized and mixed while 48.1 kg of binder solution was sprayed from 3 nozzles onto the powder. The granulation was dried in the fluid-bed chamber to an acceptable moisture level, 0.5%. The coated granules were sized using a Fluid Air mill with a 7-mesh screen. The granulation was transferred to a tote tumbler, mixed with 63 g of butylated hydroxytoluene and lubricated with 310 g stearic acid.

Next, the first and second drug compositions and the push layer were compressed into tri-layer tablets on multilayer Korsch press. First, 120 mg of the first drug composition was added to the die cavity and pre-compressed, then, 160 mg of the second drug composition was added to the die cavity and pre-compressed again, and finally, the push layer was added to achieve the total system weight of 480 mg and the layers were pressed into a ¼″ diameter, capsule shaped, deep concave, tri-layer arrangement.

The tri-layer arrangements were coated with bi-layer polymer membrane laminate in which the first coating layer was a rigid yet water permeable laminate and the second coating layer was a semi-permeable membrane laminate. The first membrane laminate composition comprised 55% ethylcellulose, 45% hydroxylpropyl cellulose and 5% POLYOXYL.40 stearate (PEG 40 stearate or MYRJ 52S). The membrane-forming composition was dissolved in 100% ethyl alcohol to make a 7% solids solution. The membrane-forming composition was sprayed onto and around the tri-layer arrangements in a 10 kg scale pan coater until about 45 mg of membrane was applied to each tablet.

Next, the tri-layer arrangements coated with the first membrane laminate were coated with the semi-permeable membrane. The membrane forming composition comprised 80% cellulose acetate having a 39.8% acetyl content and 20% poloxamer 188 (PLURONIC F68 or LUTROL F68). The membrane-forming composition was dissolved in 100% acetone solvent to make a 5% solids solution. The membrane-forming composition was sprayed onto and around the tri-layer arrangements in a pan coater until about 35 mg of membrane was applied to each tablet.

Next, one 40 mil (1 mm) exit passageway was laser drilled through the bi-layer membrane laminate to connect the drug layer with the exterior of the dosage system. The residual solvent was removed by drying for 72 hours at 40° C. and ambient humidity.

Next, the drilled and dried systems were color overcoated. The color overcoat was a 12% solids suspension of OPADRY in water. The color overcoat suspension was sprayed onto the tri-layer systems until an average wet coated weight of about 25 mg per system was achieved.

Next, the color-overcoated systems were clear coated. The clear coat was a 5% solids solution of OPADRY in water. The clear coat solution was sprayed onto the color coated cores until an average wet coated weight of about 10 mg per system was achieved.

The dosage form produced by this manufacture were designed to deliver 100 mg of topiramate in a substantially ascending rate of release at certain controlled-delivery rate from the core containing the first drug composition of 30% topiramate, 25.2% polyethylene oxide possessing a 200,000 molecular weight, 39% poloxamer 407 (LUTROL F127), 3% polyvinylpyrrolidone possessing a 40,000 molecular weight, 0.05% butylated hydroxytoluene, 2% stearic acid and 0.75% magnesium stearate, and the second drug composition of 40% topiramate, 2.13% polyethylene oxide possessing a 200,000 molecular weight, 52% poloxamer 407 (LUTROL F127), 3% polyvinylpyrrolidone possessing a 40,000 molecular weight, 0.1% black ferric oxide, 0.05% butylated hydroxytoluene, 2% stearic acid and 0.75% magnesium stearate. The push layer was comprised 64.3% polyethylene oxide comprising a 7,000,000 molecular weight, 30% sodium chloride, 5% polyvinylpyrrolidone possessing an average molecular weight of 40,000, 0.4% ferric oxide, 0.05% butylated hydroxytoluene (BHT), and 0.25% stearic acid. The bi-layer membrane laminate in which the first membrane layer was comprised of 55% ethylcellulose, 45% hydroxylpropyl cellulose and 5% POLYOXYL 40 stearate (PEG 40 stearate or MYRJ 52S), and the second membrane laminate was a semi-permeable wall which was comprised of 80% cellulose acetate of 39.8% acetyl content and 20% poloxamer 188 (PLURONIC F68 or LUTROL F68). The dosage form comprised one passageway, 40 mils (1 mm) on the center of the drug side. The final dosage form contained a color overcoat and a clear overcoat.

The final dosage forms released such that about 90% of the drug was release with a substantially ascending rate of release over about 16 hours.

### Example 17

### Topiramate Capsule Shaped Tri-layer 12.5 mg System

A dosage form was manufactured as follows beginning with the first drug composition. First, 4 g of topiramate, 40 g of polyethylene oxide with average molecular weight of 200,000, 4 g of poloxamer 407 (LUTROL F127) having an average molecular weight of 12,000 and 1.5 g of polyvinylpyrrolidone identified as K29-32 having an average molecular weight of 40,000 were added to a beaker or mixing bowl. Next, the dry materials were mixed for 60 seconds. Then 16 mL of denatured anhydrous alcohol was slowly added to blended materials with continuous mixing for about 2 minutes. Next, the freshly prepared wet granulation was allowed to dry at room temperature for about 16 hours, and passed through a 16-mesh screen. Next, the granulation were transferred to an appropriate container, mixed and lubricated with 0.5 g of stearic acid.

Next, the second drug composition was prepared as follows: 6 g of topiramate, 35.95 g of polyethylene oxide with average molecular weight of 200,000, 6 g of poloxamer 407 (LUTROL F127) having an average molecular weight of 12,000, 1.5 g of polyvinylpyrrolidone identified as K29-32 having an average molecular weight of 40,000 and 0.05 g of ferric oxide were added to a beaker or mixing bowl. Next, the dry materials were mixed for 60 seconds. Then 16 mL of denatured anhydrous alcohol was slowly added to blended materials with continuous mixing for about 2 minutes. Next, the freshly prepared wet granulation was allowed to dry at room temperature for about 16 hours, and passed through a 16-mesh screen. Next, the granulation were transferred to an appropriate container, mixed and lubricated with 0.5 g of stearic acid.

Next, a push layer was prepared as follows. First, a binder solution was prepared. 7.5 kg of polyvinylpyrrolidone identified as K29-32 having an average molecular weight of 40,000 was dissolved in 50.2 kg of water. Then, 37.5 kg of sodium chloride and 0.5 kg of ferric oxide were sized using a Quadro Comil with a 21-mesh screen. Then, the screened materials and 80.4 kg of polyethylene oxide (approximately 7,000,000 molecular weight) were added to a fluid bed granulator bowl. The dry materials were fluidized and mixed while 48.1 kg of binder solution was sprayed from 3 nozzles onto the powder. The granulation was dried in the fluid-bed chamber to an acceptable moisture level, 0.5%. The coated granules were sized using a Fluid Air mill with a 7-mesh screen. The granulation was transferred to a tote tumbler, mixed with 63 g of butylated hydroxytoluene and lubricated with 310 g stearic acid.

Next, the first and second drug compositions and the push layer were compressed into tri-layer tablets on the Carver Tablet Press. First, 56 mg of the first drug composition was added to the die cavity and pre-compressed, then, 67 mg of the second drug composition was added to the die cavity and pre-compressed again, and finally, the push layer was added to achieve the total system weight of 211 mg and the layers were pressed into a 3/16″ diameter capsule, deep concave, tri-layer arrangement.

The tri-layer arrangements were coated with bi-layer polymer membrane laminate in which the first coating layer was a rigid yet water permeable laminate and the second coating layer was a semi-permeable membrane laminate. The coating was performed on a 10 kg scale pan coater by spike-loading the topiramate tri-layer systems with the placebo tablets. The first membrane laminate composition comprised 55% ethylcellulose, 45% hydroxylpropyl cellulose and 5% POLYOXYL 40 stearate (PEG 40 stearate or MYRJ 52S). The membrane-forming composition was dissolved in 100% ethyl alcohol to make a 7% solids solution. The membrane-forming composition was sprayed onto and around the tri-layer arrangements in a pan coater until about 30 mg of membrane was applied to each tablet.

Next, the tr-ilayer arrangements coated with the first membrane laminate were coated with the semi-permeable membrane. The membrane forming composition comprised 80% cellulose acetate having a 39.8% acetyl content and 20% poloxamer 188 (PLURONIC F68 or LUTROL F68). The membrane-forming composition was dissolved in 100% acetone solvent to make a 5% solids solution. The membrane-forming composition was sprayed onto and around the tri-layer arrangements in a pan coater until about 25 mg of membrane was applied to each tablet.

Next, one 30 mil (0.76 mm) exit passageway was laser drilled through the bi-layer membrane laminate to connect the drug layer with the exterior of the dosage system. The residual solvent was removed by drying for 72 hours at 40° C. and ambient humidity.

Next, the drilled and dried systems were color overcoated. The color overcoat was a 12% solids suspension of OPADRY in water. The color overcoat suspension was sprayed onto the tri-layer systems until an average wet coated weight of about 15 mg per system was achieved.

The dosage form produced by this manufacture was designed to deliver 12.5 mg of topiramate in a substantially ascending rate of release at certain controlled-delivery rate from the core containing the first drug composition of 8% topiramate, 80% polyethylene oxide possessing a 200,000 molecular weight, 8% poloxamer 407 (LUTROL F127), 3% polyvinylpyrrolidone possessing a 40,000 molecular weight and 1% stearic acid, and the second drug composition of 12% topiramate, 71.9% polyethylene oxide possessing a 200,000 molecular weight, 12% poloxamer 407 (LUTROL F127), 3% polyvinylpyrrolidone possessing a 40,000 molecular weight, 0.1% ferric oxide and 1% stearic acid. The push layer was comprised of 64.3% polyethylene oxide comprising a 7,000,000 molecular weight, 30% sodium chloride, 5% polyvinylpyrrolidone possessing an average molecular weight of 40,000, 0.4% ferric oxide, 0.05% butylated hydroxytoluene (BHT), and 0.25% stearic acid. The bi-layer membrane laminate in which the first membrane layer was comprised of 55% ethylcellulose, 45% hydroxylpropyl cellulose and 5% POLYOXYL 40 stearate (PEG 40 stearate or MYRJ 52S), and the second membrane laminate was a semi-permeable wall which was comprised of 80% cellulose acetate of 39.8% acetyl content and 20% poloxamer 188 (PLURONIC F68 or LUTROL F68). The dosage form comprised one passageway, 30 mils (0.76 mm) on the center of the drug side. The final dosage form could contained a color overcoat and a clear overcoat.

The final dosage form release topiramate such that about 90% of the drug was release with a substantially ascending rate of release over about 16 hours.

### Example 18

### Topiramate Capsule Shaped Bi-layer 100 mg System

A dosage form was manufactured as follows. First, 2880 g of topiramate, 958 g of polyethylene oxide with average molecular weight of 200,000 and 4980 g of poloxamer 407 (LUTROL F127) having an average molecular weight of 12,000 were added to a fluid bed granulator bowl. Next two separate binder solutions, a poloxamer binder solution and a polyvinylpyrrolidone identified as K29-32 having an average molecular weight of 40,000 binder solution were prepared by dissolving 500 g of the same poloxamer 407 (LUTROL F127) in 4500 g of water and 750 g of the same polyvinylpyrrolidone in 4250 of water, respectively. The dry materials were fluid bed granulated by first spraying with 3780 g of the poloxamer binder solution and followed by spraying 3333 g of the polyvinylpyrrolidone binder solution. Next, the wet granulation was dried in the granulator to an acceptable moisture content, 0.5%, and sized using by passing through a 7-mesh screen. Next, the granulation was transferred to a blender and mixed with 2 g of butylated hydroxytoluene (BHT) as an antioxidant and lubricated with 200 g of stearic acid and 100 g of magnesium stearate.

Next, a push layer was prepared as follows. First, a binder solution was prepared. 7.5 kg of polyvinylpyrrolidone identified as K29-32 having an average molecular weight of 40,000 was dissolved in 50.2 kg of water. Then, 37.5. kg of sodium chloride and 0.5 kg of ferric oxide were sized using a Quadro Comil with a 21-mesh screen. Then, the screened materials and 80.4 kg of polyethylene oxide (approximately 7,000,000 molecular weight) were added to a fluid bed granulator bowl. The dry materials were fluidized and mixed while 48.1 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 63 g of butylated hydroxytoluene and lubricated with 310 g stearic acid.

Next, the drug composition and the push composition were compressed into bi-layer tablets on multilayer Korsch press. First, 278 mg of the drug composition was added to the die cavity and pre-compressed, then, the push composition was added to achieve the total system weight of 463 mg and the layers were pressed into a 15/64″ diameter, capsule shaped, deep concave, bi-layer arrangement.

The bi-layer arrangements were coated with bi-layer polymer membrane laminate in which the first coating layer was a rigid yet water permeable laminate and the second coating layer was a semi-permeable membrane laminate. The first membrane laminate composition comprised 55% ethylcellulose, 45% hydroxylpropyl cellulose and 5% POLYOXYL 40 stearate (PEG 40 stearate or MYRJ 52S). The membrane-forming composition was dissolved in 100% ethyl alcohol to make a 7% solids solution. The membrane-forming composition was sprayed onto and around the arrangements in a pan coater until about 38 mg of membrane was applied to each tablet.

Next, the bi-layer arrangements coated with the first membrane laminate were coated with the semi-permeable membrane. The membrane forming composition comprised 80% cellulose acetate having a 39.8% acetyl content and 20% poloxamer 188 (PLURONIC F68 or LUTROL F68). The membrane-forming composition was dissolved in 100% acetone solvent to make a 5% solids solution. The membrane-forming composition was sprayed onto and around the arrangements in a pan coater until about 30 mg of membrane was applied to each tablet.

Next, one 45 mil (1.14 mm) exit passageway was laser drilled through the bi-layer membrane laminate to connect the drug layer with the exterior of the dosage system. The residual solvent was removed by drying for 72 hours at 40° C. and ambient humidity.

Next, the drilled and dried dosage forms were coated with an immediate release drug overcoat. The drug overcoat was a 13% solids aqueous solution containing 780 g of topiramate, 312 g of coPOVIDONE (KOLLIDONE VA 64) and 208 g of hydroxypropyl methycellulose possessing an average molecular weight of 11,200. The drug overcoat solution as sprayed onto the dried coated cores until an average wet coated weight of about 33 mg per system was achieved.

Next, the drug-over coated systems were color over coated. The color overcoat was a 12% solids suspension of OPADRY in water. The color overcoat suspension was sprayed onto the drug over coated systems until an average wet coated weight of about 25 mg per system was achieved.

Next, the color-over coated systems were clear coated. The clear coat was a 5% solids solution of OPADRY in water. The clear coat solution as sprayed onto the color coated cores until an average wet coated weight of about 25 mg per system was achieved.

The dosage form produced by this manufacture was designed to deliver 20 mg of topiramate as an immediate release from an overcoat comprised of 60% topiramate, 24% co-POVIDONE and 16% hydroxypropyl methylcellulose followed by the controlled delivery of 80 mg of topiramate from the drug composition containing 28.8% topiramate, 9.58% polyethylene oxide possessing a 200,000 molecular weight, 53.6% poloxamer 407 (LUTROL F127), 5% polyvinylpyrrolidone possessing a 40,000 molecular weight, 0.02% butylated hydroxytoluene (BHT), 2% stearic acid and 1% magnesium Stearate. The push layer was comprised 64.3% polyethylene oxide comprising a 7,000,000 molecular weight, 30% sodium chloride, 5% polyvinylpyrrolidone possessing an average molecular weight of 40,000, 0.4% ferric oxide, 0.05% butylated hydroxytoluene, and 0.25% stearic acid. The bi-layer membrane laminate in which the first membrane layer was comprised of 55% ethylcellulose, 45% hydroxylpropyl cellulose and 5% POLYOXYL 40 stearate (PEG 40 stearate or MYRJ 52S), and the second membrane laminate is a semi-permeable wall which was comprised of 80% cellulose acetate of 39.8% acetyl content and 20% poloxamer 188 (PLURONIC F68 or LUTROL F68). The dosage form comprised one passageway, 45 mils (1.14 mm) on the center of the drug side. The final dosage form contained a color overcoat and a clear overcoat.

The final dosage form had a mean release rate of 6 mg topiramate per hour releasing the topiramate with a substantially zero-order rate or release.

### Examples 19-24

### Topiramate Dosage Forms

Tables 1-9 below list composition details for additional embodiments of the present invention. More particularly, the tables below provide details on the composition of tri-layer, controlled release, osmotic dosage forms containing topiramate. Said dosage forms comprised two drug compositions, wherein the amount and/or concentration of topiramate in the two drug compositions was different, and a push layer.

Each of the dosage forms described below was prepared according to the procedure described in Example 25, by selecting and substituting the suitable components.

Table 1 below lists the components of dosage forms as a function of total dosage of topiramate. For each layer or coating, weights are listed in milligrams (e.g for the drug layers, push layers, semi-permeable membranes, other coatings, etc.). Also listed in Table 1 are the sizes for each dosage form, as prepared.

TABLE 1
Dosage Form Components

| Dosage | 10 mg | 20 mg | 45 mg | 90 mg | 135 mg | 180 mg |
|---|---|---|---|---|---|---|
| Size (inches) | 3/16 | 15/64 | 3/16 | 15/64 | 17/64 | 9/32 |
| Drug Layer 1 | 60 | 120 | 60 | 120 | 180 | 240 |
| Drug Layer 2 | 60 | 120 | 60 | 120 | 180 | 240 |
| Push Layer | 90 | 180 | 90 | 180 | 270 | 360 |
| Subcoat | 15 | 20 | 15 | 20 | 25 | 25 |
| Membrane Coat (99:1 CA:poloxamer) | 32 | 40 | 36 | 40 | 46 | 50 |
| Membrane Coat (78:22 CAB:poloxamer) | 28 | 38 | 28 | 38 | 42 | 48 |

CA = cellulose acetate
CAB = cellulose acetate butyrate

Table 2 below lists the components and amounts used in the preparation of the first drug composition for dosage forms comprising 45-180 mg total of topiramate. Target % (wt/wt) in granulation is the weight percent of the component as a function of the total weight of the drug layer.

TABLE 2
First Drug Composition (45-180 mg Dosages)

| | Target % (wt/wt) |
|---|---|
| Material ID | in Granulation |
| Topiramate | 32.00 |
| Polyethylene Oxide, NF, N-80, 200K, TG, LEO | 16.23 |
| POVIDONE, USP, Ph Eur, (K29-32) | 3.00 |
| Poloxamer 407, NF (Micronized) | 42.00 |
| Methylcellulose, USP, 15CPS, (A15-LV-PREMIUM) | 2.50 |
| Stearic Acid, NF, Ph Eur (Powder) | 3.00 |
| Magnesium Stearate, NF, Ph Eur | 1.25 |
| BHT, FCC, Ph Eur (Milled) | 0.02 |

Table 3 below lists the components and amounts used in the preparation of the second drug composition for dosage forms comprising 45-180 mg total of topiramate. Target % (wt/wt) in granulation is the weight percent of the component as a function of the total weight of the drug layer.

TABLE 3
Second Drug Composition (45-180 mg Dosages)

| | Target % (wt/wt) |
|---|---|
| Material ID | in Granulation |
| Topiramate | 43.00 |
| POVIDONE, USP, Ph Eur, (K29-32) | 3.00 |
| Poloxamer 407, NF (Micronized) | 49.90 |
| Methylcellulose, USP, 15CPS, (A15-LV-PREMIUM) | 2.50 |
| Ferric Oxide, NF, (Yellow) | 0.08 |
| Stearic Acid, NF, Ph Eur (Powder) | 1.00 |
| Magnesium Stearate, NF, Ph Eur | 0.50 |
| BHT, FCC, Ph Eur (Milled) | 0.02 |

Table 4 below lists the components and amounts used in the preparation of first drug composition for dosage forms comprising 10-20 mg total of topiramate. Target % (wt/wt) in granulation is the weight percent of the component as a function of the total weight of the drug layer.

TABLE 4
First Drug Composition (10-20 mg dosages)

| | Target % (wt/wt) |
|---|---|
| Material ID | in Granulation |
| Topiramate | 5.00 |
| Polyethylene Oxide, NF, N-80, 200K, TG, LEO | 88.73 |
| Poloxamer 407, NF (Micronized) | 2.00 |
| POVIDONE, USP, Ph Eur, (K29-32) | 3.00 |
| Stearic Acid, NF, Ph Eur (Powder) | 1.00 |
| Magnesium Stearate, NF, Ph Eur | 0.25 |
| BHT, FCC, Ph Eur (Milled) | 0.02 |

Table 5 below lists the components and amounts used in the preparation of the second drug composition for dosage forms comprising 10-20 mg total of topiramate. Target % (wt/wt) in granulation is the weight percent of the component as a function of the total weight of the drug layer.

TABLE 5
Second Drug Composition (10-20 mg Dosages)

| | Target % (wt/wt) |
|---|---|
| Material ID | in Granulation |
| Topiramate | 12.00 |
| Polyethylene Oxide, NF, N-80, 200K, TG, LEO | 71.72 |
| Poloxamer 407, NF (Micronized) | 12.00 |
| POVIDONE, USP, Ph Eur, (K29-32) | 3.00 |
| Iron Oxide, Red | 0.01 |
| Stearic Acid, NF, Ph Eur (Powder) | 1.00 |
| Magnesium Stearate, NF, Ph Eur | 0.25 |
| BHT, FCC, Ph Eur (Milled) | 0.02 |

Table 6 below lists the components and amounts used in the preparation of the push layer for all dosage forms topiramate. Target % (wt/wt) in granulation is the weight percent of the component as a function of the total weight of the drug layer.

TABLE 6
Push Layer Composition

| | Target % (wt/wt) |
|---|---|
| Material ID | in Granulation |
| Polyethylene Oxide, NF, 303, 7000K, TG, LEO | 64.3 |
| Sodium Chloride, USP, Ph Eur, (Powder) | 30.0 |
| POVIDONE, USP, Ph Eur, (K29-32) | 5.0 |
| Ferric Oxide, NF, (Red) | 0.1 |
| Ferric Oxide, NF, (Yellow) | 0.3 |
| Stearic Acid, NF, Ph Eur, (Powder) | 0.25 |
| BHT, FCC, Ph Eur, (Milled) | 0.05 |

Table 7 below lists the components and amounts used in the preparation of the subcoat (aqueous subcoat) for all dosage forms topiramate. Target % (wt/wt) in subcoat formulation is the weight percent of the component as a function of the total weight of the subcoat.

TABLE 7
Subcoat Composition

| | Target % (wt/wt) in |
|---|---|
| Material ID | Subcoat Formulation |
| Hydroxyethyl Cellulose, NF | 95 |
| Polyethylene Glycol 3350, NF, Ph Eur, LEO | 5 |

Tables 8 and 9 below list the components and amounts used in the preparation of the CAB (cellulose acetate butyrate) membrane coat and the CA (cellulose acetate) membrane coat, respectively, for all dosage forms of topiramate. Target % (wt/wt) in subcoat formulation is the weight percent of the component as a function of the total weight of the subcoat.

TABLE 8
CAB Membrane Coat

| | Target % (wt/wt) in |
|---|---|
| Material ID | Subcoat Formulation |
| Cellulose Acetate Butyrate (171-15) | 78 |
| Poloxamer 188, NF, Ph Eur | 22 |

TABLE 9
CA Membrane Coat

| | Target % (wt/wt) in |
|---|---|
| Material ID | Subcoat Formulation |
| Cellulose Acetate, NF, (398-10) | 99 |
| Poloxamer 188, NF, Ph Eur | 1 |

### Example 25

### Large Scale Manufacture of Topiramate Dosage Forms

A push layer granulation was manufactured as follows. The composition of the push layer was as follows: 64.3% polyethylene oxide, 30% sodium chloride, 5% POVIDONE, 0.4% ferric oxide, 0.25% stearic acid and 0.05% butylated hydroxytoluene.

A binder solution was prepared as follows: 7.5 kg of POVIDONE was added to 50.2 kg of purified water in a mixing vessel and mixed until the POVIDONE was completely in solution. The net weight of the prepared binder solution was determined by weighing.

The dry ingredients—80.4 kg of polyethylene oxide, 37.5 kg of sodium chloride and 0.5 kg of ferric oxide were charged into a tote. The fluid bed granulator was assembled with the guns required for spraying the binder solution. The granulator was then warmed to an inlet air temperature of 43-47° C. and 48 kg of the binder solution was metered into the granulator. After the spraying was completed, the granules were allowed to dry in the granulator until a moisture content less than or equal to 1% was obtained. The dried granules were then milled through a Granumill using a 7 mesh screen. The milled granulation was weighed and collected in a tote. 0.05% butylated hydroxytoluene by weight of the granulation was added to the tote and the granulation was mixed for 5 min. Stearic acid amount equivalent to 0.25% of the granulation was weighed and added to the tote. The granules were then mixed for an additional 5 minutes.

A granulation for the first drug composition was manufactured as follows. The composition of the first drug composition was as follows: 32% topiramate, 16.23% polyethylene oxide, 42% poloxamer407, 3% POVIDONE, 2.5% methylcellulose, 3% stearic acid, 1.25% magnesium stearate and 0.02% butylated hydroxytoluene.

A binder solution was prepared as follows: 480 g of POVIDONE was added to 4.32 kg of purified water in a mixing vessel and mixed until the POVIDONE was completely in solution. The net weight of the prepared binder solution was determined by weighing.

A methylcellulose granule coating solution was prepared as follows: 2.6 kg of purified water was heated to a temperature greater than 50° C. 400 g of methylcellulose is gradually added to the hot water while mixing. Mixing was continued until all solids were dispersed. 5 kg of purified water was then added to the mixing vessel and mixing was continued until all solids were dissolved. The net weight of prepared granule coating solution was determined by weighing.

The dry ingredients—3.2 kg topiramate, 1.623 kg polyethylene oxide, and 4.2 kg poloxamer were charged into a tote. The fluid bed granulator was assembled with the guns required for spraying the binder solution. The granulator was then warmed to an exhaust air temperature less than 25° C. and 3 kg of the binder solution was metered into the granulator. Following the spraying of the binder solution, 5 kg of granule coating solution was sprayed onto the granules. After spraying was completed, the granules were allowed to dry in the granulator until a moisture content less than or equal to 0.5% was obtained. The dried granulation was then milled through a Granumill using a 7 mesh screen. The milled granulation was weighed and collected in a tote. 0.05% butylated hydroxytoluene by weight of the granulation was added to the tote and the granulation was mixed for 5 min. Stearic acid amount equivalent to 3% of the granulation was weighed and added to the tote. The granules were then mixed for an additional 5 minutes. Magnesium stearate amount equivalent to 1.25% of the granulation was weighed and added to the tote. The granules were then mixed for an additional 30 seconds.

A granulation for the second drug composition was manufactured as follows. The composition of the second drug composition was as follows: 43% topiramate, 49.9% poloxamer 407, 3% POVIDONE, 2.5% methylcellulose, 1% stearic acid, 0.5% magnesium stearate, 0.08% yellow ferric oxide, and 0.02% butylated hydroxytoluene.

A binder solution was prepared as follows: 480 g of POVIDONE was added to 4.32 kg of purified water in a mixing vessel and mixed until the POVIDONE was completely in solution. The net weight of the prepared binder solution was determined by weighing.

A methylcellulose granule coating solution was prepared as follows: 2.6 kg of purified water was heated to a temperature greater than 50° C. 400 g of methylcellulose was gradually added to the hot water while mixing. Mixing was continued until all solids were dispersed. 5 kg of purified water was then added to the mixing vessel and mixing was continued until all solids are dissolved. The net weight of prepared granule coating solution was determined by weighing.

The dry ingredients—4.3 kg Topiramate, 4.9 kg poloxamer and 8 g ferric oxide were charged into a tote. The fluid bed granulator was assembled with the guns required for spraying the binder solution. The granulator was then warmed to an exhaust air temperature less than 25° C. and 3 kg of the binder solution was metered into the granulator. Following the spraying of the binder solution, 5 kg of granule coating solution was sprayed onto the granules. After spraying was completed, the granules were allowed to dry in the granulator until a moisture content less than or equal to 0.5% was obtained. The dried granulation was then milled through a Granumill using a 7 mesh screen. The milled granulation was weighed and collected in a tote. 0.05% butylated hydroxytoluene by weight of the granulation was added to the tote and the granulation was mixed for 5 min. Stearic acid amount equivalent to 1% of the granulation was weighed and added to the tote. The granules were then mixed for an additional 5 minutes. Magnesium stearate amount equivalent to 0.5% of the granulation was weighed and added to the tote. The granules were then mixed for an additional 30 seconds.

Compression of cores was completed as follows. The above granulations were compressed into a trilayer tablet core. Different weights were compressed into different size cores for the various doses.

A trilayer tablet core to deliver 90 mg drug was compressed as follows: 28.6% by weight of drug layer 1, 28.6% by weight of drug layer 2 and 42.9% by weight of push layer were compressed to form a trilayer tablet on a Korsch Tablet press. For the 90 mg tablet 120 mg drug layer 1, 120 mg drug layer 2 and 180 mg push layer were compressed together using a 15/64″ diameter tooling set.

Subcoat application was completed as follows. The composition of the subcoat was as follows: 95% hydroxyethyl cellulose and 5% polyethylene glycol 3350.

A subcoating solution was prepared as follows: 14.1 kg of water was added to a mixing vessel. 45 g of polyethylene glycol was added and mixed until all solids were dissolved. 855 g of hydroxyethyl cellulose was weighed and charged to the PEG solution while mixing. Mixing was continued until all solids were dissolved. The net weight of the prepared subcoating solution was determined by weighing.

9 g of compressed cores was charged to a coater and the cores were tumbled in the coater until a target exhaust temperature of 32° C. was achieved. The subcoating solution was applied to the cores while the coater was rotated at 12 rpm. Coating was continued until the target weight of 34 mg is achieved. At the end of the spray, the cores were removed from the coater.

The rate controlling membrane was completed as follows. The composition of the rate controlling membrane was as follows: 99% cellulose acetate and 1% poloxamer 188.

A membrane coating solution was prepared as follows: 47 kg of acetone was charged to a mixing vessel. The acetone was heated to 28° C. while the mixer was turned on. 25 g of poloxamer was added to the acetone and mixed until completely dissolved. 2.475 kg of cellulose acetate was added to the poloxamer solution, followed by addition of 475 g of purified water. The solution was mixed until all solids are in solution. The net weight of the prepared membrane coating solution was determined by weighing.

9 kg of subcoated cores were charged to a coater and the cores were tumbled in the coated until a target exhaust temperature of 32° C. was achieved. The membrane coating solution was applied to the cores while the coater was rotated at 12 rpm. Coating was continued until the target weight of 36 mg was achieved. At the end of spray, the cores were removed from the coater.

The exit orifice was drilled and the dosage forms were then dried as follows. A 1 mm orifice was drilled on the membrane coated cores using a laser drilling device. The drilled cores were then spread out on drying trays and dried at 40° C. at ambient humidity for up to 10 days.

### Example 26

### Topiramate Dosage Form

A drug core composition comprising 53.7 grams topiramate, 29.8 grams of CRODESTA F160, 10 grams of polyethylene oxide N-80 and 6 grams of polyethylene pyrrolidone K90, at less than 40 mesh particle sizes, were dry blended for about 30 minutes. The dry blend was then wetted with 20 grams of anhydrous ethyl alcohol SDA 3A while stirring to form a homogenous wet dough. The wet dough was passed thru #20 stainless steel screen to form noodles, and dried under a hood at ambient conditions for about 12 hours (overnight). The dried noodles were passed thru #20 stainless steel screen to form granules. These dried granules were then lubricated with 0.5 grams of <60 mesh magnesium stearate by roller blending for 3 minutes.

The push layer granulation was manufactured using the same process wherein 73.7 grams of polyethylene oxide 303, 20 grams of sodium chloride, 5 grams of polyvinyl pyrrolidone K2932, 1 gram of ferric oxide and 0.05 gram of BHT were dry blended for 30 minutes. The dry blend was then wetted with 80 grams of anhydrous ethyl alchol SDA 3A while stirring, to form a homogenous wet dough; The wet dough was then passed thru a #20 mesh stainless steel screen to form noodles. These noodles were dried for about 12 hours under a hood at ambient conditions. The dried noodles were then passed thru a #20 mesh stainless steel screen to form granules. These dried granules were then lubricated with 0.25 grams of stearic acid by roller blending for 3 minutes.

Both the drug and push layers were used to form a bilayer core using a 3/16-inch diameter LCT tooling. Drug layer granulation weighing 182 mg was introduced into the die first and then after slight tamping, the push layer granulation weighing 60 mg were then introduced and then compressed with a Carver Press at 0.75 ton compression force. This procedure was repeated until a desired amount of test tablets were produced. For initial trials 10 tablets were produced.

To these tablets, 3 layers of coating were applied. The first coating, a smoothing coating, provided a smooth surface for the succeeding rate-controlling membrane coatings. For the smoothing coating, 5 grams of poloxamer 407 were dissolved in 783 grams of de-ionized water by stirring. Then 45 grams of hydroxyethyl cellulose were introduced into the solution and stirred until a clear solution was achieved. An Aeromatic Coater was utilized for this coating. The 10 active tablets were mixed with placebo tablets (fillers) to provide a coater load of 500 grams. Standard Aeromatic Coating procedures were followed to coat about 3 to 4 mg of coating on each active tablet. The coated active tablets were dried in an oven at 40° C. and ambient humidity for about 12 hours.

The second coating was prepared by dissolving 77 grams of ethylcellulose (100 cps), 56 grams of hydroxypropyl cellulose EFX, and 7 grams of MYRJ 52S in 4,527 grams of warm ethanol SDA3A while stirring. Stirring was performed until a homogeneous solution was achieved. After stirring, the solution was sealed and stored at ambient conditions for about 2 days before application. An LDCS Vector Pan Coater was used for this coating. To achieve a 1.2 kg coater load, the 10 smooth coated active tablets were mixed with placebo filler tablets and coated with the second coating. Standard pan coating procedures were used for the coating process with a target coat of about 6 mils.

For the third coating, 87.5 grams of cellulose acetate 398-10 and 37.5 grams of LUTROL F68 were dissolved in 2,375 grams acetone with stirring and warming. This coating was applied using the same coater and standard coating procedure as with the second coat. After coating the active tablets were manually drilled to produce a 40 mil orifice, and then dried in an oven at 40° C. and ambient humidity for about 12 hours (overnight).

Drug release rates and residuals were determined as described in Example 11 from 5 of these tablets at intervals of 2 hours for 24 hours. The results show that topiramate was delivered at a substantially ascending rate of release for 12-14 hours. The time to deliver 90% of the 100 mg dose was about 16 hours. The cumulative delivery at 24 hours was 99%. The membranes were intact throughout the delivery pattern.

### Example 27

### Topiramate Dosage Form

Using the same granulation procedure described in Example 26, above, the following formulation consisting of 50 grams topiramate, 33.5 grams CRODESTA F-160, 10 grams polyethylene oxide N-80, and 6 grams of polyvinyl pyrrolidone K90, was wet granulated and lubricated with 0.5 gram and magnesium stearate. This constituted the drug layer with a load of 33.5% surfactant. Tablets were made following the procedures and materials described in Example 26.

Drug release rates were determined as described in Example 11. The results show that topiramate was delivered at a substantially ascending rate of release for 12-14 hours. The time to deliver 90% of the 100 mg dose was about 16 hours. The cumulative delivery at 24 hours was 99.5%. The membranes were intact throughout the delivery pattern.

### Example 28

### Topiramate Dosage Form

Tablets were made as described in Examples 26 and 27, but using a drug layer granulation consisting of 38.5% surfactant (CRODESTA F160). A push layer composition in the amount of 60 mg was used. Membrane compositions and amounts applied were approximately the same as counterpart tablets in Examples 26 and 27.

Drug release rates were determined on these tablets according to same procedures described in Example 11. The results show that topiramate was delivered at a substantially ascending rate of release for 14-16 hours. The time to deliver 90% of the 100 mg dose was about 17 hours. The cumulative delivery at 24 hours was 98.7%. The membranes were intact throughout the delivery pattern.

### Example 29

### Topiramate Dosage Form

Using standard procedures for fluid bed granulation, 288 grams of topiramate, 536 grams of CRODESTA F-160, 95.8 grams of polyethylene oxide N-80, and 5 grams of polyvinyl pyrrolidone were granulated. This granulation was then lubricated with 2 grams of stearic acid and 1 gram of magnesium stearate. A Glatt Fluid Bed Granulator (1 kg) capacity was utilized for this granulation.

To test if this granulation does or does not smear under manufacturing conditions, a tabletting run was performed with a multi-layer tablet press (Korsch Multi-Layer Tablet Press). Using the same tablet press and parameters, another tabletting run was performed using a counterpart granulation that contains poloxamer 407 as surfactant. It was observed that no smearing on the turret table and on the punches was observed with the granulation containing CRODESTA F160. In contrast; smearing was observed with the granulation containing poloxamer 407.

Therefore, the sugar ester surfactant provides an advantage in formulating dosage forms with respect to the poloxamer surfactant, and the sugar ester surfactant CRODESTA is another preferred surfactant for topiramate in the present invention.

### Examples 30-35

### Topiramate Dosage Forms

Tables 10-17 below list composition details for additional embodiments of the present invention. More particularly, the tables below provide details on the composition of tri-layer, controlled release, osmotic dosage forms containing topiramate. Said dosage forms comprised two drug compositions, wherein the amount and/or concentration of topiramate in the two drug compositions was different, and a push layer.

Each of the dosage forms described below was prepared according to the procedure described in Example 25, by selecting and substituting the suitable components.

Table 10 below lists the components of dosage forms as a function of total dosage of topiramate. For each layer or coating, weights are listed in milligrams (e.g for the drug layers, push layers, semi-permeable membranes, other coatings, etc.). Also listed in Table 1 are the sizes for each dosage form, and the orifice sizes on the dosage form, as prepared.

TABLE 10
Dosage Form Components

| Dosage | 10 mg | 20 mg | 40 mg | 80 mg | 120 mg | 160 mg |
|---|---|---|---|---|---|---|
| Size (inches) | 3/16 | 15/64 | 3/16 | 15/64 | 17/64 | 9/32 |
| Drug Layer 1 | 40 | 80 | 60 | 120 | 180 | 240 |
| Drug Layer 2 | 60 | 120 | 60 | 120 | 180 | 240 |
| Push Layer | 90 | 150 | 100 | 180 | 270 | 330 |
| Subcoat (Aqueous) | 15 | 20 | 15 | 20 | 25 | 25 |
| Membrane Coat (99/1 CA/Poloxamer) | 32 | 32 | 36 | 38 | 40 | 44 |
| Orifice Size (mil) | 1 × 40 | 1 × 40 | 1 × 40 | 1 × 40 | 2 × 60 | 2 × 80 |

CA = Cellulose Acetate

Table 11 below lists the components and amounts used in the preparation of the first drug composition for dosage forms comprising 40-160 mg total of topiramate. Target % (wt/wt) in granulation is the weight percent of the component as a function of the total weight of the drug layer.

TABLE 11
First Drug Composition (40-160 mg Dosages)

| | Target % (wt/wt) |
|---|---|
| Material ID | in Granulation |
| Topiramate | 29.67 |
| Polyethylene Oxide, NF, N-80, 200K, TG, LEO | 33.06 |
| Povidone, USP, Ph Eur, (K29-32) | 2.00 |
| Poloxamer 407, NF (Micronized) | 29.00 |
| METHYLCELLULOSE, USP, 15CPS, | 3.00 |
| (A15-LV-PREMIUM) | |
| Stearic Acid, NF, Ph Eur (Powder) | 3.00 |
| Magnesium Stearate, NF, Ph Eur | 0.25 |
| BHT, FCC, Ph Eur (Milled) | 0.02 |

Table 12 below lists the components and amounts used in the preparation of the second drug composition for dosage forms comprising 45-180 mg total of topiramate. Target % (wt/wt) in granulation is the weight percent of the component as a function of the total weight of the drug layer.

TABLE 12
Second Drug Composition (40-160 mg Dosages)

| | Target % (wt/wt) |
|---|---|
| Material ID | in Granulation |
| Topiramate | 37.00 |
| Povidone, USP, Ph Eur, (K29-32) | 2.00 |
| Poloxamer 407, NF (Micronized) | 54.65 |
| METHYLCELLULOSE, USP, 15CPS, | 3.00 |
| (A15-LV-PREMIUM) | |
| Ferric Oxide, NF, (Yellow) | 0.08 |
| Stearic Acid, NF, Ph Eur (Powder) | 3.00 |
| Magnesium Stearate, NF, Ph Eur | 0.25 |
| BHT, FCC, Ph Eur (Milled) | 0.02 |

Table 13 below lists the components and amounts used in the preparation of the first drug composition for dosage forms comprising 10-20 mg total of topiramate. Target % (wt/wt) in granulation is the weight percent of the component as a function of the total weight of the drug layer.

TABLE 13
First Drug Composition (10-20 mg dosages)

| | Target % (wt/wt) |
|---|---|
| Material ID | in Granulation |
| Topiramate | 6.25 |
| Polyethylene Oxide, NF, N-80, 200K, TG, LEO | 80.48 |
| Poloxamer 407, NF (Micronized) | 10.00 |
| Povidone, USP, Ph Eur, (K29-32) | 2.00 |
| Stearic Acid, NF, Ph Eur (Powder) | 1.00 |
| Magnesium Stearate, NF, Ph Eur | 0.25 |
| BHT, FCC, Ph Eur (Milled) | 0.02 |

Table 14 below lists the components and amounts used in the preparation of the second drug composition for dosage forms comprising 10-20 mg total of topiramate. Target % (wt/wt) in granulation is the weight percent of the component as a function of the total weight of the drug layer.

TABLE 14
Second Drug Composition (10-20 mg Dosages)

| | Target % (wt/wt) |
|---|---|
| Material ID | in Granulation |
| Topiramate | 12.50 |
| Polyethylene Oxide, NF, N-80, 200K, TG, LEO | 69.22 |
| Poloxamer 407, NF (Micronized) | 15.00 |
| Povidone, USP, Ph Eur, (K29-32) | 2.00 |
| Iron Oxide, Red | 0.01 |
| Stearic Acid, NF, Ph Eur (Powder) | 1.00 |
| Magnesium Stearate, NF, Ph Eur | 0.25 |
| BHT, FCC, Ph Eur (Milled) | 0.02 |

Table 15 below lists the components and amounts used in the preparation of the push layer for all dosage forms of topiramate. Target % (wt/wt) in granulation is the weight percent of the component as a function of the total weight of the drug layer.

TABLE 15
Push Layer Composition

| | Target % (wt/wt) |
|---|---|
| Material ID | in Granulation |
| Polyethylene Oxide, NF, 303, 7000K, TG, LEO | 74.30 |
| Sodium Chloride, USP, Ph Eur, (Powder) | 20.00 |
| Povidone, USP, Ph Eur, (K29-32) | 5.00 |
| Ferric Oxide, NF, (Red) | 0.10 |
| Ferric Oxide, NF, (Yellow) | 0.30 |
| Stearic Acid, NF, Ph Eur, (Powder) | 0.25 |
| BHT, FCC, Ph Eur, (Milled) | 0.05 |

Table 16 below lists the components and amounts used in the preparation of the subcoat (aqueous subcoat) for all dosage forms of topiramate. Target % (wt/wt) in subcoat formulation is the weight percent of the component as a function of the total weight of the subcoat.

TABLE 16
Subcoat Composition

| | Target % (wt/wt) in |
|---|---|
| Material ID | Subcoat Formulation |
| Hydroxyethyl Cellulose, NF | 95 |
| Polyethylene Glycol 3350, NF, Ph Eur, LEO | 5 |

Tables 17 below lists the components and amounts used in the preparation of the CA (cellulose acetate) membrane coat, for all dosage forms of topiramate. Target % (wt/wt) in subcoat formulation is the weight percent of the component as a function of the total weight of the subcoat.

TABLE 17
CA Membrane Coat

| | Target % (wt/wt) in |
|---|---|
| Material ID | Subcoat Formulation |
| Cellulose Acetate, NF, (398-10) | 99 |
| Poloxamer 188, NF, Ph Eur | 1 |

In as much as the foregoing specification comprises disclosed embodiments, it is understood what variations and modifications may be made herein, in accordance with the principles disclosed, without departing from the invention.

1. A drug formulation comprising granules having a substrate and a coating, said granule substrate comprising a solubilizing agent or a low solubility therapeutic drug, or both, and said granule coating comprising a hydrophilic polymer.

2. A drug formulation as in claim 1, wherein the granule substrate comprises a solubilizing agent.

3. A drug formulation as in claim 2, wherein the solubilzing agent is a surfactant.

4. A drug formulation as in claim 3, wherein the granule substrate comprises a low solubility therapeutic drug.

5. A drug formulation as in claim 4, wherein the low solubility therapeutic drug is topiramate.

6. A drug formulation as in claim 4, wherein the amount of surfactant is between about 5% and about 50% by weight of the core.

7. A drug formulation as in claim 4, wherein the surfactant is selected from the group consisting of polyoxyl 40 stearate, polyoxyl 50 stearate, triblock co-polymers of ethylene oxide/propylene oxide/ethylene oxide, sorbitan monopalmitate, sorbitan monostearate, glycerol monostearate, polyoxyethlene stearate, polyoxyethylene 40 sorbitol lanolin derivative, polyoxyethylene 75 sorbitol lanolin derivative, polyoxyethylene 6 sorbitol beeswax derivative, polyoxyethylene 20 sorbitol beeswax derivative, polyoxyethylene 20 sorbitol lanolin derivative, polyoxyethylene 50 sorbitol lanolin derivative, polyoxyethylene 23 lauryl ether, polyoxyethylene 23 lauryl ether, polyoxyethylene 2 cetyl ether, polyoxyethylene 10 cetyl ether, polyoxyethylene 20 cetyl ether, polyoxyethylene 2 stearyl ether, polyoxyethylene 10 stearyl ether, polyoxyethylene 20 stearyl ether, polyoxyethylene 21 stearyl ether, polyoxyethylene 20 oleyl ether, polyoxyethylene 40 stearate, polyoxyethylene 50 stearate, polyoxyethylene 100 stearate, sorbitan monopalmitate, sorbitan monostearate, sorbitan tristearate, polyoxyethylene 4 sorbitan monostearate, polyoxyethylene 20 sorbitan tristearate and mixtures thereof.

8. A drug formulation as in claim 7, wherein the surfactant is selected from the group consisting of polyoxyl 40 stearate, polyoxyl 50 stearate, di-block copolymers of ethylene oxide:propylene oxide, and a:b:a triblock copolymers of ethylene oxide:propylene oxide:ethylene oxide.

9. A drug formulation as in claim 4, wherein the low solubility therapeutic drug in the absence of solubilizing agent has an aqueous solubility between about 1 μg/ml and about 50 mg/ml.

10. A drug formulation as in claim 1, wherein the granule coating is continuous.

11. A drug formulation as in claim 1, wherein the granule coating is water-soluble.

12. A drug formulation as in claim 11, wherein the hydrophillic polymer is selected from the group consisting of polyvinyl pyrrolidone, polyethylene oxide, polyethylene glycol, polyvinyl alcohol, polyvinyl alcohol-polyethylene glycol copolymer, vinyl acetate-vinyl pyrrolidone copolymer, hydroxypropyl cellulose, hydroxypropyl methylcellulose, methylcellulose, hydroxyethyl cellulose, sodium carboxymethylcellulose, calcium carboxymethylcellulose, polyvinyl acetate polyethylene glycol co-polymer, a starch, maltodextrin, a sugar, sorbitol, mannitol, sucrose, gelatin, casein, a natural gum, an alginate or mxtures thereof.

13. A drug formulation as in claim 1, wherein the granule coating is water insoluble.

14. A drug formulation as in claim 13, wherein the hydrophillic polymer is selected from the group consisting of a polyether, a polyester, cellulose acetate, cellulose acetate butyrate, a polyacrylate and mixtures thereof.

15. A drug formulation as in claim 1, wherein the granule coating consists of a single layer.

16. A drug formulation as in claim 15, wherein the single layer granule coating comprises a hydrophillic polymer selected from polyvinyl pyrrolidone, polyethylene oxide or methylcellulose.

17. A drug formulation as in claim 16, wherein the hydrophillic polymer is methylcellulose.

18. A drug formulation as in claim 1, wherein the granule coating comprises a multiplicity of layers.

19. A drug formulation as in claim 18, wherein the granule coating consists of two layers.

20. A drug formulation as in claim 19, wherein the granule coating comprises an outer coating layer of methylcellulose and an inner coating layer of polyethylene oxide or polyvinyl pyrrolidone.

21. A dosage form comprising a drug formulation as in claim 1, wherein the granule comprises a low solubility therapeutic drug and wherein the low solubility therapeutic drug is topiramate.

22. A dosage form comprising
(a) a core comprising a first drug composition, a second drug composition and a push layer comprising an osmopolymer; wherein each of the first and second drug compositions comprise granules having a substrate and a coating, said granule substrate comprising a solubilizing agent and a low solubility therapeutic drug, and said granule coating comprising a hydrophilic polymer.
(b) a semi-permeable wall surrounding the core; and
(c) an exit orifice through the semi-permeable wall for releasing the drug compositions from the dosage form over a prolonged period of time.

23. A dosage form as in claim 22, wherein the low solubility therapeutic drug is topiramate.

24. A dosage form as in claim 23, wherein the solubilizing agent is a surfactant.

25. A dosage form as in claim 24, wherein the hydrophilic polymer is methylcellulose.

26. A dosage form comprising
(a) a core comprising a drug composition, wherein the drug composition comprise granules having a substrate and a coating, said granule substrate comprising a low solubility therapeutic drug and optionally a solubilizing agent, and said granule coating comprising one or more layers, wherein each granule coating layer comprises an independently selected hydrophilic polymer;
(b) a semi-permeable wall surrounding the core; and
(c) an exit orifice through the semi-permeable wall for releasing the drug compositions from the dosage form over a prolonged period of time.

27. A dosage form as in claim 26, wherein the number of granule coating layers is selected to (a) decrease the dissolution rate of the therapeutic drug or (b) increase the time to maximum release rate of the therapeutic drug or (c) increase the period of therapeutic drug delivery or (d) decrease the magnitude of the maximum release rate of the therapeutic drug.

28. A dosage form as in claim 26, wherein the granule coating comprises one layer and wherein the hydrophilic polymer is selected to (a) decrease the dissolution rate of the therapeutic drug or (b) increase the time to maximum release rate of the therapeutic drug or (c) increase the period of therapeutic drug delivery or (d) decrease the magnitude of the maximum release rate of the therapeutic drug.

29. A dosage form as in claim 26, wherein the granule coating comprises one layer and wherein the amount of said coating is selected to (a) decrease the dissolution rate of the therapeutic drug or (b) increase the time to maximum release rate of the therapeutic drug or (c) increase the period of therapeutic drug delivery or (d) decrease the magnitude of the maximum release rate of the therapeutic drug.

30. A dosage form as in claim 26, wherein the dissolution rate of the therapeutic drug or the time to maximum release rate of the therapeutic drug or the period of therapeutic drug delivery or the magnitude of the maximum release rate of the therapeutic drug is controlled by varying one or more of (a) the number of coating granule layers, (b) the amount of each coating granule layer or (c) the hydrophilic polymer of each coating granule layer.

31. A dosage form as in claim 26, wherein the low solubility therapeutic drug is topiramate, wherein the granule coating comprises one layer and wherein the hydrophillic polymer is selected from the group consisting of methylcellulose, polyvinylpyrolidone and polyethylene oxide.

32. A dosage form as in claim 31, wherein the amount of granule coating is in the range of between about 2.5% and about 23% by weight.

33. A dosage form as in claim 26, wherein the granule coating comprises one layer and the hydrophilic polymer is selected from the group consisting of (a) methylcellulose in an amount in the range of between about 2.5% and about 13% by weight, (b) polyvinylpyrolidone in an amount in the range of between about 3% and about 23% by weight and (c) polyethylene oxide in an amount in the range of about 5% to about 15% by weight.

34. A dosage form as in claim 26, wherein the granule coating comprises one layer and the hydrophilic polymer is methylcellulose in an amount of about 3% by weight.
